(19) United States
(12) Patent Application Publication
(10) Pub. No.: US 2007/0219216 A1
(43) Pub. Date: Sep. 20, 2007

(54) 1-Alkylpiperazinyl-Pyrrolidin-2,5-Dione Derivatives as Adrenergic Receptor Antagonists

(76) Inventors: Nitya Anand, Uttar Pradesh (IN); Mohammad Salman, Princeton, NJ (US); Somesh Sharma, New Delhi, IN (US); Gobind Singh Kapkoti, Haryana (IN); Praful Gupta, Saharaapur (IN); Anurag Mishra, New Delhi, IN (US)

Correspondence Address: RANBAXY INC., 600 COLLEGE ROAD EAST, SUITE 2100, PRINCETON, NJ, 08540, US

(21) Appl. No.: 10/575,606

(22) PCT Filed: Oct. 14, 2004
(86) PCT No.: PCT/IB04/03362

§ 371 Date: Feb. 28, 2007

(30) Foreign Application Priority Data
Oct. 15, 2003 (IB) PCT/IB03/04545

(51) Int. Cl. A61K 31/496; A61P 13/08; C07D 207/408; C07D 209/48

(52) U.S. Cl. 514/254.01; 544/372

(57) ABSTRACT

This invention relates to α₁ₐ and/or α_{1d} adrenergic receptor antagonists of formula 1 Compounds disclosed herein can function as α₁ₐ and/or α_{1d} adrenergic receptor antagonists and can be used for the treatment of diseases or disorders mediated through α₁ₐ and/or α_{1d} adrenergic receptors. Compounds disclosed herein can be used for the treatment of benign prostatic hyperplasia and related symptoms thereof. Compounds disclosed herein can also be used for the treatment of lower urinary tract symptoms associated with or without benign prostatic hyperplasia. The invention also relates to a process for the preparation of compounds disclosed herein, pharmaceutical compositions containing these compounds and the methods of treating diseases or disorders mediated through α₁ₐ and/or α_{1d} receptors.

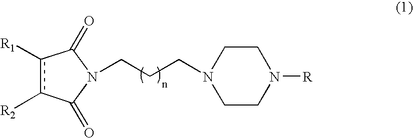

### FIELD OF THE INVENTION

This invention relates to α₁ₐ and/or α_{1d} adrenergic receptor antagonists. Compounds disclosed herein can function as α₁ₐ and/or α_{1d} adrenergic receptor antagonists and can be used for the treatment of diseases or disorders mediated through α₁ₐ and/or α_{1d} adrenergic receptors. Compounds disclosed herein can be used for the treatment of benign prostatic hyperplasia and related symptoms thereof. Compounds disclosed herein can also be used for the treatment of lower urinary tract symptoms associated with or without benign prostatic hyperplasia. The invention also relates to a process for the preparation of compounds disclosed herein, pharmaceutical compositions containing these compounds and the methods of treating diseases or disorders mediated through α₁ₐ and/or α_{1d} receptors.

### BACKGROUND OF THE INVENTION

Benign prostatic hyperplasia (BPH) is a condition which develops in elderly males and refers to the benign overgrowth of the stromal and epithelial elements of the prostate associated with aging. The symptoms of BPH vary, but the most common ones involve changes or problems with urination, such as a hesitant, interrupted, weak stream or urgency and leaking or dribbling or more frequent urination, especially at night. Consequences of BPH can involve hypertrophy of bladder smooth muscle, a decompensated bladder and an increased incidence of urinary tract infection.

There are two components of BPH, static and a dynamic component. The static component is due to enlargement of the prostate gland, which may result in compression of the urethra and obstruction to the flow of urine from the bladder. The dynamic component is due to increased smooth muscle tone of the bladder neck and prostate itself and is regulated by α-1 adrenergic receptor.

Currently, the most effective treatment for BPH is the surgical procedure of transurethral resection of the prostate (TURP), since it removes the obstructing tissue (C. Chapple's Br. Med. Journal 304: 1198-1199, 1992). It is a treatment, which is directed to the static and dynamic components of the BPH. However this surgical treatment is associated with rates of mortality (1%) and adverse event (incontinence 2-4%) infection 5-10%, and impotence 5-10%. A non invasive alternative treatment is therefore highly desirable. There are some drug therapies, which address the static component of this condition. Administration of finasteride is one such therapy, which is indicated for the treatment of symptomatic BPH. This drug is a competitive inhibitor of the enzyme 5α-reductase which is responsible for the conversion of testosterone to dihydrotestosterone in the prostate gland. Dihydrotestosterone appears to be the major mitogen for prostate growth, and agents which inhibit 5α reductase reduce the size of the prostate and improve urine flow through the prostatic urethra. Although finasteride is a potent 5α reductase inhibitor and causes a marked decrease in serum and tissue concentrations of dihydrotestosterone, it is only moderately effective in the treatment of symptomatic BPH. The effects of finasteride take 6-12 months to become evident, and for many men the clinical development is minimal.

The dynamic component of BPH has been addressed by the use of adrenergic receptor blocking agents, which act by decreasing the smooth muscle tone within the prostate gland. A variety of α₁ adrenergic receptor antagonists such as terazosin, doxazosin, prazosin, alfuzosin and tamulosin have been investigated for the treatment of symptomatic bladder outlet obstruction due to BPH. However, these drugs are associated with vascular side effects (e.g. postural hypertention, syncope, dizziness, headache etc) due to lack of selectivity of action between prostatic and vascular α₁-adrenoceptors. There are several lines of evidence to suggest that selectivity for α₁ₐ adrenoceptor over α_{1b} adrenoceptor will result in relative lack of vascular side effects, thus lead to a better tolerability. In-vivo studies in healthy subjects comparison of α₁ₐ/α_{1d} selective antagonists (e.g., tamsulosin) or α₁ₐ selective antagonists (e.g., urapidil) with non selective antagonists (e.g., doxazosin, prazosin, or terazosin) under a variety of experimental conditions (e.g., involving the administration of exogenous agonist or release of endogenous agonist by cold stimulation) in several vascular beds including the skin circulation in finger tips, the dorsal hand vein, or with total peripheral resistance have been reported. (Eur. J. Clin. Pharmacol, 1996, 49,371-375; Naunyn Schmiedeberg's Arch. Pharmacol. 1996, 354, 557-561; Jpn. J. Pharmacol. 1999, 80, 209-215; Br J: Clin. Pharmacol. 1999, 47, 67-74). These studies have reported that an antagonist with high affinity for α₁ₐ or α₁ₐ/α_{1d} can cause some degree of vasodilation but that it is much smaller than with non-subtype-selective α₁ adrenoceptor antagonist. Further, there is increased vascular alb adrenoceptor expression in elderly patients and thus α₁ₐ/α_{1d} selective agents with selectivity over α_{1b} adrenoceptor subtype would be of particular importance in benign prostatic hyperplasia, which is generally a disease of old age. Antagonism of both α₁ₐ adrenoceptor and α_{1d} adrenoceptor is believed important to relieve lower urinary tract symptoms especially associated (suggestive of) with BPH. Targeting α₁ₐ adrenoceptor with antagonists is important in relaxing prostate smooth muscle and relieving bladder outlet obstruction whereas α_{1d} adrenoceptor antagonism is important to target irritative symptoms.

Over the past decade, there has been an intensive search for selective α₁ adrenoceptor antagonists for benign prostatic hyperplasia which would avoid the cardiovascular side effects associated with currently used drugs. Selective antagonists have been described by Hieble et al. in Exp. Opin. Invest. Drugs; 6, 367-387 (1997) and by Kenny et al., in J. Med. Chem.; 40, 1293-1325 (1995). Pharmacological activities associated with phenyl piperazines have been studied in, Eur. J. Med. Chem.—Chimica Therapeutica, 1, 173-176 (1977), which describes substituted trifluoromethyl phenyl piperazines having cyclo-imido alkyl side chains shown below.

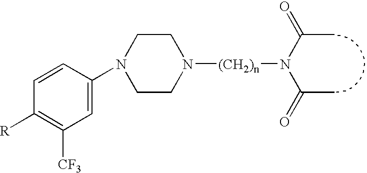

These compounds are potential anorectic agents with no CNS side effects. Other compounds which have been prepared as anxiolytic, neuroleptic, anti-diabetic and anti-allergic agents are described in the following references: Yukihiro et al.; PCT Appl. WO 98/37893 (1998), Steen et al.; J. Med. Chem., 38, 4303-4308 (1995), Ishizumi et al. Chem. Pharm. Bull; 39 (9), 2288-2300 (1991), Kitaro et al.; JP 02-235865 (1990), Ishizumi et al.; U.S. Pat. No. 4,598,078 (1986), New et. al; J. Med. Chem. 29, 1476-1482 (1986), Shigeru et. al; JP 60-204784 (1985), New et al., U.S. Pat. No. 4,524,206 (1985), Korgaonkar et al.; J. Indian Chem. Soc., 60, 874-876 (1983).

The synthesis and pharmacology of some 2-[3-(4-aryl-1-piperazinyl)propyl]-1H-benz(de) isoquinolin-1,3-(2H)-diones/2,5-pyrrolidinediones (J. Indian. Chem. Soc. Vol., LXIII, 529-530 (1986), of N—(N⁴-aryl-N¹-piperozinylmethyl)-4-(4-methoxyphenyl)piperidine-2,6-diones [J. Indian Chem. Soc., Vol. LV, 819-821 (1978)], and of N—(N⁴-arylpiperazinylalkyl)-phthalmides (J. Indian. Chem. Soc., Vol. LVI, 1002-1005 (1979)] have been reported. The compounds were shown to exhibit antihypertensive and CNS depressant activity in experimental animals. However, none of the above mentioned references disclose or suggest the α₁ subtype selectivity profile of the compounds disclosed therein and thus their usefulness in the treatment of symptoms of benign prostate hyperplasia did not arise.

The synthesis of 1-(4-arylpiperazin-1-yl)-ω-[N-(α,ω-dicarboximido)]-alkanes useful as uro-selective α₁-adrenoceptor blockers are disclosed in U.S. Pat. Nos. 6,083,950, 6,090,809, 6,410,735, 6,420,559 and 6,420,366. These compounds have good α₁-adrenergic blocking activity and selectivity.

Other reports describing selective α₁ adrenoceptor antagonists are U.S. Pat. Nos. 6,376,503, 6,319,932, and 6,339,090, EP 711757, WO 02/44151; 99/42448, 99/42445, 98/57940, 98/57632, 98/30560 and WO 97/23462, and all these patents are incorporated by reference herein in their entirety.

### SUMMARY OF THE INVENTION

Provided herein are α₁ₐ and/or α_{1d} adrenergic receptor antagonists which are useful as safe and effective treatment of benign prostatic hyperplasia or related symptoms thereof, and method for the syntheses of these compounds.

Also provided herein are pharmaceutical compositions containing the compounds, which may also contain pharmaceutically acceptable carriers, excipients or diluents which are useful for the treatment of benign prostatic hyperplasia or related symptoms thereof.

Also provided herein are the enantiomers, diastereomers, pharmaceutically acceptable salts pharmaceutically acceptable, solvates, polymorphs, N-oxides or metabolites of these compounds having the same type of activity.

Pharmaceutical compositions comprising the compounds of the invention, their enantiomers, diastereomers, polymorphs, pharmaceutically acceptable salts, pharmaceutically acceptable solvates, N-oxides or metabolites, in combination with pharmaceutically acceptable carriers and optionally included excipients are also provided herein.

Other aspects will be set forth in the description which follows, and in part will be apparent from the description or may be learnt by the practice of the invention.

In accordance with one aspect of the present invention, there is provided a compound having the structure of Formula I,

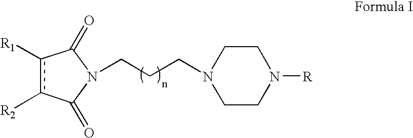

its pharmaceutically acceptable acid addition salts, solvates, enantiomers, diastereomers, regioisomers, N-oxides, polymorphs, prodrugs and metabolites wherein,

— represents no bond or a single bond;

The variable n can represent an integer 1 to 2.

R₁ and R₂ can represent alkyl, cycloalkyl, or 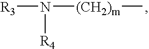
wherein m can represent an integer 0 or 1.
R₃ can represent alky, or cycloalkyl.
R₄ can represent hydrogen or alkyl.
R₂ can also represent hydrogen.
R₁ and R₂ together can represent cycloalkyl or cycloalkenyl.
R can represent 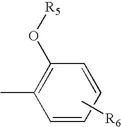
wherein, R₅ can represent alkyl or cycloalkyl.
R₆ can represent hydrogen, halogen or alkyl.

In accordance with a second aspect, there is provided a method for the treatment of a patient suffering from a disease or disorder mediated through α₁ₐ and/or α_{1d} adrenergic receptor, comprising administering to a patient in need thereof, an effective amount of adrenergic receptor antagonist.

In accordance with a third aspect, there is provided a method for the treatment of a patient suffering from benign prostatic hyperplasia and related symptoms, comprising administering to a patient in need thereof, an effective amount of adrenergic receptor antagonist compounds as described above.

In accordance with a fourth aspect, there is provided a method for the treatment of a patient suffering from lower urinary tract symptoms, for example, irritative symptoms such as frequent urination, urgent urination, nocturia and unstable bladder contractions, obstructive symptoms such as hesitancy, poor stream, prolong urination, and feelings of incomplete emptying, comprising administering to a patient in need thereof, an effective amount of adrenergic receptor antagonist compounds as described above.

In accordance with a fifth aspect, there are provided processes for preparing the compounds as described above.

In accordance with a sixth aspect, there is provided a method for the treatment of a patient suffering from benign prostatic hyperplasia and related symptoms, comprising administering to a patient in need thereof, an effective amount of a compound (or composition) described above in combination with a selective muscarinic receptor antagonist.

In accordance with a seventh aspect, there is provided a method for the treatment of a patient suffering from benign prostatic hyperplasia and related symptoms, comprising administering to a patient in need thereof, an effective amount of a compound (or composition) described above in combination with and a testosterone 5α-reductase inhibitor.

In accordance with an eight aspect, there is provided a method for the treatment of a patient suffering from benign prostatic hyperplasia and related symptoms, comprising administering to a patient in need thereof, an effective amount of a compound (or composition) described above in combination with a selective muscarinic receptor antagonist and optionally included a testosterone 5α-reductase inhibitor.

Receptor binding and in vitro functional assay studies described below indicated that the compounds disclosed herein possess selective and potent α₁ₐ adrenoceptor antagonistic activity over the α_{1b} and/or α_{1d} adrenoceptors. The examples presented below describe a method to treat BPH in a patient wherein the test compounds alleviated pressure at dosages, which did not result, in significant change in blood pressure. Several of the compounds disclosed herein demonstrated manifest selectivity for prostatic tissues in comparison to known compounds. Additionally, the compounds disclosed herein are also useful for relaxing lower urinary tract tissues and thus alleviating irritative symptoms in patient. Therefore, the pharmaceutical compositions are useful for the treatment of diseases or disorders mediated through α₁ₐ adrenoceptor. Compounds disclosed herein can also be used for the treatment of lower urinary tract symptoms. Compounds and compositions described herein can be administered orally, parenterally or topically.

The following definitions apply to the terms as used herein:

The term “alkyl” refers to straight or branched, saturated hydrocarbon having one to three carbon atom(s). One or more hydrogen atom(s) of said alkyl can optionally be replaced by halogen, cycloalkyl, alkynyl. Examples of alkyl, but are not limited to, include methyl, isopropyl, 1,1,1 trifluoroethane and the like.

The term “cycloalkyl” refers to saturated carbocyclic ring having three to seven carbon atoms. Example of cycloalkyl, but are not limited to, include cyclopropyl, cycloburyl and cyclopentyl, and the like.

The term “cycloalkenyl refers to unsaturated carbocyclic ring having three to seven carbon atoms. Example of cycloakenyl, but are not limited to, include cyclopropenyl and cyclobutenyl, and the like.

The said “cycloalkyl” or cycloalkenyl” may optionally be substituted with halogen.

### DETAILED DESCRIPTION OF THE INVENTION

The compounds described herein may be prepared by techniques well known in the art and familiar to the average synthetic organic chemist. In addition, the compounds of the present invention may be prepared by the following reaction sequences as depicted in Schemes I, II, III, IV and V.

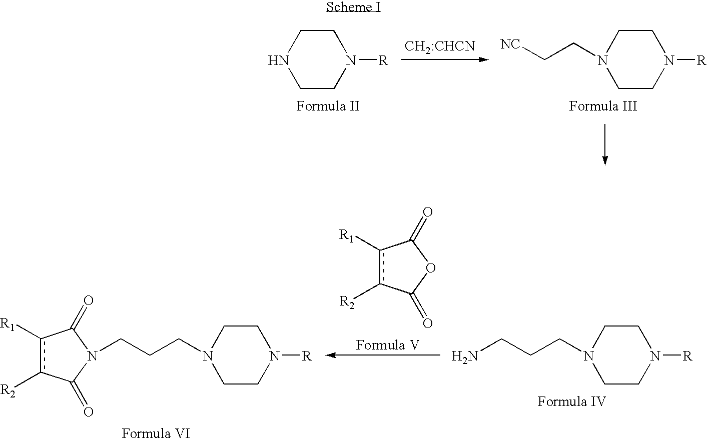

The compound of Formula VIII can be prepared according to Scheme I. Thus, reacting a compound of Formula II with acrylonitrile to give a compound of Formula III (wherein R is the same as defined earlier), which on hydrogenation gives a compound of Formula IV, which on treatment with a compound of Formula V gives a compound of Formula VIII (wherein R₁ and R₂ are the same as defined earlier), which can then be further, converted to any pharmaceutically acceptable salt known to one ordinary skilled in art.

The reaction of a compound of Formula II with acrylonitrile to give a compound of Formula III can be carried out in a solvent, for example, chloroform, methanol, ethanol, cyclohexane, acetonitrile, n-butylalcohol, dichloromethane, dimethylsulfoxide, tetrahydrofuran or dimethylformamide.

The reaction of a compound of Formula II with acrylonitrile can be carried out in the presence of an organic base, for example, diethylamine, triethylamine, tributylamine, pyridine, 4-dimethylaminopyridine or ethyl diisopropylamine.

The hydrogenation of a compound of Formula III to give a compound of Formula IV can be carried out in presence of Raney-Nickel/hydrogen and ammonia or Palladium-carbon/hydrogen in an alcoholic solvent, for example, methanol, ethanol or isopropyl alcohol.

The reaction of a compound of Formula IV with a compound of Formula V to give a compound of Formula VI can be carried out in a solvent, for example, acetonitrile, toluene, xylene, tetrahydrofuran, benzene, dichloromethane, acetic anhydride or chloroform.

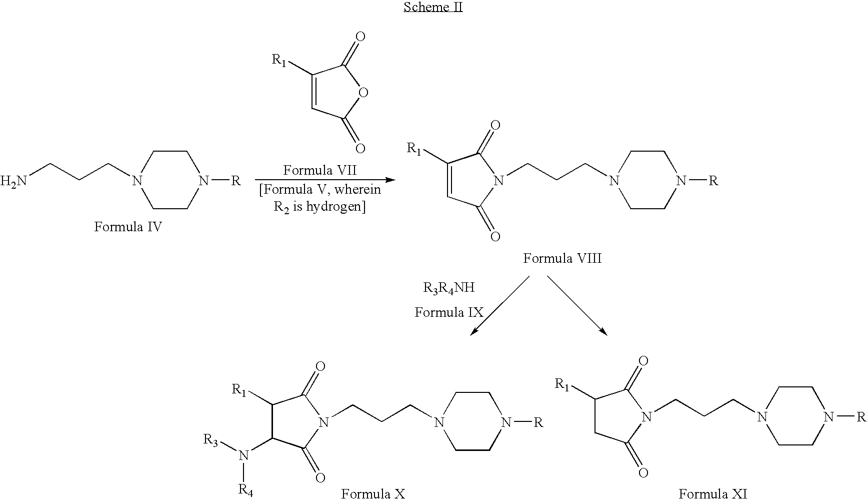

The compounds of Formula X and XI can be prepared according to Scheme II. Thus, reacting a compound of Formula IV with a compound of Formula VIII gives a compound of Formula VIII (wherein R and R₁ are the same as defined earlier), which,

a) on treatment with a compound of Formula IX gives a compound of Formula X (wherein R₃ and R₄ are the same as defined earlier).

b) on reduction gives a compound of Formula XI.

The compounds of Formula X and XI can then be further converted to any pharmaceutically acceptable salt known to one ordinary skilled in art.

The reaction of a compound of Formula IV with a compound of Formula VII to give a compound of Formula VIII can be carried out in a solvent, for example, acetonitrile, toluene, xylene, benzene, dichloromethane, tetrahydrofuran, acetic anhydride or chloroform.

The reaction of a compound of Formula VIII with a compound of Formula IX to give a compound of Formula X can be carried out in a solvent, for example, methanol, ethanol, tetrahydrofuran, chloroform, acetonitrile, dimethylsulfoxide, dimethylformamide, cyclohexane, dichloromethane, methanol and tetrahydrofuran, methanol and acetonitrile or methanol and cyclohexane.

The reduction of compound of Formula VIII to give a compound of Formula XI can be carried out in presence of a reducing agent, for example, Palladium-Carbon/hydrogen, or Raney Nickel/hydrogen and ammonia in an alcoholic solvent, for example, ethanol, methanol or isopropyl alcohol.

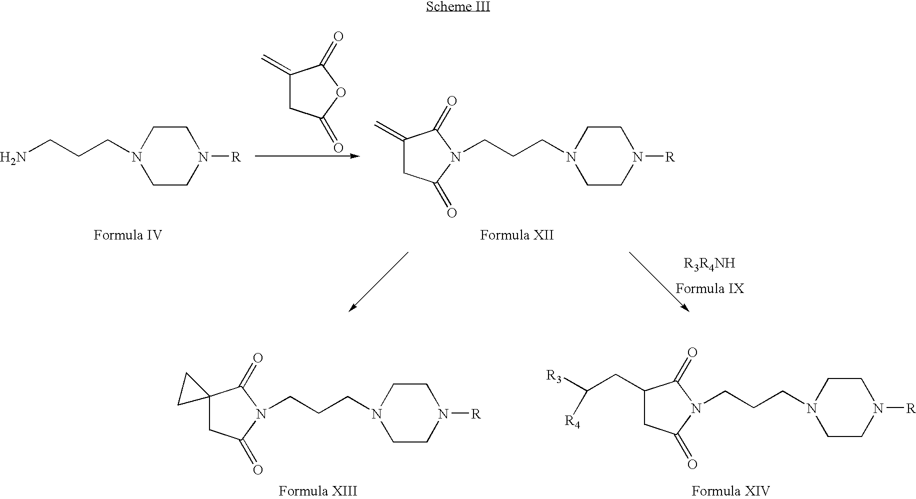

The compounds of the Formula XIII and XIV can be prepared according to the Scheme III. Thus, reacting a compound of Formula IV with itaconic anhydride to give a compound of Formula XII (wherein R is the same as defined earlier), which on treatment

a) with a methylene transfer agent, for example, trimethylsulphoxonium iodide or diazomethane gives a compound of Formula XIII.

b) with a compound of Formula IX gives a compound of Formula XIV (wherein R₃ and R⁴ are same as defined earlier).

The compounds of Formula XIII and XIV can then be converted to any pharmaceutically acceptable salt known to one ordinary skilled in art.

The reaction of compound of Formula IV with itaconic anhydride to give a compound of Formula XII can be carried out in a solvent, for example, acetonitrile, toluene, xylene, benzene, dichloromethane, tetrahydrofuran, acetic anhydride or chloroform.

The reaction of a compound of Formula XII with a methylene transfer agent, for example, trimethylsulphoxonium iodide or diazomethane to give a compound of Formula XIII can be carried out in a solvent, for example, dimethylsulfoxide, dimethylformamide, acetonitrile, tetrahydrofuran, ethanol or methanol.

The reaction of compound of Formula XII with compound of Formula IX to give a compound of Formula XIV can be carried out in a solvent, for example, methanol, ethanol, tetrahydrofuran, chloroform, acetonitrile, dimethylsulfoxide, dimethylformamide, cyclohexane, dichloromethane, methanol and tetrahydrofuran, methanol and acetonitrile or methanol and cyclohexane.

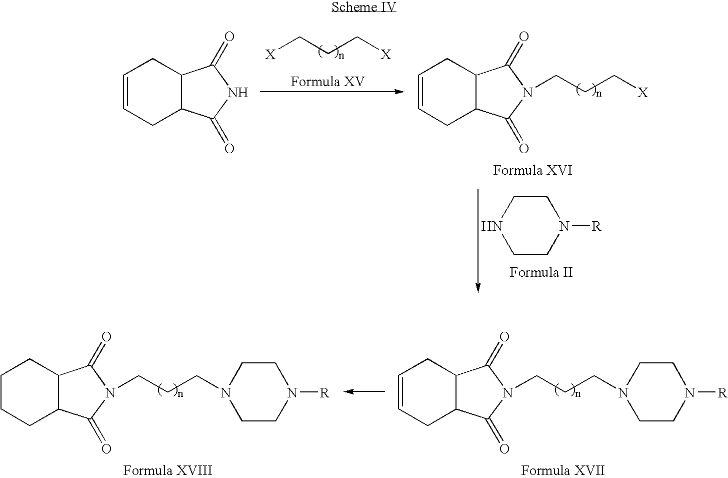

The compounds of Formula XVIII can be prepared according to Scheme IV. Thus reacting 3α,4,7,7α-tetrahydro-isoindole-1,3-dione with a compound of Formula XV to give a compound of Formula XVI (wherein X is a halogen and n is the same as defined earlier) which on further treatment with a compound of Formula II gives a compound of Formula XVII, which on hydrogenation gives a compound of Formula XVIII, which can then be further, converted to any pharmaceutically acceptable salt known to one ordinary skilled in art.

The reaction of 3α,4,7,7α-tetrahydro-isoindole-1,3-dione with a compound of Formula XV to give a compound of Formula XVI can be carried out in a solvent, for example, acetone, methyl ethylketone, diisopropyl ketone, tetrahydrofuran, dimethylformamide or dimethylsulfoxide.

The reaction of 3α,4,7,7α-tetrahydro-isoindole-1,3-dione with a compound of Formula XV to give a compound of Formula XVI can be carried out in presence of an inorganic base, for example, potassium carbonate, barium carbonate, cesium carbonate, calcium carbonate, sodium carbonate, potassium bicarbonate or sodium bicarbonate and an organic or inorganic halide, for example, tetra-n-butylammonium chloride, tetra-n-butylammonium bromide or potassium iodide.

The reaction of a compound of Formula XVI with a compound of Formula II to give a compound of Formula XVII can be carried out in a solvent, for example, dimethylformamide, dimethyl sulfoxide, acetonitrile, ethanol, methanol, isopropyl alcohol, tetrahydrofuran or chloroform.

The reaction of a compound of Formula XVI with a compound of Formula II to give a compound of Formula XVII can be carried out in presence of a base, for example, potassium carbonate, potassium bicarbonate, sodium carbonate, sodium bicarbonate, triethylamine, ammonium hydroxide, pyridine or 4-dimethylaminopyridine.

The hydrogenation of a compound of Formula XVII to give a compound of Formula XVIII can be carried out in presence of Palladium-Carbon/hydrogen or Raney Nickel in an alcoholic solvent, for example, ethanol, methanol or isopropyl alcohol.

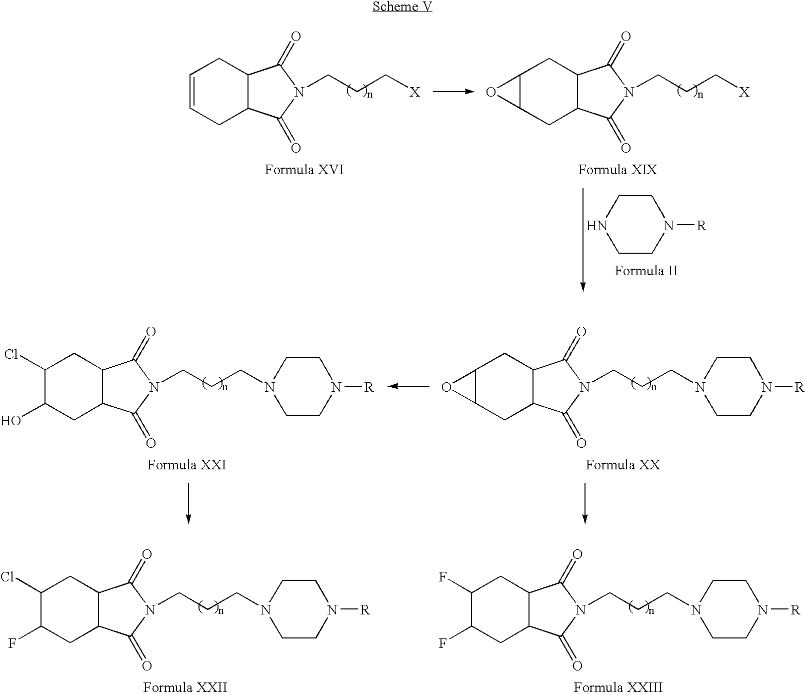

The compounds of Formula XXII and XXIII can be prepared according to Scheme V. Thus reacting a compound of Formula XVI with a peroxyacid, for example, m-chloroperbenzoic acid to give a compound of Formula XIX (wherein X is a halogen and n is the same as defined earlier), which on treatment with a compound of Formula II gives a compound of Formula XX (wherein R is as defined earlier), which on

(a) treatment with hydrochloric acid gives a compound of Formula XXI, which on reaction with a fluorinating agent gives a compound of Formula XXII.

(b) which on reaction with a fluorinating agent gives a compound of Formula XXIII.

The compounds of Formula XXII and XXIII can further be converted to any pharmaceutically acceptable salt known to one ordinary skilled in art.

The reaction of compound of Formula XVI with a peroxyacid, for example, m-chloroperbenzoic acid to give a compound of Formula XIX can be carried out in a solvent, for example, chloroform, methanol, acetone, dichloromethane, acetonitrile or tetrahydrofuran.

The reaction of compound of Formula XIX with a compound of Formula II to give a compound of Formula XX can be carried out in a solvent, for example, acetonitrile, ethanol, butanol, halogenated solvents, tetrahydrofuran, dimethylformamide or dimethylsulfoxide.

The reaction of compound of Formula XIX to give a compound of Formula XX can be carried out in presence of a base, for example, potassium carbonate, potassium bicarbonate, sodium carbonate, sodium bicarbonate, triethylamine, ammonium hydroxide, pyridine and 4-dimethylaminopyridine.

The reaction of compound of Formula XX with hydrochloric acid to give a compound of Formula XXI can be carried out in a solvent, for example, dichloromethane, chloroform, tetrahydrofuran, dichloroethane, benzene, xylene or isopropyl alcohol.

The reaction of compound of Formula XXI with a fluorinating agent to give a compound of Formula XXII can be carried out in a solvent for example, dichloromethane, tetrahydrofuran, dichloroethane, xylene, benzene, or toluene.

The reaction of compound XXI to give a compound of Formula XXII can be carried out in presence of a fluorinating agent, for example, diethyl amino sulfurtrifluoride or tris(dimethylamino)sulfur (trimethylsilyl)difluoride.

The reaction of compound XX to give a compound of Formula XXIII can be carried out in presence of a fluorinating agent, for example, diethyl amino sulfurtrifluoride or tris(dimethylamino)sulfur (trimethylsilyl)difluoride in a solvent, for example, toluene, xylene, benzene, dichloromethane, dichloroethane and tetrahydrofuran.

An illustrative list of compounds provided herein is given below (also shown in Table 1)

1-{3-[4-(5-Fluoro-2-propoxy-phenyl)-piperazin-1-yl]-propyl}-3-methyl-4-methylamino-pyrrolidine-2,5-dione (Compound No. 1)
1-{3-[4-(5-Fluoro-2-propoxy-phenyl)-piperazin-1-yl]-propyl}-3-methyl-4-methylamino-pyrrolidine-2,5-dione hydrochloride salt (Compound No. 2)
2-(3-{4-[2-(2,2,3,3-Tetrafluoro-propoxy)-phenyl]-piperazin-1-yl}-propyl)-hexahydro-isoindole-1, 3-dione (Compound No. 3)
2-(3-{4-[2-(2,2,3,3-Tetrafluoro-propoxy)-phenyl]-piperazin-1-yl}-propyl)-hexahydro-isoindole-1, 3-dione hydrochloride salt (Compound No. 4)
1-{3-[4-(5-Fluoro-2-propoxy-phenyl)-piperazin-1-yl]-propyl}-3-cyclopropylamino-4-methyl-pyrrolidine-2,5-dione (Compound No. 5)
1-{3-[4-(5-Fluoro-2-propoxy-phenyl)-piperazin-1-yl]-propyl}-3-cyclopropylamino-4-methyl-pyrrolidine-2,5-dione hydrochloride salt (Compound No. 6)
2-{3-[4-(2-Cyclopentyloxy-5-fluoro-phenyl)-piperazin-1-yl]-propyl}-5,6-difluoro-hexahydro-isoindole-1,3-dione (Compound No. 7)
2-{3-[4-(2-Cyclopentyloxy-5-fluoro-phenyl)-piperazin-1-yl]-propyl}-5,6-difluoro-hexahydro-isoindole-1,3-dione hydrochloride salt (Compound No. 8)
1-{3-[4-(2-Methoxy-5-methyl-phenyl)-piperazin-1-yl]-propyl}-3,4-dimethyl-pyrrolidine-2, 5-dione (Compound No. 9)
1-{(3-[4-(2-Methoxy-5-methyl-phenyl)-piperazin-1-yl]-propyl}-3,4-dimethyl-pyrrolidine-2, 5-dione hydrochloride salt (Compound No. 10)
1-{3-[4-(2-Methoxy-5-methyl-phenyl)-piperazin-1-yl]-propyl}-3-methyl-4-methylamino-pyrrolidine-2,5-dione (Compound No. 11)
1-{3-[4-(2-Methoxy-5-methyl-phenyl)-piperazin-1-yl]-propyl}-3-methyl-4-methylamino-pyrrolidine-2,5-dione hydrochloride salt (Compound No. 12)
1-{3-[4-(2-Methoxy-5-methyl-phenyl)-piperazin-1-yl]-propyl}-3-cyclopropylamino-4-methyl-pyrrolidine-2,5-dione (Compound No. 13)
1-{3-[4-(2-Methoxy-5-methyl-phenyl)-piperazin-1-yl]-propyl}-3-cyclopropylamino-4-methyl-pyrrolidine-2,5-dione hydrochloride salt (Compound No. 14)
1-{3-[4-(5-Fluoro-2-propoxy-phenyl)-piperazin-1-yl]-propyl}-3-cyclobutylamino-4-methyl-pyrrolidine-2,5-dione (Compound No. 15)
1-{3-[4-(5-Fluoro-2-propoxy-phenyl)-piperazin-1-yl]-propyl}-3-cyclobutylamino-4-methyl-pyrrolidine-2,5-dione hydrochloride salt (Compound No. 16)
1-(3-{4-[5-Fluoro-2-(2,2,2-trifluoro-ethoxy)-phenyl]-piperazin-1-yl}-propyl)-3-cyclopropylamino-4-methyl-pyrrolidine-2,5-dione (Compound No. 17)
1-(3-{4-[5-Fluoro-2-(2,2,2-trifluoro-ethoxy)-phenyl]-piperazin-1-yl}-propyl)-3-cyclopropylamino-4-methyl-pyrrolidine-2,5-dione hydrochloride salt (Compound No. 18)
1-(3-{4-[5-Fluoro-2-(2,2,2-trifluoro-ethoxy)-phenyl]-piperazin-1-yl}-propyl)-3-methyl-4-methylamino-pyrrolidine-2,5-dione (Compound No. 19)
1-(3-{4-[5-Fluoro-2-(2,2,2-trifluoro-ethoxy)-phenyl]-piperazin-1-yl}-propyl)-3-methyl-4-methylamino-pyrrolidine-2,5-dione hydrochloride salt (Compound No. 20)
1-(3-{4-[5-Fluoro-2-(2,2,2-trifluoro-ethoxy)-phenyl]-piperazin-1-yl}-propyl)-3,4-dimethyl-pyrrolidine-2,5-dione (Compound No. 21)
1-(3-{4-[5-Fluoro-2-(2,2,2-trifluoro-ethoxy)-phenyl]-piperazin-1-yl}-propyl)-3,4-dimethyl-pyrrolidine-2,5-dione hydrochloride salt (Compound No. 22)
1-(3-{4-[2-(2,2,3,3-Tetrafluoro-propoxy)-phenyl]-piperazin-1-yl}-propyl)-3,4-dimethyl-pyrrolidine-2,5-dione (Compound No. 23)
1-(3-{4-[2-(2,2,3,3-Tetrafluoro-propoxy)-phenyl]-piperazin-1-yl}-propyl)-3,4-dimethyl-pyrrolidine-2,5-dione hydrochloride salt (Compound No. 24)
1-(3-{4-[2-(2,2,3,3-Tetrafluoro-propoxy)-phenyl]-piperazin-1-yl}-propyl)-3-cyclopropylamino-4-methyl-pyrrolidine-2,5-dione (Compound No. 25)
1-(3-{4-[2-(2,2,3,3-Tetrafluoro-propoxy)-phenyl]-piperazin-1-yl}-propyl)-3-cyclopropylamino-4-methyl-pyrrolidine-2,5-dione hydrochloride salt (Compound No. 26)
1-(3-{4-[2-(2,2,3,3-Tetrafluoro-propoxy)-phenyl]-piperazin-1-yl}-propyl)-3-cyclobutylamino-4-methyl-pyrrolidine-2,5-dione (Compound No. 27)
1-(3-{4-[2-(2,2,3,3-Tetrafluoro-propoxy)-phenyl]-piperazin-1-yl}-propyl)-3-cyclobutylamino-4-methyl-pyrrolidine-2,5-dione hydrochloride salt (Compound No. 28)
1-{3-[4-(2-Cyclopentyloxy-phenyl)-piperazin-1-yl]-propyl}-3-cyclobutylamino-4-methyl-pyrrolidine-2,5-dione (Compound No. 29)
1-{3-[4-(2-Cyclopentyloxy-phenyl)-piperazin-1-yl]-propyl}-3-cyclobutylamino-4-methyl-pyrrolidine-2,5-dione hydrochloride salt (Compound No. 30)
2-{3-[4-(2-Cyclopentyloxy-5-fluoro-phenyl)-piperazin-1-yl]-propyl}-hexahydro-isoindole-1, 3-dione (Compound No. 31)
2-{3-[4-(2-Cyclopentyloxy-5-fluoro-phenyl)-piperazin-1-yl]-propyl}-hexahydro-isoindole-1, 3-dione hydrochloride salt (Compound No. 32)
1-{3-[4-(5-Fluoro-2-isopropoxy-phenyl)-piperazin-1-yl]-propyl}-3-methyl-4-methyl amino-pyrrolidine-2,5-dione (Compound No. 33)
1-{3-[4-(5-Fluoro-2-isopropoxy-phenyl)-piperazin-1-yl]-propyl}-3-methyl-4-methyl amino-pyrrolidine-2,5-dione hydrochloride salt (Compound No. 34)
1-{3-[4-(5-Fluoro-2-methoxy-phenyl)-piperazin-1-yl]-propyl}-3-methyl-4-methylamino-pyrrolidine-2,5-dione (Compound No. 35)
1-{3-[4-(5-Fluoro-2-methoxy-phenyl)-piperazin-1-yl]-propyl}-3-methyl-4-methylamino-pyrrolidine-2,5-dione hydrochloride salt (Compound No. 36)
1-{3-[4-(2-Cyclopropylmethoxy-phenyl)-piperazin-1-yl]-propyl}-3,4-dimethyl-pyrrolidine-2, 5-dione (Compound No. 37)
1-{3-[4-(2-Cyclopropylmethoxy-phenyl)-piperazin-1-yl]-propyl}-3,4-dimethyl-pyrrolidine-2, 5-dione hydrochloride salt (Compound No. 38)
2-{3-[4-(5-Fluoro-2-isopropoxy-phenyl)-piperazin-1-yl]-propyl}-hexahydro-isoindole-1, 3-dione (Compound No. 39)
2-{3-[4-(5-Fluoro-2-isopropoxy-phenyl)-piperazin-1-yl]-propyl}-hexahydro-isoindole-1, 3-dione hydrochloride salt (Compound No. 40)
2-{3-[4-(2-Cyclopentyloxy-5-fluoro-phenyl)-piperazin-1-yl]-propyl}-3a,4,7,7a-tetrahydro-isoindole-1,3-dione (Compound No. 41)
2-{3-[4-(2-Cyclopentyloxy-5-fluoro-phenyl)-piperazin-1-yl]-propyl}-3a,4,7,7a-tetrahydro-isoindole-1,3-dione hydrochloride salt (Compound No. 42)
1-{3-[4-(2-Cyclopropylmethoxy-phenyl)-piperazin-1-yl]-propyl}-3-cyclopropylamino-4-methyl-pyrrolidine-2,5-dione (Compound No. 43)
1-{3-[4-(2-Cyclopropylmethoxy-phenyl)-piperazin-1-yl]-propyl}-3-cyclopropylamino-4-methyl-pyrrolidine-2,5-dione hydrochloride salt (Compound No. 44)
2-{3-[4-(5-Fluoro-2-isopropoxy-phenyl)-piperazin-1-yl]-propyl}-3a,4,7,7a-tetrahydro-isoindole-1,3-dione (Compound No. 45)
2-{3-[4-(5-Fluoro-2-isopropoxy-phenyl)-piperazin-1-yl]-propyl}-3a,4,7,7a-tetrahydro-isoindole-1,3-dione hydrochloride salt (Compound No. 46)
1-{3-[4-(5-Fluoro-2-isopropoxy-phenyl)-piperazin-1-yl]-propyl}-3-cyclobutylamino-4-methyl-pyrrolidin-2,5-dione (Compound No. 47)
1-{3-[4-(5-Fluoro-2-isopropoxy-phenyl)-piperazin-1-yl]-propyl}-3-cyclobutylamino-4-methyl-pyrrolidin-2,5-dione hydrochloride salt (Compound No. 48)
1-{3-[4-(3-Fluoro-2-isopropoxy-phenyl)-piperazin-1-yl]-propyl}-3-methyl-4-methylamino-pyrrolidine-2,5-dione (Compound No. 49)
1-{3-[4-(3-Fluoro-2-isopropoxy-phenyl)-piperazin-1-yl)-propyl}-3-methyl-4-methylamino-pyrrolidine-2,5-dione hydrochloride salt (Compound No. 50)
1-{3-[4-(2-Cyclopropylmethoxy-phenyl)-piperazin-1-yl]-propyl}-3-isopropylamino-4-methyl-pyrrolidine-2,5-dione (Compound No. 51)
1-{3-[4-(2-Cyclopropylmethoxy-phenyl)-piperazin-1-yl]-propyl}-3-isopropylamino-4-methyl-pyrrolidine-2,5-dione hydrochloride salt (Compound No. 52)
1-{3-[4-(2-Cyclopentyloxy-5-fluoro-phenyl)-piperazin-1-yl]-propyl}-3,4-dimethyl-pyrrolidine-2, 5-dione (Compound No. 53)
1-{3-[4-(2-Cyclopentyloxy-5-fluoro-phenyl)-piperazin-1-yl]-propyl}-3,4-dimethyl-pyrrolidine-2, 5-dione hydrochloride salt (Compound No. 54)
1-{3-[4-(2-Cyclopentyloxy-5-fluoro-phenyl)-piperazin-1-yl]-propyl}-3-cyclopropylamino-4-methyl-pyrrolidine-2,5-dione (Compound No. 55)
1-{3-[4-(2-Cyclopentyloxy-5-fluoro-phenyl)-piperazin-1-yl]-propyl}-3-cyclopropylamino-4-methyl-pyrrolidine-2,5-dione hydrochloride salt (Compound No. 56)
1-{3-[4-(5-Fluoro-2-methoxy-phenyl)-piperazin-1-yl]-propyl}-3-(cyclopropylmethyl-amino)-4-methyl-pyrrolidine-2,5-dione (Compound No. 57)
1-{3-[4-(5-Fluoro-2-methoxy-phenyl)-piperazin-1-yl]-propyl}-3-(cyclopropylmethyl-amino)-4-methyl-pyrrolidine-2,5-dione hydrochloride salt (Compound No. 58)
1-{3-[4-(5-Fluoro-2-isopropoxy-phenyl)-piperazin-1-yl]-propyl}-3-(cyclopropylmethyl-amino)-4-methyl-pyrrolidine-2,5-dione (Compound No. 59)
1-{3-[4-(5-Fluoro-2-isopropoxy-phenyl)-piperazin-1-yl]-propyl}-3-(cyclopropylmethyl-amino)-4-methyl-pyrrolidine-2,5-dione hydrochloride salt (Compound No. 60)
1-{3-[4-(5-Fluoro-2-trifluoromethoxy-phenyl)-piperazin-1-yl]-propyl}-3-cyclopropylamino-4-methyl-pyrrolidine-2,5-dione (Compound No. 61)
1-{3-[4-(5-Fluoro-2-trifluoromethoxy-phenyl)-piperazin-1-yl]-propyl}-3-cyclopropylamino-4-methyl-pyrrolidine-2,5-dione hydrochloride salt (Compound No. 62)
1-{3-[4-(2-Methoxy-phenyl)-piperazin-1-yl]-propyl}-3-(cyclopropylmethyl-amino)-4-methyl-pyrrolidine-2,5-dione (Compound No. 63)
1-{3-[4-(2-Methoxy-phenyl)-piperazin-1-yl]-propyl}-3-(cyclopropylmethyl-amino)-4-methyl-pyrrolidine-2,5-dione hydrochloride salt (Compound No. 64)
1-{3-[4-(2-Isopropoxy-phenyl)-piperazin-1-yl]-propyl}-3-isopropylamino-4-methyl-pyrrolidine-2, 5-dione (Compound No. 65)
1-{3-[4-(2-Isopropoxy-phenyl)-piperazin-1-yl)-propyl}-3-isopropylamino-4-methyl-pyrrolidine-2,5-dione hydrochloride salt (Compound No. 66)
1-{3-[4-(2-Isopropoxy-phenyl)-piperazin-1-yl]-propyl}-3-cyclopropylamino-4-methyl-pyrrolidine-2, 5-dione (Compound No. 67)
1-{3-[4-(2-Isopropoxy-phenyl)-piperazin-1-yl]-propyl}-3-cyclopropylamino-4-methyl-pyrrolidine-2, 5-dione hydrochloride salt (Compound No. 68)
1-{3-[4-(2-Methoxy-phenyl)-piperazin-1-yl]-propyl}-3-isopropylamino-4-methyl-pyrrolidine-2, 5-dione (Compound No. 69)
1-{3-[4-(2-Methoxy-phenyl)-piperazin-1-yl]-propyl}-3-isopropylamino-4-methyl-pyrrolidine-2, 5-dione hydrochloride salt (Compound No. 70)
1-{3-[4-(5-Fluoro-2-isopropoxy-phenyl)-piperazin-1-yl]-propyl}-3,4-dimethyl-pyrrole-2, 5-dione (Compound No. 71)
1-{3-[4-(5-Fluoro-2-isopropoxy-phenyl)-piperazin-1-yl]-propyl}-3,4-dimethyl-pyrrole-2, 5-dione hydrochloride salt (Compound No. 72)
1-{3-[4-(5-Fluoro-2-isopropoxy-phenyl)-piperazin-1-yl]-propyl}-3,4-dimethyl-pyrrolidine-2,5-dione (Compound No. 73)
1-{3-[4-(5-Fluoro-2-isopropoxy-phenyl)-piperazin-1-yl]-propyl}-3,4-dimethyl-pyrrolidine-2,5-dione hydrochloride salt (Compound No. 74)
1-{3-[4-(5-Fluoro-2-isopropoxy-phenyl)-piperazin-1-yl]-propyl}-3-cyclopropylamino-4-methyl-pyrrolidine-2,5-dione (Compound No. 75)
1-{3-[4-(5-Fluoro-2-isopropoxy-phenyl)-]-piperazin-1-yl]-propyl}-3-cyclopropylamino-4-methyl-pyrrolidine-2,5-dione hydrochloride salt (Compound No. 76)
1-{3-[4-(5-Fluoro-2-isopropoxy-phenyl)-piperazin-1-yl]-propyl}-3-isopropylamino-4-methyl-pyrrolidine-2,5-dione (Compound No. 77)
1-{3-[4-(5-Fluoro-2-isopropoxy-phenyl)-piperazin-1-yl]-propyl}-3-isopropylamino-4-methyl-pyrrolidine-2,5-dione hydrochloride salt (Compound No. 78)
1-{3-[4-(5-Fluoro-2-isopropoxy-phenyl)-piperazin-1-yl]-propyl}-3-(cyclopropyl-methyl-amino)-4-methyl-pyrrolidine-2,5-dione (Compound No. 79)
1-{3-[4-(5-Fluoro-2-isopropoxy-phenyl)-piperazin-1-yl]-propyl}-3-(cyclopropyl-methyl-amino)-4-methyl-pyrrolidine-2,5-dione hydrochloride salt (Compound No. 80)
1-(3-{4-[2-(2,2,2-Trifluoro-ethoxy)-phenyl]-piperazin-1-yl}-propyl)-3,4-dimethyl-pyrrole-2,5-dione (Compound No. 81)
1-(3-{4-[2-(2,2,2-Trifluoro-ethoxy)-phenyl]-piperazin-1-yl}-propyl)-3,4-dimethyl-pyrrole-2,5-dione hydrochloride salt (Compound No. 82)
1-(3-{4-[2-(2,2,2-Trifluoro-ethoxy)-phenyl]-piperazin-1-yl}-propyl)-3,4-dimethyl-pyrrolidine-2,5-dione (Compound No. 83)
1-(3-{4-[2-(2,2,2-Trifluoro-ethoxy)-phenyl]-piperazin-1-yl}-propyl)-3,4-dimethyl-pyrrolidine-2,5-dione hydrochloride salt (Compound No. 84)
1-{3-[4-(2-Cyclopentyloxy-phenyl)-piperazin-1-yl]-propyl}-3,4-dimethyl-pyrrole-2,5-dione (Compound No. 85)
1-{3-[4-(2-Cyclopentyloxy-phenyl)-piperazin-1-yl]-propyl}-3,4-dimethyl-pyrrole-2,5-dione hydrochloride salt (Compound No. 86)
1-{3-[4-(2-Cyclopentyloxy-phenyl)-piperazin-1-yl]-propyl}-3,4-dimethyl-pyrrolidine-2,5-dione (Compound No. 87)
1-{3-[4-(2-Cyclopentyloxy-phenyl)-piperazin-1-yl]-propyl}-3,4-dimethyl-pyrrolidine-2,5-dione hydrochloride salt (Compound No. 88)
1-{3-[4-(2-Cyclopentyloxy-phenyl)-piperazin-1-yl]-propyl}-3-isopropylamino-4-methyl-pyrrolidine-2, 5-dione (Compound No. 89)
1-{3-[4-(2-Cyclopentyloxy-phenyl)-piperazin-1-yl]-propyl}-3-isopropylamino-4-methyl-pyrrolidine-2, 5-dione hydrochloride salt (Compound No. 90)
1-{3-[4-(5-Fluoro-2-methoxy-phenyl)-piperazin-1-yl]-propyl}-3-cyclopropylamino-4-methyl-pyrrolidine-2,5-dione (Compound No. 91)
1-{3-[4-(5-Fluoro-2-methoxy-phenyl)-piperazin-1-yl]-propyl}-3-cyclopropylamino-4-methyl-pyrrolidine-2,5-dione hydrochloride salt (Compound No. 92)
1-{3-[4-(2-Cyclopentyloxy-phenyl)-piperazin-1-yl]-propyl}-3-methyl-4-methylamino-pyrrolidine-2,5-dione (Compound No. 93)
1-{3-[4-(2-Cyclopentyloxy-phenyl)-piperazin-1-yl]-propyl}-3-methyl-4-methylamino-pyrrolidine-2,5-dione hydrochloride salt (Compound No. 94)
1-{3-[4-(5-Fluoro-2-methoxy-phenyl)-piperazin-1-yl]-propyl}-3,4-dimethyl-pyrrolidine-2, 5-dione (Compound No. 95)
1-{3-[4-(5-Fluoro-2-methoxy-phenyl)-piperazin-1-yl]-propyl}-3,4-dimethyl-pyrrolidine-2, 5-dione hydrochloride salt (Compound No. 96)
1-{3-[4-(3-Fluoro-2-methoxy-phenyl)-piperazin-1-yl]-propyl}-3,4-dimethyl-pyrrole-2,5-dione (Compound No. 97)
1-{3-[4-(3-Fluoro-2-methoxy-phenyl)-piperazin-1-yl)-propyl}-3,4-dimethyl-pyrrole-2,5-dione hydrochloride salt (Compound No. 98)
1-{3-[4-(3-Fluoro-2-methoxy-phenyl)-piperazin-1-yl]-propyl}-3,4-dimethyl-pyrrolidine-2,5-dione (Compound No. 99)
1-{3-[4-(3-Fluoro-2-methoxy-phenyl)-piperazin-1-yl]-propyl}-3,4-dimethyl-pyrrolidine-2,5-dione hydrochloride salt (Compound No. 100)
1-{3-[4-(3-Fluoro-2-methoxy-phenyl)-piperazin-1-yl]-propyl}-3-methyl-4-methylamino-pyrrolidine-2,5-dione (Compound No. 101)
1-{3-[4-(3-Fluoro-2-methoxy-phenyl)-piperazin-1-yl]-propyl}-3-methyl-4-methylamino-pyrrolidine-2,5-dione hydrochloride salt (Compound No. 102)
1-{3-[4-(3-Fluoro-2-methoxy-phenyl)-piperazin-1-yl]-propyl}-3-cyclopropylamino-4-methyl-pyrrolidine-2,5-dione (Compound No. 103)
1-{3-[4-(3-Fluoro-2-methoxy-phenyl)-piperazin-1-yl]-propyl}-3-cyclopropylamino-4-methyl-pyrrolidine-2,5-dione hydrochloride salt (Compound No. 104)
1-{3-[4-(3-Fluoro-2-isopropoxy-phenyl)-piperazin-1-yl]-propyl}-3,4-dimethyl-pyrrolidine-2, 5-dione (Compound No. 105)
1-{3-[4-(3-Fluoro-2-isopropoxy-phenyl)-piperazin-1-yl]-propyl}-3,4-dimethyl-pyrrolidine-2, 5-dione hydrochloride salt (Compound No. 106)
1-{3-[4-(3-Fluoro-2-isopropoxy-phenyl)-piperazin-1-yl]-propyl}-3-cyclopropylamino-4-methyl-pyrrolidine-2,5-dione (Compound No. 107)
1-{3-[4-(3-Fluoro-2-isopropoxy-phenyl)-piperazin-1-yl]-propyl}-3-cyclopropylamino-4-methyl-pyrrolidine-2,5-dione hydrochloride salt (Compound No. 108)
2-(3-{4-(2-(2,2,3,3-Tetrafluoro-propoxy)-phenyl]-piperazin-1-yl}-propyl)-3a,4,7,7a-tetrahydro-isoindole-1,3-dione (Compound No. 109)
2-(3-{4-[2-(2,2,3,3-Tetrafluoro-propoxy)-)-phenyl]-piperazin-1-yl}-propyl)-3a,4,7,7a-tetrahydro-isoindole-1,3-dione hydrochloride salt (Compound No. 110)
2-{4-[4-[2-Isopropoxy-phenyl]-piperazin-1-yl]-butyl}-3a,4,7,7a-tetrahydro-isoindole-1, 3-dione (Compound No. 111)
2-{4-[4-[2-Isopropoxy-phenyl]-piperazin-1-yl]-butyl}-3a,4,7,7a-tetrahydro-isoindole-1, 3-dione hydrochloride salt (Compound No. 112)
2-{3-[4-(4-Fluoro-2-methoxy-phenyl)-piperazin-1-yl]-propyl}-3a,4,7,7a-tetrahydro-isoindole-1, 3-dione (Compound No. 113)
2-{3-[4-(4-Fluoro-2-methoxy-phenyl)-piperazin-1-yl]-propyl}-3a,4,7,7a-tetrahydro-isoindole-1, 3-dione hydrochloride salt (Compound No. 114)
2-(3-{-[4-Fluoro-2-(2,2,2-trifluoro-ethoxy)-phenyl]-piperazin-1-yl}-propyl)-3a,4,7,7a-tetrahydro-isoindole-1,3-dione (Compound No. 115)
2-(3-{4-[4-Fluoro-2-(2,2,2-trifluoro-ethoxy)-phenyl]-piperazin-1-yl}-propyl)-3a,4,7,7a-tetrahydro-isoindole-1, 3-dione hydrochloride salt (Compound No. 116)
2-{3-[4-(4-Fluoro-2-isopropoxy-phenyl)-piperazin-1-yl]-propyl}-3a,4,7,7a-tetrahydro-isoindole-1, 3-dione (Compound No. 117)
2-{3-[4-(4-Fluoro-2-isopropoxy-phenyl)-piperazin-1-yl]-propyl}-3a,4,7,7a-tetrahydro-isoindole-1, 3-dione hydrochloride salt (Compound No. 118)
2-{3-[4-(2-Isopropoxy-phenyl)-piperazin-1-yl]-propyl}-5-chloro-6-fluoro-hexahydro-isoindole-1, 3-dione (Compound No. 119)
2-{3-[4-(2-Isopropoxy-phenyl)-piperazin-1-yl]-propyl}-5-chloro-6-fluoro-hexahydro-isoindole-1, 3-dione hydrochloride salt (Compound No. 120)
1-{3-[4-(5-Fluoro-2-propoxy-phenyl)-piperazin-1-yl]-propyl}-3-methyl-pyrrolidine-2,5-dione (Compound No. 121)
1-{3-[4-(5-Fluoro-2-propoxy-phenyl)-piperazin-1-yl]-propyl}-3-methyl-pyrrolidine-2,5-dione hydrochloride salt (Compound No. 122)
1-{3-[4-(5-Fluoro-2-propoxy-phenyl)-piperazin-1-yl]-propyl}-3-cyclopropylamino-methyl-pyrrolidine-2,5-dione (Compound No. 123)
1-{3-[4-(5-Fluoro-2-propoxy-phenyl)-piperazin-1-yl]-propyl}-3-cyclopropylamino-methyl-pyrrolidine-2,5-dione hydrochloride salt (Compound No. 124)
1-{3-[4-(2-methoxy-5-methyl-phenyl)-piperazin-1-yl]-propyl}-3-cyclopropylamino methyl-pyrrolidine-2,5-dione (Compound No. 125)
1-{3-[4-(2-methoxy-5-methyl-phenyl)-piperazin-1-yl]-propyl}-3-cyclopropylamino methyl-pyrrolidine-2,5-dione hydrochloride salt (Compound No. 126)
1-(3-{4-[5-Fluoro-2-(2,2,2-trifluoro-ethoxy)-phenyl]-piperazin-1-yl}-propyl)-3-cyclopropylaminomethyl-pyrrolidine-2,5-dione (Compound No. 127)
1-(3-{4-[5-Fluoro-2-(2,2,2-trifluoro-ethoxy)-phenyl]-piperazin-1-yl}-propyl)-3-cyclopropylaminomethyl-pyrrolidine-2,5-dione hydrochloride salt (Compound No. 128)
1-(3-{4-[2-(2,2,3,3-Tetrafluoro-propoxy)-phenyl)-piperazin-1-yl}-propyl)-3-cyclopropylaminomethyl-pyrrolidine-2,5-dione (Compound No. 129)
1-(3-{4-[2,2,3,3-Tetrafluoro-propoxy)-phenyl]-piperazin-1-yl}-propyl)-3-cyclopropylaminomethyl-pyrrolidine-2,5-dione hydrochloride salt (Compound No. 130)
1-(3-{4-[2-(2,2,3,3-Tetrafluoro-propoxy)-phenyl]-piperazin-1-yl}-propyl)-3-cyclobutylamino-methyl-pyrrolidine-2,5-dione (Compound No. 131)
1-(3-{4-[2-(2,2,3,3-Tetrafluoro-propoxy)-phenyl]-piperazin-1-yl}-propyl)-3-cyclobutylamino-methyl-pyrrolidine-2,5-dione hydrochloride salt (Compound No. 132)
1-{3-[4-(3-Fluoro-2-methoxy-phenyl)-piperazin-1-yl]-propyl}-3-methyl-pyrrolidine-2,5-dione (Compound No. 133)
1-{3-[4-(3-Fluoro-2-methoxy-phenyl)-piperazin-1-yl]-propyl}-3-methyl-pyrrolidine-2,5-dione hydrochloride salt (Compound No. 134)
5-{3-[4-(5-Fluoro-2-isopropoxy-phenyl)-piperazin-1-yl]-propyl}-5-aza-spiro[2.4]heptane-4,6-dione (Compound No. 135)
5-{3-[4-(5-Fluoro-2-isopropoxy-phenyl)-piperazin-1-yl]-propyl}-5-aza-spiro[2.4]heptane-4,6-dione hydrochloride salt (Compound No. 136)
1-{3-[4-(5-Fluoro-2-isopropoxy-phenyl)-piperazin-1-yl]-propyl}-3-methylaminomethyl-pyrrolidine-2,5-dione (Compound No. 137)
1-{3-[4-(5-Fluoro-2-isopropoxy-phenyl)-piperazin-1-yl]-propyl}-3-methylaminomethyl-pyrrolidine-2,5-dione hydrochloride salt (Compound No. 138)
1-{3-[4-(2-Cyclopropylmethoxy-phenyl)-piperazin-1-yl]-propyl}-3-cyclopropylamino-methyl-pyrrolidine-2,5-dione (Compound No. 139)
1-{3-[4-(2-Cyclopropylmethoxy-phenyl)-piperazin-1-yl]-propyl}-3-cyclopropylamino-methyl-pyrrolidine-2,5-dione hydrochloride salt (Compound No. 140)
1-{3-[4-(2-Cyclopropylmethoxy-phenyl)-piperazin-1-yl]-propyl}-3-methylamino-methyl-pyrrolidine-2, 5-dione (Compound No. 141)
1-{3-[4-(2-Cyclopropylmethoxy-phenyl)-piperazin-1-yl]-propyl}-3-methylamino-methyl-pyrrolidine-2, 5-dione hydrochloride salt (Compound No. 142)
1-{3-[4-(2-Cyclopropylmethoxy-phenyl)-piperazin-1-yl]-propyl}-3-methyl-pyrrolidine-2, 5-dione (Compound No. 143)
1-{3-[4-(2-Cyclopropylmethoxy-phenyl)-piperazin-1-yl]-propyl}-3-methyl-pyrrolidine-2, 5-dione hydrochloride salt (Compound No. 144)
1-{3-[4-(2-Methoxy-phenyl)-piperazin-1-yl]-propyl}-3-methylaminomethyl-pyrrolidine-2, 5-dione (Compound No. 145)
1-{3-[4-(2-Methoxy-phenyl)-piperazin-1-yl]-propyl}-3-methylaminomethyl-pyrrolidine-2, 5-dione hydrochloride salt (Compound No. 146)
1-{3-[4-(2-Cyclopentyloxy-phenyl)-piperazin-1-yl]-propyl}-3-cyclopropylaminomethyl-pyrrolidine-2, 5-dione (Compound No. 147)
1-{3-[4-(2-Cyclopentyloxy-phenyl)-piperazin-1-yl]-propyl}-3-cyclopropylaminomethyl-pyrrolidine-2,5-dione hydrochloride salt (Compound No. 148)
1-{3-[4-(2-Cyclopentyloxy-phenyl)-piperazin-1-yl]-propyl}-3-(isopropylamino-methyl)-pyrrolidine-2,5-dione (Compound No. 149)
1-{3-[4-(2-Cyclopentyloxy-phenyl)-piperazin-1-yl]-propyl}-3-(isopropylamino-methyl)-pyrrolidine-2,5-dione hydrochloride salt (Compound No. 150)
5-{3-[4-(2-Cyclopentyloxy-phenyl)-piperazin-1-yl]-propyl}-5-aza-spiro[2.4]heptane-4, 6-dione (Compound No. 151)
5-{3-[4-(2-Cyclopentyloxy-phenyl)-piperazin-1-yl]-propyl}-5-aza-spiro[2.4]heptane-4,6-dione hydrochloride salt (Compound No. 152)
1-{3-[4-(3-Fluoro-2-isopropoxy-phenyl)-piperazin-1-yl]-propyl}-3-(isopropylamino-methyl)-pyrrolidine-2, 5-dione (Compound No. 153)
1-{3-[4-(3-Fluoro-2-isopropoxy-phenyl)-piperazin-1-yl]-propyl}-3-(isopropylamino-methyl)-pyrrolidine-2, 5-dione hydrochloride salt (Compound No. 154)
1-{3-[4-(3-Fluoro-2-isopropoxy-phenyl)-piperazin-1-yl]-propyl}-3-cyclopropylamino-methyl-pyrrolidine-2, 5-dione (Compound No. 155)
1-{3-[4-(3-Fluoro-2-isopropoxy-phenyl)-piperazin-1-yl]-propyl}-3-cyclopropylamino-methyl-pyrrolidine-2, 5-dione hydrochloride salt (Compound No. 156)
5-{3-[4-(3-Fluoro-2-isopropoxy-phenyl)-piperazin-1-yl]-propyl}-5-aza-spiro[2.4]heptane-4, 6-dione (Compound No. 157)
5-{3-[4-(3-Fluoro-2-isopropoxy-phenyl)-piperazin-1-yl]-propyl}-5-aza-spiro[2,4]heptane-4,6-dione hydrochloride salt (Compound No. 158)
1-{3-[4-(5-Fluoro-2-isopropoxy-phenyl)-piperazin-1-yl]-propyl}-3-cyclopropylaminomethyl-pyrrolidine-2,5-dione (Compound No. 159)
1-{3-[4-(5-Fluoro-2-isopropoxy-phenyl)-piperazin-1-yl]-propyl}-3-cyclopropylamino-methyl-pyrrolidine-2, 5-dione hydrochloride salt (Compound No. 160)
1-{3-[4-(5-Fluoro-2-isopropoxy-phenyl)-piperazin-1-yl]-propyl}-3-(isopropylamino-methyl)-pyrrolidine-2, 5-dione (Compound No. 161)
1-{3-[4-(5-Fluoro-2-isopropoxy-phenyl)-piperazin-1-yl]-propyl}-3-(isopropylamino-methyl)-pyrrolidine-2, 5-dione hydrochloride salt (Compound No. 162)
1-{3-[4-(2-Cyclopentyloxy-5-fluoro-phenyl)-piperazin-1-yl]-propyl}-3-[(cyclopropyl-methyl-amino)-methyl]-pyrrolidine-2,5-dione (Compound No. 163)
1-{3-[4-(2-Cyclopentyloxy-5-fluoro-phenyl)-piperazin-1-yl]-propyl}-3-[(cyclopropyl-methyl-amino)-methyl]-pyrrolidine-2,5-dione hydrochloride salt (Compound No. 164)
1-{3-[4-(2-Cyclopentyloxy-5-fluoro-phenyl)-piperazin-1-yl]-propyl}-3-isopropylamino-methyl)-pyrrolidine-2,5-dione (Compound No. 165)
1-{3-[4-(2-Cyclopentyloxy-5-fluoro-phenyl)-piperazin-1-yl]-propyl}-3-isopropylamino-methyl)-pyrrolidine-2,5-dione hydrochloride salt (Compound No. 166)
1-{3-[4-(2-Cyclopentyloxy-5-fluoro-phenyl)-piperazin-1-yl]-propyl}-3-cyclopropylaminomethyl-pyrrolidine-2,5-dione (Compound No. 167)
1-{3-[4-(2-Cyclopentyloxy-5-fluoro-phenyl)-piperazin-1-yl]-propyl}-3-cyclopropylaminomethyl-pyrrolidine-2,5-dione hydrochloride salt (Compound No. 168)
1-{3-[4-(2-Cyclopentyloxy-5-fluoro-phenyl)-piperazin-1-yl]-propyl}-3-methyl-pyrrolidine-2, 5-dione (Compound No. 169)
1-{3-[4-(2-Cyclopentyloxy-5-fluoro-phenyl)-piperazin-1-yl]-propyl}-3-methyl-pyrrolidine-2, 5-dione hydrochloride salt (Compound No. 170)
1-{3-[4-(2-Cyclopentyloxy-5-fluoro-phenyl)-piperazin-1-yl]-propyl}-3-methyl-pyrrole-2, 5-dione (Compound No. 171)
1-{3-[4-(2-Cyclopentyloxy-5-fluoro-phenyl)-piperazin-1-yl]-propyl}-3-methyl-pyrrole-2, 5-dione hydrochloride salt (Compound No. 172)
1-{3-[4-(2-Methoxy-phenyl)-piperazin-1-yl]-propyl}-3-methyl-4-prop-2-ynylamino-pyrrolidine-2, 5-dione (Compound No. 173)
1-{3-[4-(2-Methoxy-phenyl)-piperazin-1-yl]-propyl}-3-methyl-4-prop-2-ynylamino-pyrrolidine-2, 5-dione hydrochloride salt (Compound No. 174)
1-{3-[4-(4-Fluoro-2-isopropoxy-phenyl)-piperazin-1-yl]-propyl}-3-[(cyclopropyl-methyl-amino)-methyl]-pyrrolidine-2,5-dione (Compound No. 175)
1-{3-[4-(4-Fluoro-2-isopropoxy-phenyl)-piperazin-1-yl]-propyl}-3-[(cyclopropyl-methyl-amino)-methyl]-pyrrolidine-2,5-dione hydrochloride salt (Compound No. 176)
1-{3-[4-(2-Methoxy-phenyl)-piperazin-1-yl]-propyl}-3-[(cyclopropyl-methyl-amino)-methyl]-pyrrolidine-2,5-dione (Compound No. 177)
1-{3-[4-(2-Methoxy-phenyl)-piperazin-1-yl]-propyl}-3-[(cyclopropyl-methyl-amino)-methyl]-pyrrolidine-2,5-dione hydrochloride salt (Compound No. 178)
1-(3-{4-(2-(2,2,2-Trifluoro-ethoxy)-phenyl]-piperazin-1-yl}-propyl)-3-[(cyclopropyl-methyl-amino)-methyl]-pyrrolidine-2,5-dione (Compound No. 179)
1-(3-{4-(2-(2,2,2-Trifluoro-ethoxy)-phenyl]-piperazin-1-yl}-propyl)-3-[(cyclopropyl-methyl-amino)-methyl]-pyrrolidine-2,5-dione hydrochloride salt (Compound No. 180)
1-{3-[4-(2-Methoxy-phenyl)-piperazin-1-yl]-propyl}-3-(isopropylamino)-methyl)-pyrroline-2,5-dione (Compound No. 181)
1-{3-[4-(2-Methoxy-phenyl)-piperazin-1-yl]-propyl}-3-(isopropylamino)-methyl)-pyrrolidine-2,5-dione hydrochloride salt (Compound No. 182)
5-(3-{4-[2-(2,2,2-Trifluoro-ethoxy)-phenyl]-piperazin-1-yl}-propyl)-5-aza-spiro[2,4]heptane-4,6-dione (Compound No. 183)
5-(3-{4-[2-(2,2,2-Trifluoro-ethoxy)-phenyl]-piperazin-1-yl}-propyl)-5-aza-spiro[2,4]heptane-4,6-dione hydrochloride salt (Compound No. 184)
1-{3-[4-(5-Fluoro-2-isopropoxy-phenyl)-piperazin-1-yl]-propyl}-3-methyl-pyrrole-2,5-dione (Compound No. 185)
1-{3-[4-(5-Fluoro-2-isopropoxy-phenyl)-piperazin-1-yl]-propyl}-3-methyl-pyrrole-2,5-dione hydrochloride salt (Compound No. 186)
1-{3-[4-(5-Fluoro-2-isopropoxy-phenyl)-piperazin-1-yl]-propyl}-3-methyl-pyrrolidine-2,5-dione (Compound No. 187)
1-{3-[4-(5-Fluoro-2-isopropoxy-phenyl)-piperazin-1-yl]-propyl}-3-methyl-pyrrolidine-2,5-dione hydrochloride salt (Compound No. 188)
1-{3-[4-(5-Fluoro-2-methoxy-phenyl)-piperazin-1-yl]-propyl}-3-methyl-pyrrole-2,5-dione (Compound No. 189)
1-{3-[4-(5-Fluoro-2-methoxy-phenyl)-piperazin-1-yl]-propyl}-3-methyl-pyrrole-2,5-dione hydrochloride salt (Compound No. 190)
1-{3-[4-(5-Fluoro-2-methoxy-phenyl)-piperazin-1-yl]-propyl}-3-methyl-pyrrolidine-2,5-dione (Compound No. 191)
1-{3-[4-(5-Fluoro-2-methoxy-phenyl)-piperazin-1-yl]-propyl}-3-methyl-pyrrolidine-2,5-dione hydrochloride salt (Compound No. 192)
1-{3-[4-(2-Cyclopentyloxy-phenyl)-piperazin-1-yl]-propyl}-3-methyl-pyrrolidine-2,5-dione (Compound No. 193)
1-{3-[4-(2-Cyclopentyloxy-phenyl)-piperazin-1-yl]-propyl}-3-methyl-pyrrolidine-2,5-dione hydrochloride salt (Compound No. 194)
1-{3-[4-(3-Fluoro-2-methoxy-phenyl)-piperazin-1-yl]-propyl}-3-methyl-pyrrole-2,5-dione (Compound No. 195)
1-{3-[4-(3-Fluoro-2-methoxy-phenyl)-piperazin-1-yl]-propyl}-3-methyl-pyrrole-2,5-dione hydrochloride salt (Compound No. 196)
1-{3-[4-(3-Fluoro-2-isopropoxy-phenyl)-piperazin-1-yl]-propyl}-3-methyl-pyrrolidine-2,5-dione (Compound No. 197)

1-{3-[4-(3-Fluoro-2-isopropoxy-phenyl)-piperazin-1-yl]-propyl}-3-methyl-pyrrolidine-2,5-dione hydrochloride salt (Compound No. 198)

TABLE I

| | |
|---|---|
| | Formula I |
| 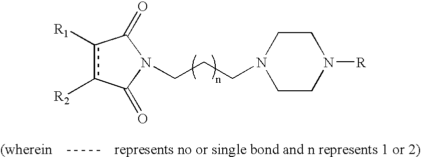 | |

| Compound No. | R₁ | R₂ | R |
|---|---|---|---|
| 1 | —CH₃ | —NHCH₃ | 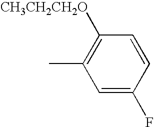 |
| 2 (HCl salt) | —CH₃ | —NHCH₃ | 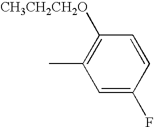 |
| 3 | 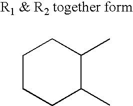 | | 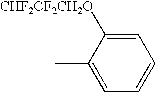 |
| 4 (HCl salt) | 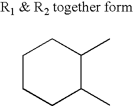 | | 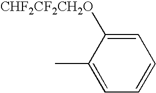 |
| 5 | —CH₃ | 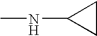 | 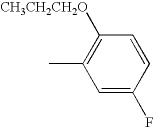 |
| 6 (HCl salt) | —CH₃ | 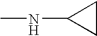 | 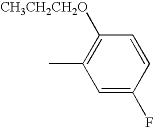 |
| 7 | 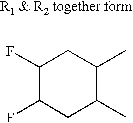 | | 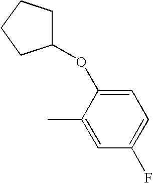 |
| 8 (HCl salt) | 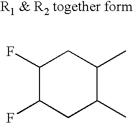 | | 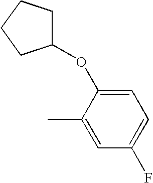 |
| 9 | —CH₃ | —CH₃ | 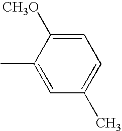 |
| 10 (HCl salt) | —CH₃ | —CH₃ | 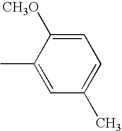 |
| 11 | —CH₃ | —NHCH₃ | 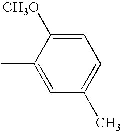 |
| 12 (HCl salt) | —CH₃ | —NHCH₃ | 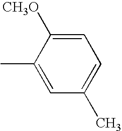 |
| 13 | —CH₃ | 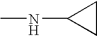 | 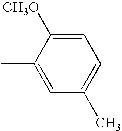 |
| 14 (HCl salt) | —CH₃ | 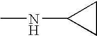 | 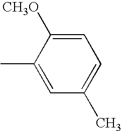 |
| 15 | —CH₃ | 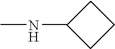 | 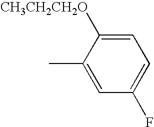 |
| 16 (HCl salt) | —CH₃ | 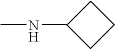 | 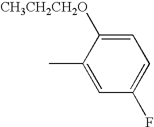 |
| 17 | —CH₃ | 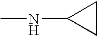 | 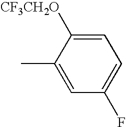 |
| 18 (HCl salt) | —CH₃ | 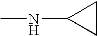 | 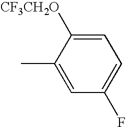 |
| 19 | —CH₃ | —NHCH₃ | 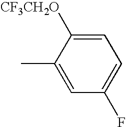 |
| 20 (HCl salt) | —CH₃ | —NHCH₃ | 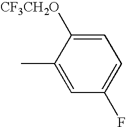 |
| 21 | —CH₃ | —CH₃ | 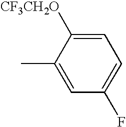 |
| 22 (HCl salt) | —CH₃ | —CH₃ | 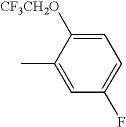 |
| 23 | —CH₃ | —CH₃ | 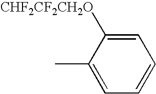 |
| 24 (HCl salt) | —CH₃ | —CH₃ | 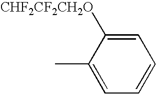 |
| 25 | —CH₃ | 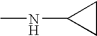 | 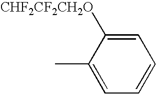 |
| 26 (HCl salt) | —CH₃ | 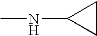 | 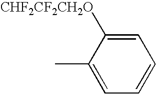 |
| 27 | —CH₃ | 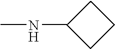 | 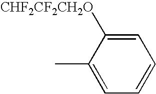 |
| 28 (HCl salt) | —CH₃ | 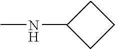 | 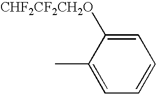 |
| 29 | —CH₃ | 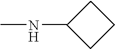 | 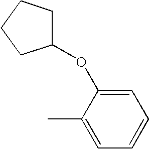 |
| 30 (HCl salt) | —CH₃ | 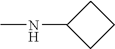 | 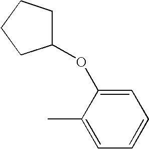 |
| 31 | 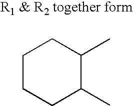 | | 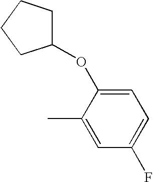 |
| 32 (HCl salt) | 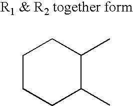 | | 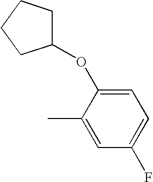 |
| 33 | —CH₃ | —NHCH₃ | 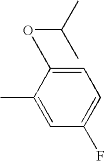 |
| 34 (HCl salt) | —CH₃ | —NHCH₃ | 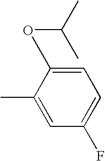 |
| 35 | —CH₃ | —NHCH₃ | 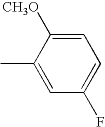 |
| 36 (HCl salt) | —CH₃ | —NHCH₃ | 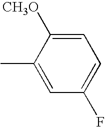 |
| 37 | —CH₃ | —CH₃ | 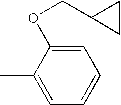 |
| 38 (HCl salt) | —CH₃ | —CH₃ | 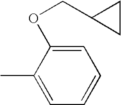 |
| 39 | 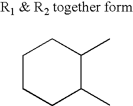 | | 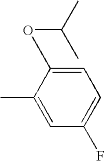 |
| 40 (HCl salt) | 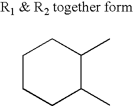 | | 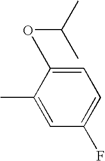 |
| 41 | 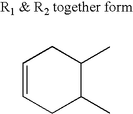 | | 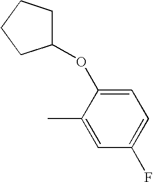 |
| 42 (HCl salt) | 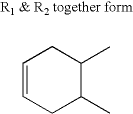 | | 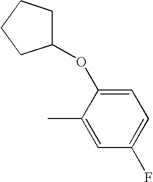 |
| 43 | —CH₃ | 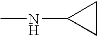 | 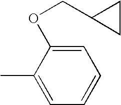 |
| 44 (HCl salt) | —CH₃ | 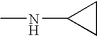 | 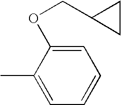 |
| 45 | 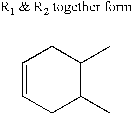 | | 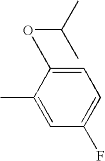 |
| 46 (HCl salt) | 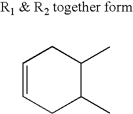 | | 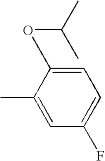 |
| 47 | —CH₃ | 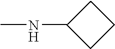 | 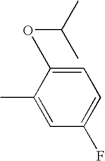 |
| 48 (HCl salt) | —CH₃ | 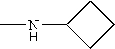 | 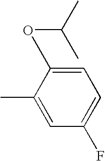 |
| 49 | —CH₃ | —NHCH₃ | 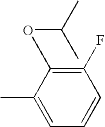 |
| 50 (HCl salt) | —CH₃ | —NHCH₃ | 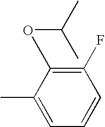 |
| 51 | —CH₃ | 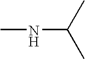 | 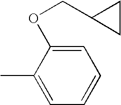 |
| 52 (HCl salt) | —CH₃ | 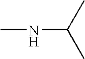 | 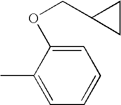 |
| 53 | —CH₃ | —CH₃ | 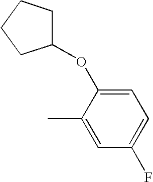 |
| 54 (HCl salt) | —CH₃ | —CH₃ | 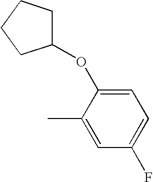 |
| 55 | —CH₃ | 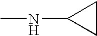 | 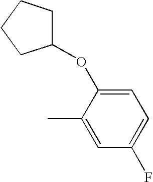 |
| 56 (HCl salt) | —CH₃ | 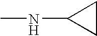 | 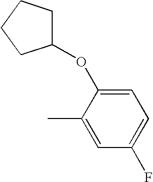 |
| 57 | —CH₃ | 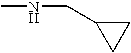 | 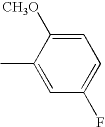 |
| 58 (HCl salt) | —CH₃ | 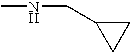 | 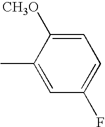 |
| 59 | —CH₃ | 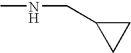 | 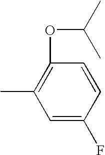 |
| 60 (HCl salt) | —CH₃ | 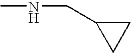 | 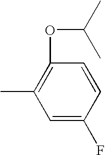 |
| 61 | —CH₃ | 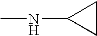 | 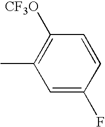 |
| 62 (HCl salt) | —CH₃ | 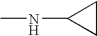 | 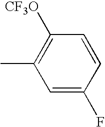 |
| 63 | —CH₃ | 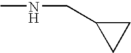 | 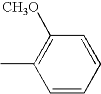 |
| 64 (HCl salt) | —CH₃ | 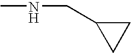 | 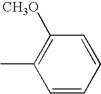 |
| 65 | —CH₃ | 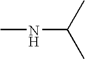 | 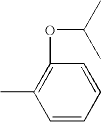 |
| 66 (HCl salt) | —CH₃ | 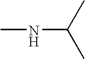 | 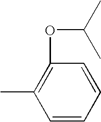 |
| 67 | —CH₃ | 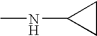 | 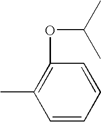 |
| 68 (HCl salt) | —CH₃ | 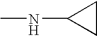 | 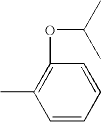 |
| 69 | —CH₃ | 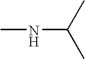 | 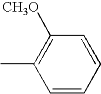 |
| 70 (HCl salt) | —CH₃ | 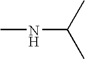 | 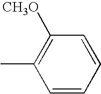 |
| 71** | —CH₃ | —CH₃ | 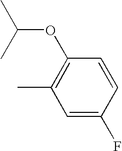 |
| 72** (HCl salt) | —CH₃ | —CH₃ | 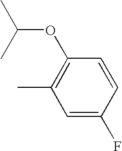 |
| 73 | —CH₃ | —CH₃ | 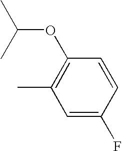 |
| 74 (HCl salt) | —CH₃ | —CH₃ | 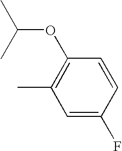 |
| 75 | —CH₃ | 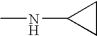 | 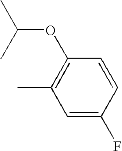 |
| 76 (HCl salt) | —CH₃ | 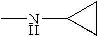 | 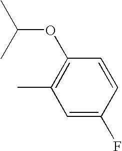 |
| 77 | —CH₃ | 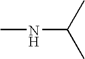 | 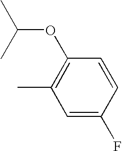 |
| 78 (HCl salt) | —CH₃ | 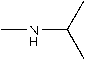 | 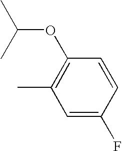 |
| 79 | —CH₃ | 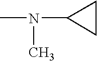 | 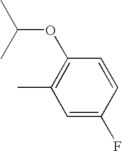 |
| 80 (HCl salt) | —CH₃ | 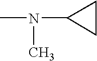 | 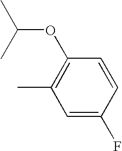 |
| 81** | —CH₃ | —CH₃ | 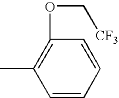 |
| 82** (HCl salt) | —CH₃ | —CH₃ | 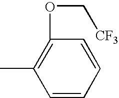 |
| 83 | —CH₃ | —CH₃ | 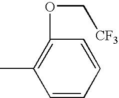 |
| 84 (HCl salt) | —CH₃ | —CH₃ | 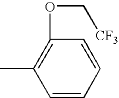 |
| 85** | —CH₃ | —CH₃ | 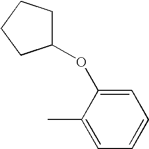 |
| 86** (HCl salt) | —CH₃ | —CH₃ | 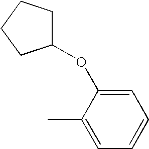 |
| 87 | —CH₃ | —CH₃ | 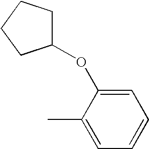 |
| 88 (HCl salt) | —CH₃ | —CH₃ | 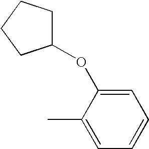 |
| 89 | —CH₃ | 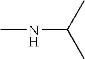 | 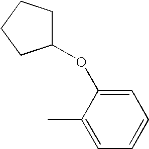 |
| 90 (HCl salt) | —CH₃ | 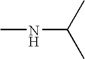 | 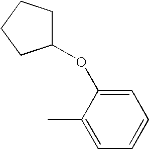 |
| 91 | —CH₃ | 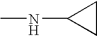 | 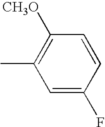 |
| 92 (HCl salt) | —CH₃ | 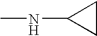 | 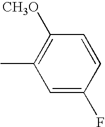 |
| 93 | —CH₃ | —NHCH₃ | 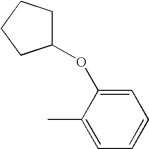 |
| 94 (HCl salt) | —CH₃ | —NHCH₃ | 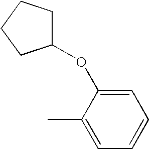 |
| 95 | —CH₃ | —CH₃ | 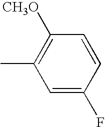 |
| 96 (HCl salt) | —CH₃ | —CH₃ | 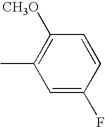 |
| 97** | —CH₃ | —CH₃ | 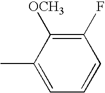 |
| 98** (HCl salt) | —CH₃ | —CH₃ | 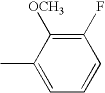 |
| 99 | —CH₃ | —CH₃ | 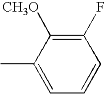 |
| 100 (HCl salt) | —CH₃ | —CH₃ | 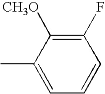 |
| 101 | —CH₃ | —NHCH₃ | 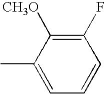 |
| 102 (HCl salt) | —CH₃ | —NHCH₃ | 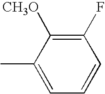 |
| 103 | —CH₃ | 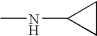 | 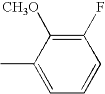 |
| 104 (HCl salt) | —CH₃ | 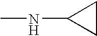 | 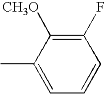 |
| 105 | —CH₃ | —CH₃ | 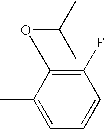 |
| 106 (HCl salt) | —CH₃ | —CH₃ | 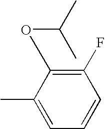 |
| 107 | —CH₃ | 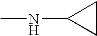 | 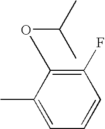 |
| 108 (HCl salt) | —CH₃ | 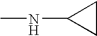 | 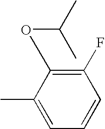 |
| 109 | 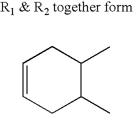 | | 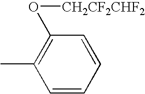 |
| 110 (HCl salt) | 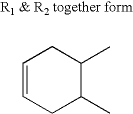 | | 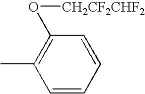 |
| 111* | 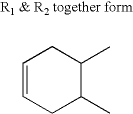 | | 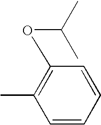 |
| 112* (HCl salt) | 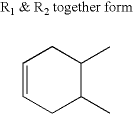 | | 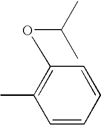 |
| 113 | 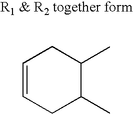 | | 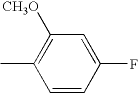 |
| 114 (HCl salt) | 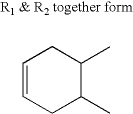 | | 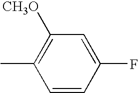 |
| 115 | 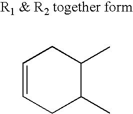 | | 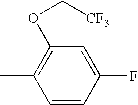 |
| 116 (HCl salt) | 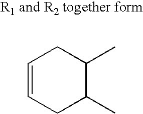 | | 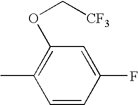 |
| 117 | 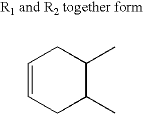 | | 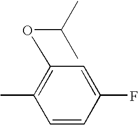 |
| 118 (HCl salt) | 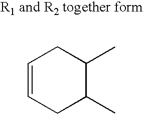 | | 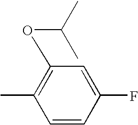 |
| 119 | 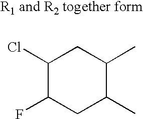 | | 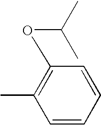 |
| 120 (HCl salt) | 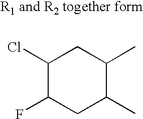 | | 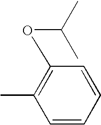 |
| 121 | —CH₃ | H | 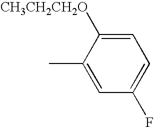 |
| 122 (HCl salt) | —CH₃ | H | 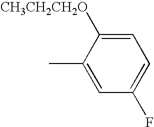 |
| 123 | 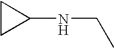 | H | 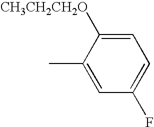 |
| 124 (HCl salt) | 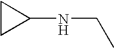 | H | 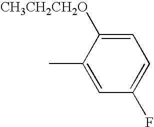 |
| 125 | 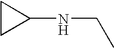 | H | 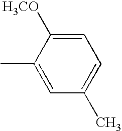 |
| 126 (HCl salt) | 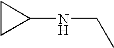 | H | 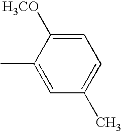 |
| 127 | 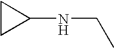 | H | 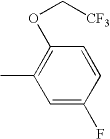 |
| 128 (HCl salt) | 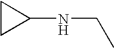 | H | 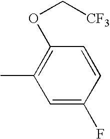 |
| 129 | 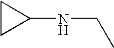 | H | 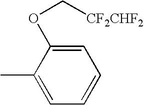 |
| 130 (HCl salt) | 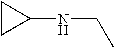 | H | 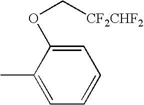 |
| 131 | 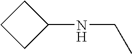 | H | 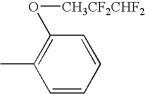 |
| 132 (HCl salt) | 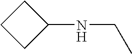 | H | 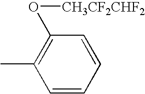 |
| 133 | —CH₃ | H | 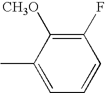 |
| 134 (HCl salt) | —CH₃ | H | 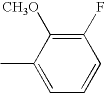 |
| 135 | 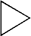 | H | 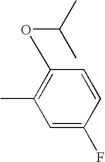 |
| 136 (HCl salt) | 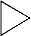 | H | 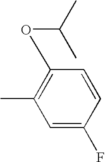 |
| 137 | 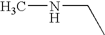 | H | 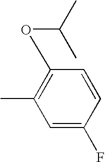 |
| 138 (HCl salt) | 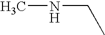 | H | 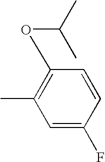 |
| 139 | 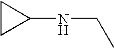 | H | 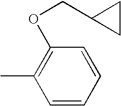 |
| 140 (HCl salt) | 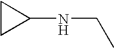 | H | 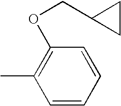 |
| 141 | 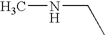 | H | 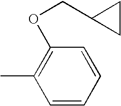 |
| 142 (HCl salt) | 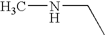 | H | 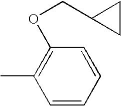 |
| 143 | —CH₃ | H | 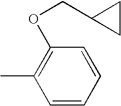 |
| 144 (HCl salt) | —CH₃ | H | 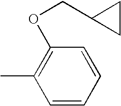 |
| 145 | 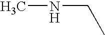 | H | 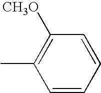 |
| 146 (HCl salt) | 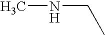 | H | 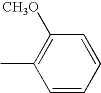 |
| 147 | 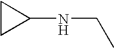 | H | 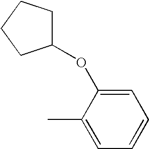 |
| 148 (HCl salt) | 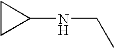 | H | 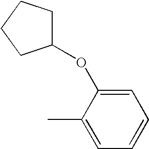 |
| 149 | 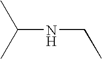 | H | 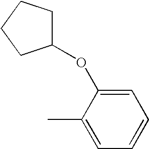 |
| 150 (HCl salt) | 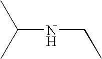 | H | 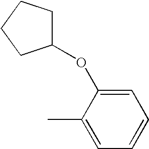 |
| 151 | 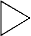 | H | 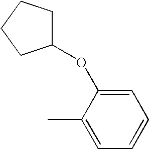 |
| 152 (HCl salt) | 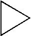 | H | 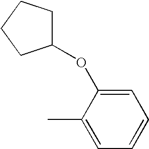 |
| 153 | 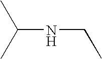 | H | 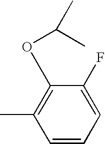 |
| 154 (HCl salt) | 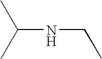 | H | 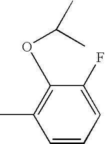 |
| 155 | 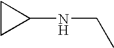 | H | 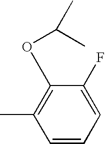 |
| 156 (HCl salt) | 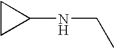 | H | 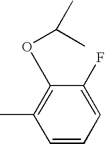 |
| 157 | 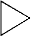 | H | 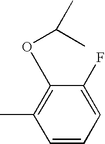 |
| 158 (HCl salt) | 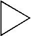 | H | 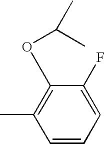 |
| 159 | 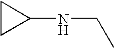 | H | 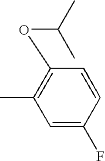 |
| 160 (HCl salt) | 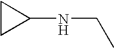 | H | 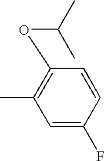 |
| 161 | 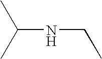 | H | 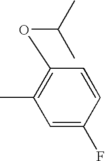 |
| 162 (HCl salt) | 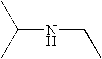 | H | 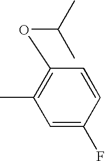 |
| 163 | 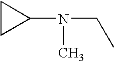 | H | 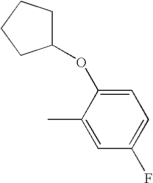 |
| 164 (HCl salt) | 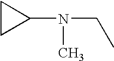 | H | 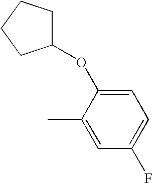 |
| 165 | 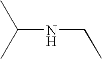 | H | 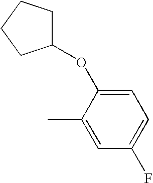 |
| 166 (HCl salt) | 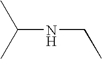 | H | 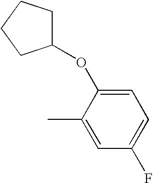 |
| 167 | 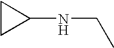 | H | 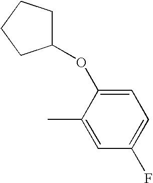 |
| 168 (HCl salt) | 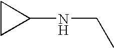 | H | 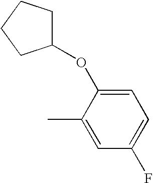 |
| 169 | —CH₃ | H | 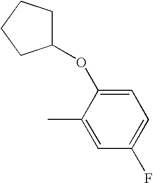 |
| 170 (HCl salt) | —CH₃ | H | 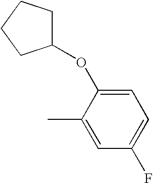 |
| 171** | —CH₃ | H | 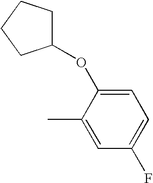 |
| 172** (HCl salt) | —CH₃ | H | 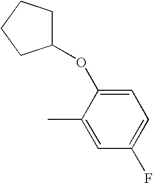 |
| 173 | —CH₃ | 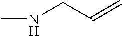 | 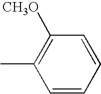 |
| 174 (HCl salt) | —CH₃ | 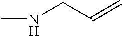 | 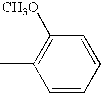 |
| 175 | 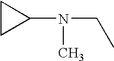 | H | 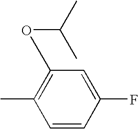 |
| 176 (HCl salt) | 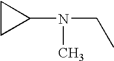 | H | 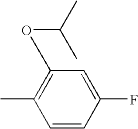 |
| 177 | 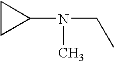 | H | 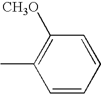 |
| 178 (HCl salt) | 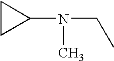 | H | 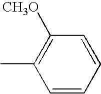 |
| 179 | 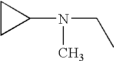 | H | 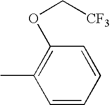 |
| 180 (HCl salt) | 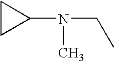 | H | 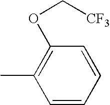 |
| 181 | 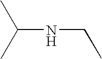 | H | 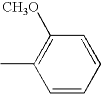 |
| 182 (HCl salt) | 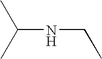 | H | 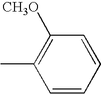 |
| 183 | 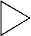 | H | 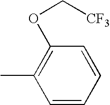 |
| 184 (HCl salt) | 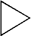 | H | 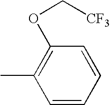 |
| 185** | —CH₃ | H | 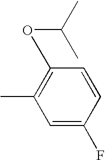 |
| 186** (HCl salt) | —CH₃ | H | 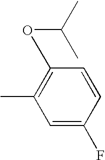 |
| 187 | —CH₃ | H | 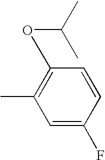 |
| 188 (HCl salt) | —CH₃ | H | 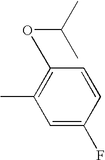 |
| 189** | —CH₃ | H | 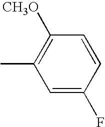 |
| 190** (HCl salt) | —CH₃ | H | 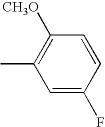 |
| 191 | —CH₃ | H | 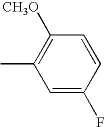 |
| 192 (HCl salt) | —CH₃ | H | 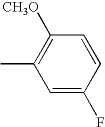 |
| 193 | —CH₃ | H | 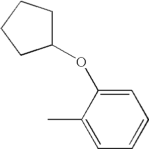 |
| 194 (HCl salt) | —CH₃ | H | 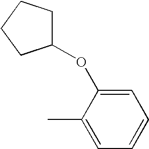 |
| 195** | —CH₃ | H | 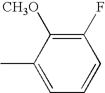 |
| 196** (HCl salt) | —CH₃ | H | 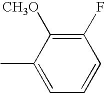 |
| 197 | —CH₃ | H | 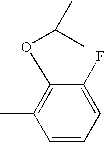 |
| 198 (HCl salt) | —CH₃ | H | 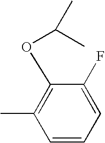 |

*are the compounds where n = 2

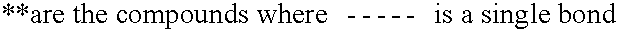

The salts described herein may be prepared by the useful prior art techniques, such as suspending the compound in water and then adding one equivalent of an organic acid such as acetic, oxalic acid, maleic acid, tartaric acid, citric acid, succinic acid, malonic acid, adipic acid, ascorbic acid, camphoenic acid, nicotinic acid, butyric acid, lactic acid, glucuronic acid, or inorganic acids such as hydrochloric acid, hydrobromic acid, phosphoric acid, sulfuric acid, nitric acid, boric acid and perchloric acid.

The neutral solution of the resulting salt is subjected to rotary evaporation under diminished presence to the volume necessary to ensure precipitation of the salt upon cooling, which is then filtered and dried. The salts of the present invention may also be prepared under strictly non-aqueous conditions. For example, dissolving free base in an organic solvent such as ethanol, methanol, isopropanol, dichloromethane or diethyl ether adding exactly one equivalent of the desired acid to the same solvent and stirring the solution at 0° C. to 5° C., causes the precipitation of the acid addition salt, which is then filtered, washed free from the solvent, and dried.

Alternatively, the solvent is stripped completely to obtain the desired salt. These salts are often preferred for use in formulating the therapeutic composition of the invention because they are crystalline and relatively more stable and water soluble. The compounds described herein have got pharmacological activity, therefore may be administered to an animal for treatment orally, topically, rectally, internasally, or by parenteral route. The pharmaceutical compositions of the present invention comprise a pharmaceutically effective amount of a compound of the present invention formulated together with one or more pharmaceutically acceptable carriers. The term “pharmaceutically acceptable carriers” includes non-toxic, inert solid, semi-solid or liquid filter, diluent, encapsulating material or formulation auxiliary of any type. Solid form preparations for oral administration, include capsules, tablets, pills, powders, granules cathets and suppositories. For solid form preparations, the active compound is mixed with at least one inert, pharmaceutically acceptable excipient or carrier such as sodium citrate, dicalcium phosphate and/or a filler or extenders such as starch, lactose, sucrose, glucose, mannitol and silicic acid; binders such as carboxymethylcellulose, alginates, gelatins, polyvinylpyrrolidinone, sucrose, acacia; disintegrating agents such as a agar-agar, calcium carbonate, potato starch, alginic acid, certain silicates and sodium carbonate, absorption accelators such as quaternary ammonium compounds; wetting agents such as cetyl alcohol, glycerol, monostearate; adsorbents such as kaolin; lubricants such as talc, calcium stearate, magnesium stearate, solid polyethyleneglycol, sodium lauryl sulphate and mixture thereof.

In the case of capsules, tablets, or pills, the dosage form may also comprise buffering agents. The solid preparation of tablets, capsules, pills, granules can be prepared with coating and shells such as enteric coating and other coatings well known in the pharmaceutical formulating art.

Liquid form preparations for oral administration include pharmaceutically acceptable emulsions, solution, suspensions, syrups and elixirs. For liquid form preparations, the active compound can be mixed with water or other solvent, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (such as cottonseed, groundnut, corn, germ, olive, castor and Sesamie oil), glycerol, and fatty acid esters of sorbitan and mixture thereof. Besides inert diluents, the oral composition can also include adjuvant such as wetting agents, emulsifying agents, suspending agents, sweetening agents, flavoring agents and perfuming agents.

Injectable preparations such as sterile injections, aqueous or oleaginous suspensions may be formulated according to the art using suitable dispersing or wetting and suspending agents. Among the acceptable vehicles and solvents that may be employed are water, Ringer's solution, U.S.P. and isotonic sodium chloride.

Dosage forms for tropical or transdermal administration of compounds provided herein include ointments, pastes, creams, lotions, gel, powders, solutions, spray, inhalants or patches. The active compound is admixed under sterile condition with a pharmaceutically acceptable carrier and any needed preservative or buffer as may be required. Ophthalmic formulation, eardrops, eye ointments, powder and solution are also provided.

The pharmaceutical preparation may be in unit dosage form. In such form, the preparation may be subdivided into unit doses containing appropriate quantities of the active component. The unit dosage form can be a packaged preparation, the package containing discrete capsules, powders, in vials or ampoules and ointments, capsules, cachet, tablet, gel cream itself or it can be the appropriate number of any of their packaged forms.

The formulation of the present invention may be formulated so as to provide quick, sustained, or delayed release of the active ingredient after administration to the patient by employing procedures well known to the art.

The dosages of the compounds described herein, muscarinic receptor antagonists and 5 α-reductase inhibitors are adjusted when combined to achieve desired effects. As those skilled in the art will appreciate, dosages of the compounds described herein, muscarinic receptor antagonist and 5 α-reductase inhibitor may be independently optimized and combined to achieve a synergistic result wherein the pathology is reduced more than it would be if either agent were used alone. In accordance with methods provided herein, the individual components of combinations can be administered separately at different times during the course of therapy or concurrently in divided or single combination forms.

The examples mentioned below demonstrate general synthetic procedures for the preparation of representative compounds. The examples are provided to illustrate particular aspect of the disclosure and do not limit the scope of the present invention as defined by the claims.

### EXPERIMENTAL DETAILS

### Example 1

### Preparation of 1-{3-[4-(5-Fluoro-2-isopropoxy-phenyl)-piperazin-1-yl-propyl]-3-methyl-pyrrole-2,5-dione hydrochloride salt (Compound No. 186)

### Step 1: Preparation of 3-[4-(5-Fluoro-2-isopropoxy-phenyl)-piperazin-1-yl]-propionitrile

To a solution of 1-(5-Fluoro-2-isopropoxyphenyl)-piperazine (5 gm, 0.021 mol) in methanol (25 ml) was added acrylonitrile (1.34 gm, 0.025 mol) under stirring at room temperature. The reaction mixture was stirred for about 3 to 4 hours. After completion of the reaction, the reaction mass was concentrated on buchi to yield the desired product. Yield: 6 gm, (98%).

### Step 2: Preparation of 3-[4-(5-Fluoro-2-isopropoxy-phenyl)-piperazin-1-yl]-propylamine

To absolution of 3-[4-(5-Fluoro-2-isopropoxy-phenyl)-piperazin-1-yl]-propionitrile (5 gm, 0.017 mol) in methanol-ammonia (20 ml) was added Palladium-Carbon (10% w/w of 3-[4-(5-Fluoro-2-isopropoxy-phenyl)-piperazin-1-yl]-propionitrile) and the reaction mixture was hydrogenated at 55 to 60 psi for about 4 to 5 hours. After completion of the reaction, the reaction mixture was filtered through celite pad, washed with methanol; filtrate thus obtained was concentrated to yield the required compound. Yield: 5 gm, (98%).

### Step 3: Preparation of 1-{3-[4-(5-Fluoro-2-isopropoxy-phenyl)-piperazin-1-yl]-propyl}-3-methyl-pyrrole-2,5-dione

A solution of 3-[4-(5-Fluoro-2-isopropoxy-phenyl)-piperazin-1-yl]-propylamine (1 gm, 0.0034 mol) and citraconic anhydride (0.38 gm, 0.0034 mol) in toluene (15 ml) was refluxed for about 1 hour. The reaction mixture was concentrated to yield the crude product which was purified on the column of silica gel (60-120 mesh) using dichloromethane-methanol mixture as eluent. Yield: 1 gm, (77%).

### Step 4: Preparation of 1-{3-[4-(5-Fluoro-2-isopropoxy-phenyl)-piperazin-1-yl]-propyl)-3-methyl-pyrrole-2,5-dione hydrochloride salt

An equimolar quantity of isopropyl alcohol-hydrochloric acid was added to 1-{3-[4-(5-Fluoro-2-isopropoxy-phenyl)-piperazin-1-yl]-propyl}-3-methyl-pyrrole-2,5-dione and the resulting salt was solidified by adding ether to it.

IR: 1706.5 cm⁻¹; ¹HNMR (300 MHz, CDCl₃)δ: 1.47-1.53 (6H, d), 2.08 (3H, s), 2.25 (2H, s), 3.17 (2H, s), 3.17 (2H, s), 3.64-3.66 (6H, d), 4.06 (2H, s), 4.06-4.69 (5H, m), 6.35 (1H, m), 6.93-7.73 (3H, m); Mass (m/z): 390 (M+1).

The following compounds were prepared similarly.

Compound No. 172: 1-{3-[4-(2-Cyclopentyloxy-5-fluoro-phenyl)-piperazin-1-yl]-propyl}-3-methyl-pyrrole-2,5-dione hydrochloride salt.
Compound No. 190: 1-{3-[4-(5-Fluoro-2-methoxy-phenyl)-piperazin-1-yl]-propyl}-3-methyl-pyrrole-2,5-dione hydrochloride salt.

IR (KBr): 1708.3 cm⁻¹; ¹HNMR (300 MHz, CDCl₃)δ: 2.10 (3H, s), 2.27-2.33 (2H, t), 3.04 (4H, s), 3.51-3.67 (8H, m), 3.83 (3H, s), 6.36 (1H, s), 6.36-6.79 (3H, m); Mass (m/z): 362.3 (M⁺+1).
Compound No. 196: 1-{3-[4-(3-Fluoro-2-methoxy-phenyl)-piperazin-1-yl]-propyl}-3-methyl-pyrrole-2,5-dione hydrochloride salt.

¹HNMR (300 MHz, CDCl₃)δ: 2.11 (3H, s), 2.29 (2H, s), 3.12 (2H, s), 3.59-3.67 (8H, m), 4.09 (3H, s), 4.22 (2H, s), 6.37 (1H, s), 7.00-7.05 (3H, m); Mass (m/z): 362 (M⁺+1).

### Example 2

### Preparation of 1-{3-[(5-Fluoro-2-isopropoxy-phenyl)-piperazin-1-yl]propyl}-3-cyclopropylamino-4-methyl-pyrrolidine-2,5-dione hydrochloride salt (Compound No. 76)

### Step 1: Preparation of 1-{3-[4-(5-Fluoro-2-isopropoxy-phenyl)-piperazin-1-yl]-propyl}-3-cyclopropylamino-4-methyl-pyrrolidine-2,5-dione

To a solution of 1-{3-[4-(5-Fluoro-2-isopropoxy-phenyl)-piperazin-1-yl]-propyl}-3-methyl-pyrrole-2,5-dione (0.5 gm, 0.0013 mol) in methanol was added equimolar quantity of cyclopropylamine (0.073 gm, 0.0013 mol) and the reaction mixture was stirred at room temperature for about 10 to 12 hours. The reaction mixture was concentrated to yield the crude product which was then purified on a column of silica gel (60-120 mesh) using dichloromethane-methanol mixture as eluent. Yield: 0.256 gm, (45%).

### Step 2: Preparation of 1-{3-[4-(5-Fluoro-2-isopropoxy-phenyl)-piperazin-1-yl]-propyl}-3-cyclopropylamino-4-methyl-pyrrolidine-2,5-dione hydrochloride salt

An equimolar quantity of isopropyl alcohol-hydrochloric acid was added to
1-{3-[4-(5-Fluoro-2-isopropoxy-phenyl)-piperazin-1-yl]-propyl}-3-cyclopropylamino 4-methyl-pyrrolidine-2,5-dione and the resulting salt was solidified by adding ether to it.

¹HNMR (300 MHz, CDCl₃)δ: 0.59 (4H, s), 1.11-1.45 (6H, m), 1.53 (3H, s), 2.15 (1H, s), 2.27 (1H, s), 2.61-2.67 (1H, d), 3.17-3.29 (6H, t), 3.48 (5H, s), 3.69 (3H, s), 4.45-4.53 (1H, m), 6.62-6.80 (3H, m); Mass (m/z): 447 (M⁺+1).

The following compounds were prepared similarly.

Compound No. 2: 1-{3-[4-(5-Fluoro-2-propoxy-phenyl)-piperazin-1-yl]-propyl}-3-methyl-4-methylamino-pyrrolidine-2,5-dione hydrochloride salt.

¹HNMR (300 MHz, CDCl₃)δ: 1.02-1.07 (3H, t), 1.35-1.38 (3H, t), 1.30-1.87 (2H, q), 2.23-2.27 (2H, t), 2.34-2.39 (1H, d), 2.95-3.03 (6H, m), 3.51 (5H, s), 3.61-3.69 (2H, m), 3.90-3.94 (2H, m), 6.62-6.79 (3H, m); Mass (m/z): 392 (M⁺+1).
Compound No. 6: 1-{3-[4-(5-Fluoro-2-propoxy-phenyl)-piperazin-1-yl]-propyl}-3-cyclopropylamino-4-methyl-pyrrolidine-2,5-dione hydrochloride salt.

¹HNMR (300 MHz, CDCl₃)δ: 0.658-0.881 (5H, m), 1.008-1.057 (3H, t), 1.785-1.854 (3H, m), 2.289 (4H, s), 2.655-2.715 (2H, d), 3.250-3.507 (13H, m), 3.888-3.931 (2H, t), 6.625-6.789 (3H, m); Mass (m/z): 447 (M⁺+1).
Compound No. 12: 1-{3-[4-(2-Methoxy-5-methyl-phenyl)-piperazin-1-yl]-propyl}-3-methyl-4-methylamino-pyrrolidine-2,5-dione hydrochloride salt.

IR (KBr): 1714.9 cm⁻¹; Mass: 389 (M⁺1).
Compound No. 14: 1-{3-[4-(2-Methoxy-1-methyl-phenyl)-piperazin-1-yl]-propyl}-3-cyclopropylamino-4-methyl-pyrrolidine-2,5-dione hydrochloride salt.

IR (KBr): 1700.2 cm⁻¹; Mass (m/z): 415 (M⁺+1).
Compound No. 16: 1-{3-[4-(5-Fluoro-2-propoxy-phenyl)-piperazin-1-yl]-propyl}-3-cyclobutylamino-4-methyl-pyrrolidine-2,5-dione hydrochloride salt.

IR (KBr): 1713.1 cm⁻¹; Mass (m/z): 461.36 (M+1).
Compound No. 18: 1-(3-{4-[5-Fluoro-2-(2,2,2-trifluoro-ethoxy)-phenyl]-piperazin-1-yl}-propyl)-3-cyclopropylamino-4-methyl-pyrrolidine-2,5-dione hydrochloride salt.

IR (KBr): 1716 cm⁻¹; Mass (m/z): 487 (M⁺+1).
Compound No. 20: 1-(3-{4-[5-Fluoro-2-(2,2,2-fluoro-ethoxy)-phenyl]-piperazin-1-yl}-propyl)-3-methyl-4-methylamino-pyrrolidine-2,5-dione hydrochloride salt.

IR (KBr): 1716.6 cm⁻¹; Mass (m/z): 461.25 (M⁺+1).
Compound No. 26: 1-(3-{4-[2-(2,2,3,3-Tetrafluoro-propoxy)-phenyl]-piperazin-1-yl}-propyl)-3-cyclopropylamino-4-methyl-pyrrolidine-2,5-dione hydrochloride salt.

IR (KBr): 1704.1 cm⁻¹; Mass (m/z): 501 (M+1).
Compound No. 28: 1-(3-{4-[2-(2,2,3,3-Tetrafluoro-propoxy)-phenyl]-piperazin-1-yl}-propyl)-3-cyclobutylamino-4-methyl-pyrrolidine-2,5-dione hydrochloride salt.

IR (DCM): 1713.8 cm⁻¹; Mass (m/z): 515 (M⁺+1).
Compound No. 30: 1-{3-[4-(2-Cyclopentyloxy-phenyl)-piperazin-1-yl]-propyl}-3-cyclobutylamino-4-methyl-pyrrolidine-2,5-dione hydrochloride salt.

IR (KBr): 1704.5 cm⁻¹; Mass (m/z): 469-(M⁺+1).
Compound No. 34: 1-{3-[4-(5-Fluoro-2-isopropoxy-phenyl)-piperazin-1-yl]-propyl}-3-methyl-4-methylamino-pyrrolidine-2,5-dione hydrochloride salt.

IR (KBr): 1714 cm⁻¹; Mass (m/z): 421 (M⁺+1).
Compound No. 36: 1-{3-[4-(5-Fluoro-2-methoxy-phenyl)-piperazin-1-yl]-propyl}-3-methyl-4-methylamino-pyrrolidine-2,5-dione hydrochloride salt.

IR (KBr): 1695.3 cm⁻¹; Mass (m/z): 393 (M⁺+1).
Compound No. 44: 1-{3-[4-(2-Cyclopropylmethoxy-phenyl)-piperazin-1-yl]-propyl}-3-cyclopropylamino-4-methyl-pyrrolidine-2,5-dione hydrochloride salt.

¹HNMR (300 MHz, DMSO)δ: 0.33-0.35 (2H, d), 0.57-0.59 (2H, d), 0.69 (5H, s), 1.10 (1H, s), 1.24-1.25 (2H, d), 1.57 (3H, s), 2.60-2.65 (2H, d), 2.87-3.56 (15H, m), 6.93-7.01 (4H, m); Mass (m/z): 441 (M+1).
Compound No. 48: 1-{3-[4-(5-Fluoro-2-isopropoxy-phenyl)-piperazin-1-yl]-propyl}-3-cyclobutylamino-4-methyl-pyrrolidine-2,5-dione hydrochloride salt.

IR (KBr): 1707 cm⁻¹; Mass (m/z): 4610M+1).
Compound No. 50: 1-{3-[4-(3-Fluoro-2-isopropoxy-phenyl)-piperazin-1-yl]-propyl}-3-methyl-4-methylamino-pyrrolidine-2,5-dione hydrochloride salt.

IR (KBr): 1703 cm⁻¹; Mass (m/z): 421 (M⁺+1).
Compound No. 52: 1-{3-[4-(2-Cyclopropylmethoxy-phenyl)-piperazin-1-yl]-propyl}-3-isopropylamino-4-methyl-pyrrolidine-2,5-dione hydrochloride salt.

¹HNMR (300 MHz, DMSO)δ: 0.33-0.58 (5H, m), 1.24-1.37 (11H, m), 1.65 (2H, s), 2.05-2.07 (2H, d), 2.95-3.17 (8H, m), 3.83-3.86 (3H, d), 6.88-6.97 (4H, m); Mass (m/z): 443.52 (M⁺+1).
Compound No. 56: 1-{3-[4-(2-Cyclopentyloxy-5-fluoro-phenyl)-piperazin-1-yl]-propyl}-3-cyclopropylamino-4-methyl-pyrrolidine-2,5-dione hydrochloride salt.
Compound No. 58: 1-{3-[4-(5-Fluoro-2-methoxy-phenyl)-piperazin-1-yl]-propyl}-3-(cyclopropylmethyl-amino)-4-methyl-pyrrolidine-2,5-dione hydrochloride salt.

IR (KBr): 1704.2 cm⁻¹; Mass (m/z): 433 (M⁺+1).
Compound No. 60: 1-{3-[4-(5-Fluoro-2-isopropoxy-phenyl)-piperazin-1-yl]-propyl}-3-(cyclopropylmethyl-amino)-4-methyl-pyrrolidine-2,5-dione hydrochloride salt.

IR (KBr): 1688.7 cm⁻¹; Mass (m/z): 461 (M⁺+1).
Compound No. 62: 1-{3-[4-(5-Fluoro-2-trifluoromethoxy-phenyl)-piperazin-1-yl]-propyl}-3-cyclopropylamino-4-methyl-pyrrolidine-2,5-dione hydrochloride salt.

IR (KBr): 1704.1 cm⁻¹; Mass (m/z): 455 (M⁺+1).
Compound No. 64: 1-{3-[4-(2-Methoxy-phenyl)-piperazin-1-yl]-propyl}-3-(cyclopropylmethyl-amino)-4-methyl-pyrrolidine-2,5-dione hydrochloride salt.

IR (KBr): 1695.1 cm⁻¹; Mass (m/z): 415 (M⁺+1).
Compound No. 66: 1-{3-[4-(2-Isopropoxy-phenyl)-piperazin-1-yl]-propyl}-3-isopropylamino-4-methyl-pyrrolidine-2,5-dione hydrochloride salt.

IR: 1707.4 cm⁻¹; Mass (m/z): 430 (M⁺+1).
Compound No. 68: 1-{3-[4-(2-Isopropoxy-phenyl)-piperazin-1-yl]-propyl}-3-cyclopropylamino-4-methyl-pyrrolidine-2,5-dione hydrochloride salt.

IR: 1695.6 cm⁻¹; Mass: 428 (M⁺+1).
Compound No. 70: 1-{3-[4-(2-Methoxy-phenyl)-piperazin-1-yl]-propyl}-3-isopropylamino-4-methyl-pyrrolidine-2,5-dione hydrochloride salt.

IR: 1715.2 cm⁻¹; Mass: 402 (M+1).
Compound No. 78: 1-{3-[4-(5-Fluoro-2-isopropoxy-phenyl)-piperazin-1-yl]-propyl}-3-isopropylamino-4-methyl-pyrrolidine-2,5-dione hydrochloride salt.

¹HNMR (300 MHz, CDCl₃)δ: 1.209-1.368 (6H, m), 1.415-1.547 (3H, t), 1.638-1.655 (3H, d), 1.748 (4H, s), 2.401 (2H, s), 2.721-2.779 (1H, d), 2.779-3.343 (4H, brs), 3.446-3.991 (10H, m), 4.411-4.469 (1H, q), 6.567-6.782 (3H, m); Mass (m/z): 449 (M+1).
Compound No. 80: 1-{3-[4-(5-Fluoro-2-isopropoxy-phenyl)-piperazin-1-yl]-propyl}-3-(cyclopropyl-methyl-amino)-4-methyl-pyrrolidine-2,5-dione hydrochloride salt.

IR (KBr): 1715.1 cm⁻¹; H (300 MHz, DMSO)δ: 0.831-0.853 (4H, d), 1.257-1.276 (6H, d), 1.691 (3H, s), 2.018 (2H, s), 2.775 (3H, s), 2.893-2.954 (3H, d), 3.163 (4H, s), 3.467-3.637 (8H, q), 4.007 (1H, s), 6.769-6.799 (3H, m); Mass (m/z): 461 (M⁺+1).
Compound No. 90: 1-{3-[4-(2-Cyclopentyloxy-phenyl)-piperazin-1-yl]-propyl}-3-isopropylamino-4-methyl-pyrrolidine-2,5-dione hydrochloride salt.

IR (KBr): 1714 cm⁻¹; Mass (m/z): 457 (M⁺+1).
Compound No. 92: 1-{3-[4-(5-Fluoro-2-methoxy-phenyl)-piperazin-1-yl]-propyl}-3-cyclopropylamino-4-methyl-pyrrolidine-2,5-dione hydrochloride salt.

R (KBr): 1693.6 cm⁻¹; ¹HNMR (300 MHz, CDCl₃)δ: 1.481 (3H, s), 2.037 (1H, s), 2.244 (2H, s), 2.575-2.635 (1H, d), 3.053 (2H, s), 3.170-3.231 (2H, d), 3.462 (6H, s), 3.656-3.699 (2H, t), 3.835 (3H, s), 6.645-6.789 (3H, m); Mass (m/z): 419.2 (M⁺+1).
Compound No. 94: 1-{3-[4-(2-Cyclopentyloxy-phenyl)-piperazin-1-yl]-propyl}-3-methyl-4-methylamino-pyrrolidine-2,5-dione hydrochloride salt.

IR (KBr): 1716.3 cm⁻¹; ¹HNMR (300 MHz, CDCl₃)δ: 1.62-2.03 (1H, m), 2.36 (3H, s), 2.76-2.80 (4H, d), 3.20 (4H, s), 3.35-3.77 (9H, m), 4.76 (1H, s), 6.81-6.98 (4H, m); Mass (m/z): 492 (M+1).
Compound No. 102: 1-{3-[4-(3-Fluoro-2-methoxy-phenyl)-piperazin-1-yl]-propyl}-3-methyl-methylamino-pyrrolidine-2,5-dione hydrochloride salt.

IR (KBr): 1721 cm⁻¹; ¹HNMR (300 MHz, DMSO)δ: 1.556 (3H, s), 1.981 (2H, s), 2.549 (3H, s), 2.844-3.553 (1SH, m), 3.840 (3H, s), 6.799-7.079 (3H, m); Mass (m/z): 393 (M⁺+1).
Compound No. 104: 1-{3-[4-(3-Fluoro-2-methoxy-phenyl)-piperazin-1-yl]-propyl}-3-cyclopropylamino-4-methyl-pyrrolidine-2,5-dione hydrochloride salt.

IR (KBr): 1695.8 cm⁻¹; ¹HNMR (300 MHz, CDCl₃)δ: 0.540-0.595 (5H, t), 1.501 (3H, s), 2.071 (1H, s), 2.250-2.271 (2H, d), 2.586-2.647 (1H, d), 3.102-3.708 (13H, m), 3.906 (3H, s), 6.670-6.960 (3H, m); Mass (m/z): 419 (M+1).
Compound No. 108: 1-{3-[4-(3-Fluoro-2-isopropoxy-phenyl)-piperazin-1-yl]-propyl}-3-cyclopropylamino-4-methyl-pyrrolidine-2,5-dione hydrochloride salt.

¹HNMR (300 MHz, CDCl₃)δ: 0.528-0.556 (4H, d), 1.184-1.362 (9H, m), 2.062 (1H, s), 2.238 (2H, s), 2.582-2.643 (1H, d), 3.067 (4H, s), 3.187-3.248 (3H, d), 3.361 (5H, s), 3.513 (2H, s), 4.472-4.512 (1H, t), 6.667-6.983 (3H, m); Mass (m/z): 447 (M⁺+1).
Compound No. 174: 1-{3-[4-(2-Methoxy-phenyl)-piperazin-1-yl]-propyl}-3-methyl-4-prop-2-ynylamino-pyrrolidine-2,5-dione hydrochloride salt.

IR: 1701.5 cm⁻¹; Mass: 398 (M⁺+1).

### Example 3

### Preparation of 1-{3-[4-(5-Fluoro-2-isopropoxy-phenyl-piperazin-1-yl]-propyl}-3,4-dimethyl-pyrrole-2,5-dione hydrochloride salt (Compound No. 72)

### Step 1: Preparation of 1-{3-[4-(5-Fluoro-2-isopropoxy-phenyl)-piperazin-1-yl]-propyl}-3,4-dimethyl-pyrrole-2,5-dione

A solution of 3-[4-(5-Fluoro-2-isopropoxy-phenyl)-piperazin-1-yl]-propylamine (1 gm, 0.0034 mol) and 3,4-dimethylfuran-2,5-dione (0.43 gm, 0.0034 mol) in toluene (15 ml) was refluxed for about 1 hour. The reaction mixture was concentrated to yield the crude product which was then purified on the column of silica gel (60-120 mesh) using dichloromethane-methanol mixture as eluent. Yield: 0.8 gm, (59%).

### Step 2: Preparation of 1-{3-[4-(5-Fluoro-2-isopropoxy-phenyl)piperazin-1-yl]-propyl}-3,4-dimethyl-pyrrole-2,5-dione hydrochloride salt

An equimolar quantity of isopropyl alcohol-hydrochloric acid was added to 1-{3-[4-(5-Fluoro-2-isopropoxy-phenyl)-piperazin-1-yl]-propyl}-3,4-dimethyl-pyrrole-2,5-dione and the resulting salt was solidified by adding ether to it.

IR: 1691 cm⁻¹; ¹HNMR (300 MHz, CDCl₃)δ: 1.25-1.33 (6H, t), 1.979 (6H, s), 2.282 (2H, s), 3.008-3.027 (4H, d), 3.536-3.660 (8H, m), 4.457-4.517 (1H, m), 6.613-6.806 (3H, m); Mass (m/z): 404 (M⁺+1).

The following compounds were prepared similarly.

Compound No. 82: 1-(3-{4-[2-(2,2,2-Trifluoro-ethoxy)-phenyl]-piperazin-1-yl}-propyl)-3, 4-dimethyl-pyrrole-2,5-dione hydrochloride salt.

IR (KBr): 1647 cm⁻¹; Mass (m/z): 425 (M⁺+1).
Compound No. 86: 1-{3-[4-(2-Cyclopentyloxy-phenyl)-piperazin-1-yl]-propyl}-3,4-dimethyl-pyrrole-2,5-dione hydrochloride salt.

IR (KBr): 1702.4 cm⁻¹; Mass (m/z): 412 (M⁺+1).
Compound No. 98: 1-{3-[4-(3-Fluoro-2-methoxy-phenyl)-piperazin-1-yl]-propyl}-3,4-dimethyl-pyrrole-2,5-dione hydrochloride salt.

¹HNMR (300 MHz, CDCl₃)δ: 1.992 (6H, s), 2.206 (2H, s), 3.155-3.207 (2H, t), 3.479-3.517 (2H, d), 3.632-3.671 (6H, t), 3.818-3.859 (2H, d), 4.039-4.071 (3H, d), 6.990-7.075 (4H, m); Mass (m/z): 376 (M⁺+1).

### Example 4

### Preparation of 2-{3-[4-(2-Cyclopentyloxy-5-fluoro-phenyl)-piperazin-1-yl]-propyl}-3a,4,7,7a-tetrahydro-isoindole-1,3-dione hydrochloride salt (Compound No. 42)

### Step 1:Preparation of 2-(3-Chloropropyl)-3a,4,7,7a-tetrahydro-isoindole-1,3-dione

### Step 2: Preparation of 2-{3-[4-(2-Cyclopentyloxy-5-fluoro-phenyl)-piperazin-1-yl]-propyl}-3a,4,7,7a-tetrahydro-isoindole-1,3-dione

A suspension of 2-(3-Chloropropyl)-3a,4,7,7a-tetrahydro-isoindole-1,3-dione (1 gm, 0.0044 mol), 1-(2-Cyclopentyloxy-5-fluoro-phenyl)-piperazine (0.9 gm, 0.0037 mol), anhydrous potassium carbonate (1.2 gm, 0.0087 mol) and potassium iodide (0.014 gm, 0.00008 mol) was heated in dimethylformamide (15 ml) at 70-75° C. for about 6-8 hours. Reaction was quenched by adding water (45 ml) to it; extracted with ethyl acetate. The organic layers were combined and dried over anhydrous sodium sulphate and concentrated to yield the crude product. It was then purified on a column of silica gel (60-120 mesh) using dichloromethane—methanol mixture as eluent. Yield: 1.5 gm, (75%).

### Step 3: 2-{3-[4-(2-Cyclopentyloxy-5-fluoro-phenyl)-piperazin-1-yl]-propyl}-3a,4,7,7a-tetrahydro-isoindole-1,3-dione hydrochloride salt

An equimolar quantity of isopropyl alcohol-hydrochloric acid was added to 2-{3-[4-(2-Cyclopentyloxy-5-fluoro-phenyl)-piperazin-1-yl]-propyl}-3a,4,7,7a-tetrahydro-isoindole-1,3-dione and the resulting salt was solidified by adding ether to it.

IR (KBr): 1700.2 cm⁻¹; ¹HNMR (300 MHz, CDCl₃)δ: 1.70-1.74 (8H, m), 1.85-1.91 (4H, d), 2.21-2.25 (2H, d), 2.95 (4H, s), 3.16-3.17 (2H, d), 3.51-3.64 (8H, m), 4.75 (1H, s), 5.92-5.94 (2H, t), 6.61-6.76 (3H, m); Mass (m/z): 456 (M⁺+1).

The following compounds were prepared similarly

Compound No. 46: 2-{3-[4-(5-Fluoro-2-isopropoxy-phenyl)-piperazin-1-yl]-propyl}-3a,4,7,7a-tetrahydro-isoindole-1,3-dione hydrochloride salt.

IR (KBr): 1692.8 cm⁻¹; ¹HNMR (300 MHz, CDCl₃)δ: 1.32-1.34 (6H, d), 2.21-2.26 (4H, q), 2.61-2.66 (2H, d), 2.94-3.00 (4H, m), 3.16 (2H, s), 3.47-3.64 (8H, m), 4.48-4.50 (1H, d), 5.92-5.53 (2H, d), 6.61-6.78 (3H, m); Mass (m/z): 430 (M⁺+1).
Compound No. 110: 2-(3-{4-[2-(2,2,3,3-Tetrafluoro-propoxy)-phenyl]-piperazin-1-yl}-propyl)-3a,4,7,7a-tetrahydro-isoindole-1,3-dione hydrochloride salt.

IR: 1698.8 cm⁻¹; ¹HNMR (300 MHz, CDCl₃)δ: 2.21-2.25 (4H, m), 2.62-2.67 (2H, d), 3.00-3.17 (6H, m), 3.46-3.74 (8H, m), 4.39-4.48 (2H, m), 5.94-6.11 (3H, m), 6.90-7.13 (4H, m), 13.00 (1H, brs); Mass (m/z): 484.1 (M⁺+1).
Compound No. 112: 2-{4-[4-[2-Isopropoxy-phenyl]-piperazin-1-yl}-butyl}-3a,4,7,7a-tetrahydro-isoindole-1,3-dione hydrochloride salt.

IR KBr): 1699.4 cm⁻¹; ¹HNMR (300 MHz, CDCl₃)δ: 1.35-1.37 (6H, d), 1.68 (2H, m), 1.91 (2H, m), 2.20-2.25 (2H, d), 2.59-2.64 (2H, d), 3.03 (4H, m), 3.17 (2H, m), 3.53 (8H, m), 4.57-4.61 (1H, m), 5.91 (2H, brs), 6.85-7.03 (4H, m), 12.58 (1H, brs); Mass (m/z): 425.9 (M+1).
Compound No. 114: 2-{3-[4-(4-Fluoro-2-methoxy-phenyl)-piperazin-1-yl]-propyl}-3a,4,7,7a-tetrahydro-isoindole-1,3-dione hydrochloride salt.

IR (KBr): 1695.7 cm⁻¹; ¹HNMR (300 MHz, CDCl₃)δ: 2.21-2.25 (4H, m), 2.61-2.66 (2H, dd), 2.98 (4H, brs), 3.17 (2H, brs), 3.39-3.76 (8H, m), 3.85 (3H, s), 5.93 (2H, brs), 6.61-6.64 (3H, d), 6.88 (1H, m), 12.75 (1H, brs); Mass (m/z): 402 (M⁺+1).
Compound No. 116: 2-(3-{4-[4-Fluoro-2-(2,2,2-trifluoro-ethoxy)-phenyl]-piperazin-1-yl}-propyl)-3a,4,7,7a-tetrahydro-isoindole-1,3-dione hydrochloride salt.

IR (KBr): 1697.3 cm⁻¹; ¹HNMR (300 MHz, DMSO-d₆)δ: 1.91 (2H, m), 2.18-2.22 (2H, m), 2.38-2.43 (2H, m), 2.96-3.35 (14H, m), 4.75 (2H, m), 5.89 (2H, brs), 6.83-6.88 (1H, m), 7.00-7.07 (2H, m), 10.28 (1H, brs); Mass (m/z): 470.0 (M+1).
Compound No. 118: 2-{3-[4-(4-Fluoro-2-isopropoxy-phenyl)-piperazin-1-yl]-propyl}-3a,4,7,7a-tetrahydro-isoindole-1,3-dione hydrochloride salt.

IR (KBr): 1696.5 cm⁻¹; ¹HNMR (300 MHz, DMSO-d₆)δ: 1.27-1.29 (6H, d), 1.88-1.90 (2H, m), 2.18-2.23 (2H, m), 2.37-2.43 (2H, m), 2.89-3.17 (12H, m), 4.63-4.67 (1H, m), 5.86-5.89 (2H, d), 6.66-6.72 (1H, m), 6.88-6.94 (2H, m), 10.18 (1H, brs); Mass (m/z): 418.0 (M⁺+1).

### Example 5

### Preparation of 1-{3-[4-(5-Fluoro-2-isopropoxy-phenyl)-piperazin-1-yl]-propyl]-3,4-dimethyl-pyrrolidine-2,5-dione hydrochloride salt (Compound No. 74)

### Step 1: Preparation of 1-{3-[4-(5-Fluoro-2-isopropoxy-phenyl)-piperazin-1-yl]-propyl}-3,4-dimethyl-pyrrolidine-2,5-dione

A mixture of 1-{3-[4-(5-Fluoro-2-isopropoxy-phenyl)-piperazin-1-yl]-propyl}-3,4-dimethyl-pyrrole-2,5-dione (0.5 gm, 0.0012 mol) and palladium-carbon (0.5 gm) in methanol was hydrogenated at 45 to 50 psi for about 1 hour. The reaction mixture was concentrated to yield the desired product. Yield: 0.5 gm, (99%).

### Step 2: Preparation of 1-{3-[4-(5-Fluoro-2-isopropoxy-phenyl)-piperazin-1-yl]-propyl}-3,4-dimethyl-pyrrolidine-2,5-dione hydrochloride salt

An equimolar quantity of isopropyl alcohol-hydrochloric acid was added to 1-{3-[4-(5-Fluoro-2-isopropoxy-phenyl)-piperazin-1-yl]-propyl}-3,4-dimethyl-pyrrolidine-2,5-dione and the resulting salt was solidified by adding ether to it.

¹HNMR (300 MHz, CDCl₃)δ: 1.18-1.44 (12H, m), 2.26 (2H, s), 3.04-3.11 (6H, d), 3.44-3.63 (8H, m), 4.47-4.55 (1H, m), 6.67-6.82 (3H, m); Mass (m/z): 406.2 (M⁺+1).

The following compounds were prepared similarly.

Compound No. 4: 2-(3-{4-[2-(2,2,3,3-Tetrafluoro-propoxy)-phenyl]-piperazin-1-yl}-propyl)-hexahydro-isoindole-1,3-dione hydrochloride salt.

IR (KBr): 1716.3 cm⁻¹; Mass (m/z): 486 (M⁺+1).
Compound No. 10:1-{3-[4-(2-Methoxy-5-methyl-phenyl)-piperazin-1-yl]-propyl}-3,4-dimethyl-pyrrolidine-2,5-dione hydrochloride salt.

IR (KBr): 1703.4 cm⁻¹; Mass: 374 (M⁺+1).
Compound No. 22: 1-(3-{4-[5-Fluoro-2-(2,2,2-trifluoro-ethoxy)-phenyl]-piperazin-1-yl}-propyl)-3,4-dimethyl-pyrrolidine-2,5-dione hydrochloride salt.

IR (KBr): 1695.1 cm⁻¹; Mass (m/z): 446 (M⁺+1).
Compound No. 24: 1-(3-{4-[2-(2,2,3,3-Tetrafluoro-propoxy)-phenyl]-piperazin-1-yl}-propyl)-3,4-dimethyl-pyrrolidine-2,5-dione hydrochloride salt.

IR (KBr): 1699 cm⁻¹; Mass (m/z): 460 (M⁺+1).
Compound No. 32: 2-{3-[4-(2-Cyclopentyloxy-5-fluoro-phenyl)-piperazin-1-yl]-propyl}-hexahydro-isoindole-1,3-dione hydrochloride salt.

¹HNMR (300 MHz, CDCl₃)δ: 1.462 (5H, s), 1.891 (8H, s), 2.229-2.252 (3H, d), 2.942 (3H, s), 3.061 (2H, s), 3.509-3.560 (2H, d), 3.611-3.653 (9H, m), 4.800 (1H, s), 6.776-6.803 (3H, m); Mass (m/z): 458 (M⁺+1).
Compound No. 38: 1-{3-[4-(2-Cyclopropylmethoxy-phenyl)-piperazin 1-yl]-propyl}-3,4-dimethyl-pyrrolidine-2,5-dione hydrochloride salt.

¹HNMR (300 MHz, CDCl₃)δ: 0.33-0.35 (2H, d), 0.62-0.63 (2H, d), 1.23-1.25 (7H, d), 2.01 (2H, s), 3.02-3.03 (6H, d), 3.55-3.86 (10H, m), 6.81-7.03 (4H, m).
Compound No. 40: 2-{3-[4-(5-Fluoro-2-isopropoxy-phenyl)-piperazin-1-yl]-propyl}-hexahydro-isoindole-1,3-dione hydrochloride salt.

IR (KBr): 1692.4 cm⁻¹; ¹HNMR (300 MHz, CDCl₃)δ: 1.306-1.326 (6H, d), 1.438-1.456 (4H, d), 1.508 (1H, s), 1.760 (2H, s), 1.846 (2H, s), 2.006-2.034 (2H, d), 2.796 (2H, s), 2.898 (4H, s), 3.317 (4H, s), 3.578-3.624 (3H, m), 4.479-4.519 (1H, t), 6.604-6.768 (3H, m); Mass (m/z): 432 (M⁺+1).
Compound No. 54: 1-{3-[4-(2-Cyclopentyloxy-5-fluoro-phenyl)-piperazin-1-yl]-propyl}-3,4-dimethyl-pyrrolidine-2,5-dione hydrochloride salt.

IR (KBr): 1700.3 cm⁻¹; ¹HNMR (300 MHz, CDCl₃)δ: 1.355-1.379 (3H, d), 1.669-1.913 (11H, m), 2.266 (2H, s), 2.961-3.061 (6H, m), 3.516-3.654 (8H, m), 4.746 (1H, s), 6.613-6.789 (3H, m); Mass (m/z): 432 (M⁺+1).
Compound No. 84: 1-(3-{4-[2-(2,2,2-Trifluoro-ethoxy)-phenyl]-piperazin-1-yl}-propyl)-3, 4-dimethyl-pyrrolidine-2,5-dione hydrochloride salt.

IR (KBr): 1704 cm⁻¹; Mass (m/z): 428 (M⁺+1).
Compound No. 88: 1-{3-[4-(2-Cyclopentyloxy-phenyl)-piperazin-1-yl]-propyl}-3,4-dimethyl-pyrrolidine-2,5-dione hydrochloride salt.

IR (KBr): 1697 cm⁻¹; Mass (m/z): 413 (M+1).
Compound No. 96: 1-{3-[4-(5-Fluoro-2-methoxy-phenyl)-piperazin-1-yl]-propyl}-3,4-dimethyl-pyrrolidine-2,5-dione hydrochloride salt.

¹HNMR (300 MHz, CDCl₃)δ: 1.224-1.246 (6H, d), 2.230-2.282 (2H, t), 3.031-3.048 (6H, d), 3.498-3.648 (8H, m), 3.837 (3H, s), 6.659-6.781 (3H, m); Mass (m/z): 378 (M⁺+1).
Compound No. 100: 1-{3-[4-(3-Fluoro-2-methoxy-phenyl)-piperazin-1-yl]-propyl}-3,4-dimethyl-pyrrolidine-2,5-dione hydrochloride salt.

IR (KBr): 1693.0 cm⁻¹; ¹HNMR (300 MHz, CDCl₃)δ: 1.227-1.246 (6H, d), 2.239-2.288 (2H, t), 3.039-3.115 (6H, m), 3.555-3.651 (8H, m), 3.911 (3H, s), 6.685-6.993 (3H, m); Mass (m/z): 378 (M++).
Compound No. 106: 1-{3-[4-(3-Fluoro-2-isopropoxy-phenyl)-piperazin-1-yl]-propyl}-3,4-dimethyl-pyrrolidine-2,5-dione hydrochloride salt.

¹HNMR (300 MHz, CDCl₃)δ: 1.24-1.35 (12H, t), 2.27 (2H, s), 3.05-3.21 (6H, t), 3.44-3.64 (8H, m), 4.61 (1H, s), 6.86-6.96 (3H, m); Mass (m/z): 406 (M⁺+1).
Compound No. 122: 1-{3-[4-(5-Fluoro-2-propoxy-phenyl)-piperazin-1-yl]-propyl}-3-methyl-pyrrolidine-2,5-dione hydrochloride salt.

¹H NMR (300 MHz, CDCl₃)δ: 1.016-1.065 (3H, t), 1.569 (3H, s), 1.790-1.859 (2H, q), 2.142 (2H, s), 2.439 (3H, s), 2.605-2.667 (2H, d), 2.984-3.048 (4H, d), 3.363 (4H, s), 3.649-3.667 (2H, t), 3.888-3.931 (2H, t), 6.606-6.752 (3H, m); Mass (m/z): 421 (M⁺+1).
Compound No. 134: 1-{3-[4-(3-Fluoro-2-methoxy-phenyl)-piperazin-1-yl]-propyl}-3-methyl-pyrrolidine-2,5-dione hydrochloride salt.

IR (KBr): 1702.9 cm⁻¹; ¹HNMR (300 MHz, CDCl₃)δ: 1.356-1.379 (3H, t), 2.258-2.392 (3H, m), 2.414-3.078 (6H, m), 3.479-3.658 (8H, m), 3.912 (3H, s), 6.680-6.994 (3H, m); Mass (m/z): 364 (M⁺+1).
Compound No. 144: 1-{3-[4-(2-Cyclopropylmethoxy-phenyl)-piperazin-1-yl]-propyl}-3-methyl-pyrrolidine-2,5-dione hydrochloride salt.

¹HNMR (CDCl₃, 300 MHz)δ: 0.33-0.34 (2H, d), 0.61-0.62 (2H, d), 0.65 (1H, s), 1.35-1.37 (3H, d), 2.21-2.38 (3H, m), 2.94-3.03 (6H, m), 3.47-3.51 (6H, d), 3.60-3.65 (2H, t), 3.83-3.86 (2H, d), 6.80-7.00 (4H, m).
Compound No. 170: 1-{3-[4-(2-Cyclopentyloxy-5-fluoro-phenyl)-piperazin-1-yl]-propyl}-3-methyl-pyrrolidine-2,5-dione hydrochloride salt.
Compound No. 188: 1-{3-[4-(5-Fluoro-2-isopropoxy-phenyl)-piperazin-1-yl]-propyl}-3-methyl-pyrrolidine-2,5-dione hydrochloride salt.

IR: 1699.6 cm⁻¹; ¹HNMR (300 MHz, CDCl₃)δ: 1.32-1.34 (6H, d), 2.26-2.39 (3H, t), 2.96-3.12 (6H, t), 3.53-3.63 (8H, d), 4.47-4.51 (1H, t), 6.63-6.80 (3H, m); Mass (m/z): 392 (M⁺+1).
Compound No. 192: 1-{3-[4-(5-Fluoro-2-methoxy-phenyl)-piperazin-1-yl]-propyl}-3-methyl-pyrrolidine-2,5-dione hydrochloride salt.

IR: 1694.7 cm⁻¹; ¹HNMR (300 MHz, CDCl₃)δ: 1.354-1.377 (3H, d), 2.235-2.388 (3H, m), 2.944-3.083 (6H, m), 3.502-3.654 (8H, m), 3.837 (3H, s), 6.650-6.810 (3H, m); Mass (m/z): 364.3 (M⁺+1).
Compound No. 194: 1-{3-[4-(2-Cyclopentyloxy-phenyl)-piperazin-1-yl]-propyl}-3-methyl-pyrrolidine-2,5-dione hydrochloride salt.

IR: 1694.2 cm⁻¹; ¹HNMR (300 MHz, CDCl₃)δ: 1.358-1.379 (3H, d), 1.489 (2H, s), 1.668-1.688 (2H, d), 1.880-2.053 (4H, m), 2.249-2.392 (3H, t), 2.962-3.035 (2H, t), 3.146 (2H, s), 3.574-3.652 (6H, t), 3.947-3.991 (2H, d), 4.316 (2H, s), 4.881 (1H, s), 6.939-7.296 (4H, m); Mass (m/z): 400 (M⁺+1).
Compound No. 198: 1-{3-[4-(3-Fluoro-2-isopropoxy-phenyl)-piperazin-1-yl]-propyl}-3-methyl-pyrrolidine-2,5-dione hydrochloride salt.

¹HNMR (300 MHz, CDCl₃)δ: 1.18-1.30 (6H, m), 1.35-1.37 (3H, d), 2.18-2.23 (2H, t), 2.34-2.38 (1H, d), 2.90-3.03 (5H, m), 3.19 (2H, s), 3.45-3.51 (5H, t), 3.61-3.65 (2H, t), 4.48-4.52 (1H, m), 6.67-6.98 (3H, m).

### Example 6

### Preparation of 2-{3-[4-(2-Cyclopentyloxy-5-fluoro-phenyl)-piperazin-1-yl]-propyl}-5,6-difluoro-hexahydro-isoindole-1,3-dione hydrochloride salt (Compound No. 8)

### Step 1: Preparation of 4-(3-bromopropyl) tetrahydro-1aH-oxireno[f]isoindole-3,5(2H,4-dione

To a solution of 2-(3-bromopropyl)-3a,4,7,7a-tetrahydroisoindole-1,3-dione (1.0 gm, 0.0037 mole) in dichloromethane (10 mL) was added equimolar quantity of m-chloroperbenzoic acid (1.33 gm of 50%, 0.0037 mole) in dichloromethane at 0-5° C. The reaction mixture was stirred for about 6-8 hours. Reaction mixture was poured into ice cold potassium carbonate solution (5%) and concentrated to yield of the desired product.

Yield: 0.8 gm (75%)

### Step 2: Preparation of 4-{3-[4-(2-Cyclopentyloxy-1-fluoro-phenyl)-piperazin-1-yl]-propyl}-hexahydro-1-oxa-4-aza-cyclopropa[f]indene-3,5-dione

A suspension of 4-(3-bromopropyl)hexahydro-1-oxa-4-aza-cyclopropa[f]indene-3,5-dione (0.8 gm, 0.0028 mol), 1-(5-Fluoro-2-cyclopentyloxyphenyl)piperazine (0.7 gm, 0.0028 mol), anhydrous potassium carbonate (0.46 gm, 0.003 mol) and potassium iodide (0.009 gm, 0.00005 mol) in dimethylformamide (20 ml) was heated at 50-55° C. for about 24 hours. Reaction was quenched by adding water (60 ml) to it; extracted with ethyl acetate, the combined organic layer was then dried over anhydrous sodium sulfate and concentrated to yield the crude product. It was then purified on a column of silica gel (60-120 mesh) using dichloromethane and methanol mixture as eluent. Yield: 0.6 gm, (62%).

### Step 3: Preparation of 2-{3-[4-(2-Cyclopentyloxy-5-fluoro-phenyl)-piperazin-1-yl]-propyl}-5,6-difluoro-hexahydro-isoindole-1,3-dione

To the solution of 4-{3-[4-(2-Cyclopentyloxy-5-fluoro-phenyl)-piperazin-1-yl]-propyl}-hexahydro-1-oxa-4-aza-cyclopropa[f]indene-3,5-dione (0.5 gm, 0.001 mol) in dichloromethane (15 ml) was added diethylaminosulfurtifluoride (0.26 gm, 0.0016 mol) dropwise under stirring at 0-5° C. The reaction mixture was allowed to come at room temperature and stirred for about 2-3 hours. After the completion of the reaction, it was quenched by adding dilute solution of sodium bicarbonate; extracted with dichloromethane and combined organic layers were concentrated to yield the crude product. It was then purified on a column of silica gel (60-120 mesh) using dichloromethane and methanol mixture as eluent to yield the desired product. Yield: 0.080 gm, (15%).

### Step 4: Preparation of 2-{3-[4-(2-Cyclopentyloxy-5-fluoro-phenyl)-piperazin-1-yl]-propyl}-5,6-difluoro-hexahydro-isoindole-1,3-dione hydrochloride salt

An equimolar quantity of isopropyl alcohol-hydrochloric acid was added to 2-{3-[4-(2-Cyclopentyloxy-5-fluoro-phenyl)-piperazin-1-yl]-propyl}-5,6-difluoro-hexahydro-isoindole-1, 3-dione and the resulting salt was solidified by adding ether to it. IR: 1704.6 cm⁻¹; Mass (m/z): 494 (M⁺+1); M.P: 189-195° C.

The following compounds are prepared similarly
Compound No. 120: 2-{3-[4-(2-Isopropoxy-phenyl)-piperazine-1-yl]-propyl}-5-chloro-6-fluoro-hexahydro-isoindole-1,3-dione hydrochloride salt.

IR: 1701.5 cm⁻¹; ¹HNMR (300 MHz, DMSO-d₆)δ: 1.27-1.29 (611, d), 1.97-2.34 (6H, m), 2.98-3.24 (10H, m), 4.47 (1H, m), 4.56-4.66 (1H, m), 4.82-4.98 (1H, m), 6.88-6.96 (4H, m), 10.53 (1H, brs); Mass (m/z): 466.3 (M⁺+1).

### Example 7

### Preparation of 1-{3-[4-(5-Fluoro-2-isopropoxy-henyl-piperazin-1-yl-propyl}-3-cyclopropylamino-methyl-pyrrolidine-2,5-dione hydrochloride salt (Compound No. 160)

### Step 1: Preparation of 1-{3-[4-(5-Fluoro-2-isopropoxy-phenyl)-piperazin-1-yl]-propyl}-3-methylene-pyrrolidine-2,5-dione

A solution of 3-[4-(5-Fluoro-2-isopropoxy-phenyl)-piperazin-1-yl]-propylamine (1.0 gm, 0.0034 mol) and itaconic anhydride (0.38 gm, 0.0034 mol) in toluene (15 ml) was refluxed for about 1 hour. After completion of the reaction the reaction mixture was concentrated to form a crude residue, which was then purified by column chromatography.

Yield: 0.7 gm, (54%)

### Step 2: Preparation of 1-{3-[4-(5-Fluoro-2-isopropoxy-phenyl)-piperazin-1-yl]-propyl}-3-cyclopropylamino-methyl-pyrrolidine-2,5-dione

To a solution of 1-{3-[4-(5-Fluoro-2-isopropoxy-phenyl)-piperazin-1-yl]-propyl}-3-methylene-pyrrolidine-2,5-dione (0.5 gm, 0.0013 mol) in methanol was added equimolar quantity of cyclopropylamine (0.073 gm, 0.0013 mol) and reaction mixture was stirred at room temperature for about 10-12 hours. The reaction mixture was concentrated to yield the crude product which was then purified on a column of silica gel (60-120 mesh) using dichloromethane and methanol mixture as eluent to yield the desired product. Yield: 0.3 gm, (53%).

### Step 3: Preparation of 1-{3-[4-(5-Fluoro-2-isopropoxy-phenyl)-piperazin-1-yl-propyl}-3-cyclopropylamino-methyl-pyrrolidine-2,5-dione hydrochloride salt

An equimolar quantity of isopropyl alcohol-hydrochloric acid was added to 1-{3-[4-(5-Fluoro-2-isopropoxy-phenyl)-piperazin-1-yl]-propyl}-3-cyclopropylamino-methyl-pyrrolidine-2, 5-dione and the resulting salt was solidified by adding ether to it IR (KBr): 1698.8 cm⁻¹; ¹HNMR (CDCl₃, 300 MHz)δ: 0.74 (2H, s), 0.92 (2H, s), 1.20-1.23 (1H, m), 1.29-1.31 (61, d), 1.65 (3H, s), 2.33-2.40 (3H, d), 2.69-2.75 (1H, d), 3.15 (2H, s), 3.45-3.51 (12H, m), 4.44-4.48 (1H, t), 6.61-6.79 (3H, m); Mass (m/z): 447 (M⁺+1).

The following compounds are prepared similarly
Compound No. 124: 1-{3-[4-(5-Fluoro-2-propoxy-phenyl)-piperazin-1-yl]-propyl}-3-cyclopropylamino-methyl-pyrrolidine-2,5-dione hydrochloride salt.

IR (KBr): 1605 cm⁻¹; Mass (m/z): 447 (M⁺+1).
Compound No. 126: 1-{3-[4-(2-methoxy-5-methyl-phenyl)-piperazin-1-yl]-propyl}-3-cyclopropylaminomethyl-pyrrolidine-2,5-dione hydrochloride salt.

IR (KBr): 1704.4 cm⁻¹; Mass (m/z): 415 (M⁺+1).
Compound No. 128: 1-(3-{4-[5-Fluoro-2-(2,2,2-trifluoro-ethoxy)-phenyl]-piperazin-1-yl}-propyl)-3-cyclopropylaminomethyl-pyrrolidine-2,5-dione hydrochloride salt.

IR (KBr): 1716.8 cm⁻¹; Mass (m/z): 487 (M+1).
Compound No. 130: 1-(3-{4-[2-(2,2,3,3-Tetrafluoro-propoxy)-phenyl]-piperazin-1-yl}-propyl)-3-cyclopropylaminomethyl-pyrrolidine-2,5-dione hydrochloride salt.

IR (KBr): 1702.1 cm⁻¹; Mass (m/z): 501 (M⁺+1).
Compound No. 132: 1-(3-{4-[2-(2,2,3,3-Tetrafluoro-propoxy)-phenyl]-piperazin-1-yl}-propyl)-3-cyclobutylaminomethyl-pyrrolidine-2,5-dione hydrochloride salt.

IR (DCM): 1709.2 cm⁻¹; Mass (m/z): 515 (M+1).
Compound No. 138: 1-{3-[4-(5-Fluoro-2-isopropoxy-phenyl)-piperazin-1-yl]-propyl}-3-methylaminomethyl-pyrrolidine-2,5-dione hydrochloride salt.

IR (KBr): 1693 cm⁻¹; Mass (m/z): 421 (M⁺+1).
Compound No. 140: 1-{3-[4-(2-Cyclopropylmethoxy-phenyl)-piperazin-1-yl]-propyl}-3-cyclopropylamino-methyl-pyrrolidine-2,5-dione hydrochloride salt.

IR (KBr): 1702.6 cm⁻¹; ¹HNMR (300 MHz, CDCl₃)δ: 0.317-0.332 (2H, d), 0.610-0.636 (4H, d), 0.846-0.883 (2H, s), 1.256 (2H, s), 2.328 (4H, s), 2.692-2.752 (2H, d), 3.126-3.723 (14H, m), 3.822-3.845 (2H, d), 6.803-7.017 (4H, m); Mass (m/z): 441 (M⁺+1).
Compound No. 142: 1-{3-[4-(2-Cyclopropylmethoxy-phenyl)-piperazin-1-yl]-propyl}-3-methylamino-methyl-pyrrolidine-2,5-dione hydrochloride salt.

¹HNMR (300 MHz, DMSO)δ: 0.33-0.34 (2H, d), 0.55-0.58 (3H, d), 1.24-1.26 (3H, d), 2.07 (2H, s), 2.61 (3H, s), 2.84-2.90 (3H, d), 2.99 (3H, s), 3.50-3.55 (9H, t), 3.83-3.86 (2H, d), 6.88-6.97 (4H, m).
Compound No. 146: 1-{3-[4-(2-Methoxy-phenyl)-piperazin-1-yl]-propyl}-3-methylaminomethyl-pyrrolidine-2,5-dione hydrochloride salt.

¹HNMR (300 MHz, CDCl₃)δ: 1.841 (3H, s), 2.008 (1H, s), 2.296 (3H, s), 2.464-2.579 (3H, t), 2.669-2.824 (6H, m), 3.111 (4H, s), 3.582-3.627 (2H, t), 3.858 (3H, s), 6.844-6.999 (4H, m); Mass (m/z): 375 (M⁺+1).
Compound No. 148: 1-{3-[4-(2-Cyclopentyloxy-phenyl)-piperazin-1-yl]-propyl}-3-cyclopropylaminomethyl-pyrrolidine-2,5-dione hydrochloride salt.

IR (KBr): 1715.3 cm⁻¹; ¹HNMR (300 MHz, CDCl₃)δ: 0.81 (2H, s), 1.09 (1H, s), 1.20-1.25 (2H, t), 1.62-1.85 (10H, m), 2.34 (2H, s), 2.59 (1H, s), 2.75-2.80 (1H, d), 3.13 (2H, s), 3.39-3.75 (12H, m), 4.77 (1H, s), 6.82-6.98 (3H, m); Mass (m/z): 455 (M⁺+1).
Compound No. 150: 1-{3-[4-(2-Cyclopentyloxy-phenyl)-piperazin-1-yl]-propyl}-3-(isopropylamino-methyl)-pyrrolidine-2,5-dione hydrochloride salt.

IR (KBr): 1705.9 cm⁻¹; ¹HNMR (300 MHz, CDCl₃)δ: 1.50-1.84 (14H, m), 2.32-2.41 (3H, t), 2.72-2.81 (1H, d), 3.20-3.36 (4H, t), 3.44-3.55 (1H, q), 3.79-3.89 (6H, d), 4.75 (1H, s), 6.81-6.98 (3H, m); Mass (m/z): 457 (M⁺+1).
Compound No. 154: 1-{3-[4-(3-Fluoro-2-isopropoxy-phenyl)-piperazin-1-yl]-propyl}-3-(isopropylamino-methyl)-pyrrolidine-2,5-dione hydrochloride salt.

IR (KBr): 1715.3 cm⁻¹; ¹HNMR (300 MHz, CDCl₃)δ: 1.11-2.00 (15H, m), 2.48 (2H, s), 2.75 (1H, s), 3.35-3.93 (1H, m), 4.46 (1H, s), 6.64-6.91 (3H, m); Mass (m/z): 449 (M⁺+1).
Compound No. 156: 1-{3-[4-(3-Fluoro-2-isopropoxy-phenyl)-piperazin-1-yl]-propyl}-3-cyclopropylamino-methyl-pyrrolidine-2,5-dione hydrochloride salt.

IR (KBr): 1709.2 cm⁻¹; ¹HNMR (300 MHz, CDCl₃)δ: 0.86-0.88 (4H, d), 1.18-1.41 (6H, m), 1.77 (2H, s), 2.37 (2H, s), 2.58 (1H, s), 2.73-2.79 (1H, d), 3.16-4.07 (14H, m), 4.45 (1H, s), 6.65-6.97 (3H, m); Mass (m/z): 447 (M⁺+1).
Compound No. 162: 1-{3-[4-(5-Fluoro-2-isopropoxy-phenyl)-piperazin-1-yl]-propyl}-3-(isopropylamino-methyl)-pyrrolidine-2,5-dione hydrochloride salt.

IR (KBr): 1698.7 cm⁻¹; ¹HNMR (300 MHz, CDCl₃)δ: 1.199-1.693 (15H, m), 2.006 (21H, s), 2.452 (1H, s), 3.331-4.025 (14H, m), 4.440 (1H, s), 6.606-6.778 (3H, m); Mass (m/z): 449 (M⁺+1).
Compound No. 164: 1-{3-[4-(2-Cyclopentyloxy-5-fluoro-phenyl)-piperazin-1-yl]-propyl}-3-[(cyclopropylmethylamino)methyl]-pyrrolidine-2,5-dione hydrochloride salt.

IR (KBr): 1712.5 cm⁻¹; ¹HNMR (300 MHz, CDCl₃)δ: 1.25 (3H, s), 1.33-1.83 (13H, m), 2.42 (2H, s), 2.82 (2H, s), 3.17 (4H, s), 3.42-3.78 (10H, m), 4.73 (1H, s), 6.60-6.73 (3H, m); Mass (m/z): 487 (M+1).
Compound No. 166: 1-{3-[4-(2-Cyclopentyloxy-5-fluoro-phenyl)-piperazin-1-yl]-propyl}-3-isopropylamino-methyl)-pyrrolidine-2,5-dione hydrochloride salt.
Compound No. 168: 1-{3-[4-(2-Cyclopentyloxy-5-fluoro-phenyl)-piperazin-1-yl]-propyl}-3-cyclopropylaminomethyl-pyrrolidine-2,5-dione hydrochloride salt.
Compound No. 176: 1-{3-[4-(4-Fluoro-2-isopropoxy-phenyl)-piperazin-1-yl]-propyl}-3-[(cyclopropyl-methyl-amino)-methyl]-pyrrolidine-2,5-dione hydrochloride salt.

IR: 1699.9 cm⁻¹; Mass: 460 (M+1).
Compound No. 178: 1-{3-[4-(2-Methoxy-phenyl)-piperazin-1-yl]-propyl}-3-cyclopropyl-methyl-amino-methyl]-pyrrolidine-2,5-dione hydrochloride salt.

IR: 1716.5 cm⁻¹; Mass: 414 (M⁺+1).
Compound No. 180: 1-(3-{4-(2-(2,2,2-Trifluoro-ethoxy)-phenyl]-piperazin-1-yl}-propyl)-3-cyclopropyl-methyl-amino-methyl]-pyrrolidine-2,5-dione hydrochloride salt.

IR: 1707.0 cm⁻¹; Mass: 482 (M+1).
Compound No. 182: 1-{3-[4-(2-Methoxy-phenyl)-piperazin-1-yl]-propyl}-3-(isopropylamino)-methyl)-pyrrolidine-2,5-dione hydrochloride salt.

IR: 1664.9 cm⁻¹; Mass: 402 (M⁺+1).

### Example 8

### Preparation of 5-{3-[4-(5-Fluoro-2-isopropoxy-phenyl)-piperazin-1-yl]-propyl}-5-aza-spiro[2.4]heptane-4,6-dione hydrochloride salt (Compound No. 136)

To a suspension of sodium hydride (0.037 gm, 0.0015 mol) in dimethylsulfoxide (15 ml) was added trimethylsulphoxonium iodide (0.34 gm, 0.0015 mol) in lots at room temperature. It was followed by the addition of solution of 1-{3-[4-(5-Fluoro-2-isopropoxy-phenyl)-piperazin-1-yl]-propyl}-3-methylene-pyrrolidine-2,5-dione (0.5 gm, 0.0013 mol) in dimethylsulfoxide (5 ml) to a clear reaction mixture at 10-15° C. Reaction mixture was stirred for about 10-15 minutes. Reaction was quenched by adding water (30 ml) to it. It was extracted with ethyl acetate; combined organic layers were concentrated to yield the crude product. It was then purified by column chromatography.

Yield: 0.2 gm, (39%)

IR (KBr): 1737 cm⁻¹; Mass (m/z): 404 (M⁺+1).

The following compounds are prepared similarly

Compound No. 152: 5-{3-[4-(2-Cyclopentyloxy-phenyl)-piperazin-1-yl]-propyl}-5-aza-spiro[2,4]heptane-4,6-dione hydrochloride salt.

¹HNMR (300 MHz, CDCl₃)δ: 1.25 (2H, s), 1.39-1.44 (2H, m), 1.52 (2H, s), 1.65 (3H, s), 1.91-2.03 (4H, q), 2.16 (2H, s), 2.30-2.32 (1H, d), 3.11 (2H, s), 3.51-3.61 (6H, d), 4.28 (2H, s), 4.60 (2H, s), 4.91 (1H, s), 6.99-7.38 (3H, m); Mass (m/z): 412.5 (M+1).
Compound No. 158: 5-{3-[4-(3-Fluoro-2-isopropoxy-phenyl)-piperazin-1-yl]-propyl}-5-aza-spiro[2.4]heptane-4,6-dione hydrochloride salt.

¹HNMR (300 MHz, CDCl₃)δ: 1.28-1.30 (6H, d), 1.37-1.51 (4H, m), 2.20-2.31 (4H, t), 2.93-3.02 (4H, d), 3.49-3.59 (8H, d), 4.44-4.50 (1H, m), 6.67-6.99 (3H, m); Mass (m/z): 404 (M⁺+1).
Compound No. 184: 5-(3-{4-[2-(2,2,2-Trifluoro-ethoxy)-phenyl]-piperazin-1-yl}-propyl)-5-aza-spiro[2.4]heptane-4,6-dione hydrochloride salt.

IR: 1703.8 cm⁻¹; Mass: 425 M+1).

Pharmacological Testing

Receptor Binding Assay

Receptor binding assays were performed using native α-1 adrenoceptors. The affinity of different compounds for α₁ₐ and α_{1b} adrenoceptor subtypes was evaluated by studying their ability to displace specific [³H]prazosin binding from the membranes of rat submaxillary and liver respectively (Mchel et al., Br. J. Pharmacol., 9.8, 883-889 (1989)). The binding assays were performed according to U'Prichard et al. (Eur. J. Pharmacol., 50:87-89 (1978) with minor modifications.

Submaxillary glands were isolated immediately after sacrifice. The liver was perfused with buffer (Tris HCl 50 mM, NaCl 100 mM, 10 mM EDTA pH 7.4). The tissues were homogenized in 10 volumes of buffer (Tris HCl 50 mM, NaCl 100 mM, EDTA 10 mM, pH 7.4). The homogenate was filtered through two layers of wet guaze and filtrate was centrifuged at 500 g for 10 min. The supernatant was subsequently centrifuged at 40,000 g for 45 min. The pellet thus obtained was re-suspended in the same volume of assay buffer (Tris HCl 50 mM, EDTA 5 mM, pH 7.4) and were stored at −70° C. until the time of assay.

The membrane homogenates (150-250 μg protein) were incubated in 250 μl of assay buffer (Tris HCl 50 mM, EDTA 5 mM, pH 7.4) at 24-25° C. for 1 hour. Non-specific binding was determined in the presence of 300 nM prazosin. The incubation was terminated by vacuum filtration over GF/B fiber filters. The filters were then washed with ice cold 50 mM Tris HCl buffer (pH 7.4). The filter mats were dried and bounded radioactivity retained on filters was counted. The IC₅₀ and Kd were estimated by using the non-linear curve-fitting program using G pad prism software. The value of inhibition constant Ki was calculated from competitive binding studies by using Cheng and Prusoff equation (Cheng and Prusoff, Biochem. Pharmacol., 1973, 22:3099-3108), Kᵢ=IC₅₀/(1+L/K_{d}) where L is the concentration of [³H] prazosin used in the particular experiment. The pKi values were in the range of about 6.80 to about 11 and about 5 to about 7.5 for α₁ₐ and α_{1b} subtype adrenergic receptors, respectively.

In Vitro Functional Studies

In Vitro alpha-1 Adrenoceptor Selectivity

In order to study selectivity of action of the present compounds towards different alpha-1 adrenoceptor subtypes, the ability of these compounds to antagonize alpha-1-adrenoceptor agonist induced contractile response of aorta (alpha-id), prostate (alpha-1a) and spleen (alpha-1b) was studied. Aorta, prostate and spleen tissue were isolated from thipentane anaesthetized (≈300 mg/Kg) male wistar rats. Isolated tissues were mounted in organ bath containing Krebs Henseleit buffer of the following composition (mM): NaCl 118; KCl 4.7; CaCl₂ 2.5; MgSO₄. 7H₂O 1.2; NaHCO₃ 25; KH₂PO₄ 1.2; glucose 11.1. Buffer was maintained at 37° C. and aerated with a mixture of 95% O₂ and 5% CO₂. A resting tension of 2 g (aorta and spleen) or 1 g (prostate) was applied to tissues. Contractile response was monitored using a force displacement transducer and recorded on chart recorders. Tissues were allowed to equilibrate for 1 and ½ hours. At the end of equilibration period, concentration response curves to norepinephrine (aorta) and phenylephirine (spleen and prostate) were obtained in the absence and presence of the tested compound (at concentration of 0.1, 1 and 10 μM. The pK_{B} values were in the range of 8 to 10, 6.80 to 9 and 7.5 to 9 for α₁ₐ, α_{1b} and α_{1d} subtype adrenergic receptor, respectively.

We claim:

1. A compound having the structure of Formula I, 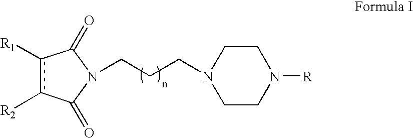
its pharmaceutically acceptable acid addition salts, solvates, enantiomers, diastereomers, regioisomers, N-oxides, polymorphs, prodrugs and metabolites wherein,
— represents no bond or a single bond;
The variable n represents the integers 1 or 2;
R₁ and R₂ are selected from alkyl, cycloalkyl, or 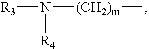
wherein m is the integer 0 or 1;
R₃ is selected from alkyl, or cycloalkyl;
R₄ is selected from hydrogen or alkyl;
R₂ may also be hydrogen; or
R₁ and R₂ can together form a group selected from cycloalkyl or cycloalkenyl; and
R is 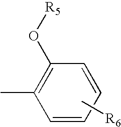
wherein R₅ is selected from alkyl or cycloalkyl, and wherein R₆ is selected from hydrogen, halogen or alkyl.

2. A compound according to claim 1 wherein R₁ is alkyl.

3. A compound according to claim 2 wherein R₁ is methyl.

4. A compound according to claim 1 wherein R₁ is cycloalkyl.

5. A compound according to claim 4 wherein R₁ is cyclopropyl.

6. A compound according to claim 1 wherein R₁ and R₂ together form optionally substituted cycloalkyl wherein the optional substituent(s) is/are halogen(s).

7. A compound according to claim 6 wherein R₁ and R₂ together form cyclohexyl.

8. A compound according to claim 6 wherein R₁ and R₂ together form 5,6-difluorocyclohexyl.

9. A compound according to claim 6 wherein R₁ and R₂ together form 5-chloro-6-fluorocyclohexyl.

10. A compound according to claim 1 wherein R₁ and R₂ together form optionally substituted cycloalkenyl.

11. A compound according to claim 1 wherein R₁ and R₂ together form cyclohexenyl.

12. A compound according to claim 1 wherein R₁ is 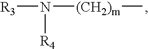
wherein R₃ is alkyl, R₄ is hydrogen and m is 1.

13. A compound according to claim 12 wherein R₃ is methyl.

14. A compound according to claim 12 wherein R₃ is isopropyl.

15. A compound according to claim 1 wherein R₁ is 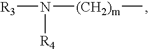
wherein R₃ is cycloalkyl, R₄ is hydrogen and m is 1.

16. A compound according to claim 15 wherein R₃ is cyclopropyl.

17. A compound according to claim 15 wherein R₃ is cyclobutyl.

18. A compound according to claim 1 wherein R₁ is 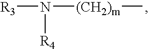
wherein R₃ is cycloalkyl, R₄ is alkyl and m is 1.

19. A compound according to claim 18 wherein R₃ is cyclopropyl, and R₄ is methyl.

20. A compound according to claim 1 wherein R₂ is hydrogen.

21. A compound according to claim 1 wherein R₂ is alkyl.

22. A compound according to claim 21 wherein R₂ is methyl.

23. A compound according to claim 1 wherein R₂ is 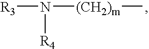
wherein R₃ is optionally substituted alkyl wherein the optional substituent(s) is/are cycloalkyl, R₄ is hydrogen and m is 0.

24. A compound according to claim 23 wherein R₃ is methyl.

25. A compound according to claim 23 wherein R₃ is isopropyl.

26. A compound according to claim 23 wherein R₃ is cyclopropylmethyl.

27. A compound according to claim 1 wherein R₂ is 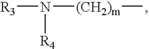
wherein R₃ is optionally substituted alkyl wherein the optional substituent(s) is/are alkynyl, R₄ is hydrogen and m is 0.

28. A compound according to claim 27 wherein R₃ is prop-2-ynyl.

29. A compound according to claim 1 wherein R₂ is 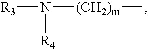
R₃ is cycloalkyl, R₄ is hydrogen and m is 0.

30. A compound according to claim 29 wherein R₃ is cyclopropyl.

31. A compound according to claim 29 wherein R₃ is cyclobutyl, R₄ is hydrogen.

32. A compound according to claim 1 wherein R₂ is 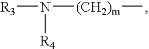
wherein R₃ is cycloalkyl, R₄ is alkyl and m is 0.

33. A compound according to claim 32 wherein R₃ is cyclopropyl, R₄ is methyl and m is 0.

34. A compound according to claim 1 wherein R is 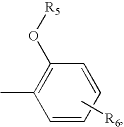
wherein R₅ is cycloalkyl, optionally substituted alkyl wherein the substituent(s) is/are selected from halogen(s) and cycloalkyl and R₆ is selected from hydrogen, halogen and alkyl.

35. A compound according to claim 1 wherein R is selected from 5-fluoro-2-propoxy-phenyl, 2,2,3,3-tetrafluoro-propoxy-phenyl, 2-cyclopentyloxy-5-fluoro-phenyl, 2-methoxy-5-methyl-phenyl, 5-fluoro-2-(2,2,2-trifluoro-ethoxy)-phenyl, 2,2,2-trifluoro-ethoxy-phenyl, 2-cyclopentyloxy-phenyl, 5-fluoro-2-isopropoxy-phenyl, 3-fluoro-2-isopropoxy-phenyl, 5-fluoro-2-methoxy-phenyl, 5-fluoro-2-trifluoromethoxy-phenyl, 2-methoxy-phenyl, 2-isopropoxy-phenyl, 4-fluoro-2-methoxy-phenyl, 4-fluoro-2-(2,2,2-trifluoro-ethoxy)-phenyl, 4-fluoro-2-isopropoxy-phenyl, 3-fluoro-2-methoxy-phenyl and 2-cyclopropylmethoxy-phenyl.

36. A compound, which is:
1-{3-[4-(5-Fluoro-2-propoxy-phenyl)-piperazin-1-yl]-propyl}-3-methyl-4-methylamino-pyrrolidine-2,5-dione (Compound No. 1)
2-(3-{4-[2-(2,2,3,3-Tetrafluoro-propoxy)-phenyl]-piperazin-1-yl}-propyl)-hexahydro-isoindole-1,3-dione (Compound No. 3)
1-{3-[4-(5-Fluoro-2-propoxy-phenyl)-piperazin-1-yl]-propyl}-3-cyclopropylaminomethyl-pyrrolidine-2,5-dione (Compound No. 5)
2-{3-[4-(2-Cyclopentyloxy-5-fluoro-phenyl)-piperazin-1-yl]-propyl}-5,6-difluoro-hexahydro-isoindole-1,3-dione (Compound No. 7)
1-{3-[4-(2-Methoxy-5-methyl-phenyl)-piperazin-1-yl]-propyl}-3,4-dimethyl-pyrrolidine-2,5-dione (Compound No. 9)
1-{3-[4-(2-Methoxy-5-methyl-phenyl)-piperazin-1-yl]-propyl}-3-methyl-4-methylamino-pyrrolidine-2,5-dione (Compound No. 11)
1-{3-[4-(2-Methoxy-5-methyl-phenyl)-piperazin-1-yl]-propyl}-3-cyclopropylamino-4-methyl-pyrrolidine-2,5-dione (Compound No. 13)
1-{3-[4-(5-Fluoro-2-propoxy-phenyl)-piperazin-1-yl]-propyl}-3-cyclobutylamino-4-methyl-pyrrolidine-2,5-dione (Compound No. 15)
1-(3-{4-[5-Fluoro-2-(2,2,2-trifluoro-ethoxy)-phenyl]-piperazin-1-yl}-propyl)-3-cyclopropylamino-4-methyl-pyrrolidine-2,5-dione (Compound No. 17)
1-(3-{4-[5-Fluoro-2-(2,2,2-trifluoro-ethoxy)-phenyl]-piperazin-1-yl}-propyl)-3-methyl-4-methylamino-pyrrolidine-2,5-dione (Compound No. 19)
1-(3-{4-[5-Fluoro-2-(2,2,2-trifluoro-ethoxy)-phenyl]-piperazin-1-yl}-propyl)-3,4-dimethyl-pyrrolidine-2,5-dione (Compound No. 21)
1-(3-{4-[2-(2,2,3,3-Tetrafluoro-propoxy)-phenyl]-piperazin-1-yl}-propyl)-3,4-dimethyl-pyrrolidine-2,5-dione (Compound No. 23)
1-(3-{4-[2-(2,2,3,3-Tetrafluoro-propoxy)-phenyl]-piperazin-1-yl}-propyl)-3-cyclopropylamino-4-methyl-pyrrolidine-2,5-dione (Compound No. 25)
1-(3-{4-[2-(2,2,3,3-Tetrafluoro-propoxy)-phenyl]-piperazin-1-yl}-propyl)-3-cyclobutylamino-4-methyl-pyrrolidine-2,5-dione (Compound No. 27)
1-{3-[4-(2-Cyclopentyloxy-phenyl)-piperazin-1-yl]-propyl}-3-cyclobutylamino-4-methyl-pyrrolidine-2,5-dione (Compound No. 29)
2-{3-[4-(2-Cyclopentyloxy-5-fluoro-phenyl)-piperazin-1-yl]-propyl}-hexahydro-isoindole-1,3-dione (Compound No. 31)
1-{3-[4-(5-Fluoro-2-isopropoxy-phenyl)-piperazin-1-yl]-propyl}-3-methyl-4-methyl amino-pyrrolidine-2,5-dione (Compound No. 33)
1-{3-[4-(5-Fluoro-2-methoxy-phenyl)-piperazin-1-yl]-propyl}-3-methyl-4-methylamino-pyrrolidine-2,5-dione (Compound No. 35)
1-{3-[4-(2-Cyclopropylmethoxy-phenyl)-piperazin-1-yl]-propyl}-3,4-dimethyl-pyrrolidine-2,5-dione (Compound No. 37)
2-{3-[4-(5-Fluoro-2-isopropoxy-phenyl)-piperazin-1-yl]-propyl}-hexahydro-isoindole-1,3-dione (Compound No. 39)
2-{3-[4-(2-Cyclopentyloxy-5-fluoro-phenyl)-piperazin-1-yl]-propyl}-3a,4,7,7a-tetrahydro-isoindole-1,3-dione (Compound No. 41)
1-{3-[4-(2-Cyclopropylmethoxy-phenyl)-piperazin-1-yl]-propyl}-3-cyclopropylamino-4-methyl-pyrrolidine-2,5-dione (Compound No. 43)
2-{3-[4-(5-Fluoro-2-isopropoxy-phenyl)-piperazin-1-yl]-propyl}-3a,4,7,7a-tetrahydro-isoindole-1,3-dione (Compound No. 45)
1-{3-[4-(5-Fluoro-2-isopropoxy-phenyl)-piperazin-1-yl]-propyl}-3-cyclobutylamino-4-methyl-pyrrolidine-2,5-dione (Compound No. 47)
1-{3-[4-(3-Fluoro-2-isopropoxy-phenyl)-piperazin-1-yl]-propyl}-3-methyl-4-methylamino-pyrrolidine-2,5-dione (Compound No. 49)
1-{3-[4-(2-Cyclopropylmethoxy-phenyl)-piperazin-1-yl]-propyl}-3-isopropylamino-4-methyl-pyrrolidine-2,5-dione (Compound No. 51)
1-{3-[4-(2-Cyclopentyloxy-5-fluoro-phenyl)-piperazin-1-yl]-propyl}-3,4-dimethyl-pyrrolidine-2,5-dione (Compound No. 53)
1-{3-[4-(2-Cyclopentyloxy-5-fluoro-phenyl)-piperazin-1-yl]-propyl}-3-cyclopropylamino-4-methyl-pyrrolidine-2,5-dione (Compound No. 55)
1-{3-[4-(5-Fluoro-2-methoxy-phenyl)-piperazin-1-yl]-propyl}-3-(cyclopropylmethyl-ammo)-methyl-pyrrolidine-2,5-dione (Compound No. 57)
1-{3-[4-(5-Fluoro-2-isopropoxy-phenyl)-piperazin-1-yl]-propyl}-3-(cyclopropylmethyl-amino)-4-methyl-pyrrolidine-2,5-dione (Compound No. 59)
1-{3-[4-(5-Fluoro-2-trifluoromethoxy-phenyl)-piperazin-1-yl]-propyl}-3-cyclopropylamino-4-methyl-pyrrolidine-2,5-dione (Compound No. 61)
1-{3-[4-(2-Methoxy-phenyl)-piperazin-1-yl]-propyl}-3-(cyclopropylmethyl-amino)-4-methyl-pyrrolidine-2,5-dione (Compound No. 63)
1-{3-[4-(2-Isopropoxy-phenyl)-piperazin-1-yl]-propyl}-3-isopropylamino-4-methyl-pyrrolidine-2,5-dione (Compound No. 65)
1-{3-[4-(2-Isopropoxy-phenyl)-piperazin-1-yl]-propyl}-3-cyclopropylamino-4-methyl-pyrrolidine-2,5-dione (Compound No. 67)
1-{3-[4-(2-Methoxy-phenyl)-piperazin-1-yl]-propyl}-3-isopropylamino-4-methyl-pyrrolidine-2,5-dione (Compound No. 69)
1-{3-[4-(5-Fluoro-2-isopropoxy-phenyl)-piperazin-1-yl]-propyl}-3,4-dimethyl-pyrrole-2, 5-dione (Compound No. 71)
1-{3-[4-(5-Fluoro-2-isopropoxy-phenyl)-piperazin-1-yl]-propyl}-3,4-dimethyl-pyrrolidine-2, 5-dione (Compound No. 73)
1-{3-[4-(5-Fluoro-2-isopropoxy-phenyl)-]-piperazin-1-yl}-propyl}-3-cyclopropylamino-4-methyl-pyrrolidine-2,5-dione (Compound No. 75)
1-{3-[4-(5-Fluoro-2-isopropoxy-phenyl)-piperazin-1-yl]-propyl}-3-isopropylamino-4-methyl-pyrrolidine-2,5-dione (Compound No. 77)
1-{3-[4-(5-Fluoro-2-isopropoxy-phenyl)-piperazin-1-yl]-propyl}-3-(cyclopropyl-methyl-amino)-4-methyl-pyrrolidine-2,5-dione (Compound No. 79)
1-(3-{4-[2-(2,2,2-Trifluoro-ethoxy)-phenyl]-piperazin-1-yl}-propyl)-3,4-dimethyl-pyrrole-2,5-dione (Compound No. 81)
1-(3-{4-[2-(2,2,2-Trifluoro-ethoxy)-phenyl]-piperazin-1-yl}-propyl)-3,4-dimethyl-pyrrolidine-2,5-dione (Compound No. 83)
1-{3-[4-(2-Cyclopentyloxy-phenyl)-piperazin-1-yl]-propyl}-3,4-dimethyl-pyrrole-2, 5-dione (Compound No. 85)
1-{3-[4-(2-Cyclopentyloxy-phenyl)-piperazin-1-yl]-propyl}-3,4-dimethyl-pyrrolidine-2, 5-dione (Compound No. 87)
1-{3-[4-(2-Cyclopentyloxy-phenyl)-piperazin-1-yl]-propyl}-3-isopropylamino-4-methyl-pyrrolidine-2,5-dione (Compound No. 89)
1-{3-[4-(5-Fluoro-2-methoxy-phenyl)-piperazin-1-yl]-propyl}-3-cyclopropylamino-4-methyl-pyrrolidine-2,5-dione (Compound No. 91)
1-{3-[4-(2-Cyclopentyloxy-phenyl)-piperazin-1-yl]-propyl}-3-methyl-4-methylamino-pyrrolidine-2,5-dione (Compound No. 93)
1-{3-[4-(5-Fluoro-2-methoxy-phenyl)-piperazin-1-yl]-propyl}-3,4-dimethyl-pyrrolidine-2, 5-dione (Compound No. 95)
1-{3-[4-(3-Fluoro-2-methoxy-phenyl)-piperazin-1-yl]-propyl}-3,4-dimethyl-pyrrole-2, 5-dione (Compound No. 97)
1-{3-[4-(3-Fluoro-2-methoxy-phenyl)-piperazin-1-yl]-propyl}-3,4-dimethyl-pyrrolidine-2, 5-dione (Compound No. 99)
1-{3-[4-(3-Fluoro-2-methoxy-phenyl)-piperazin-1-yl]-propyl}-3-methyl-4-methylamino-pyrrolidine-2,5-dione (Compound No. 101)
1-{3-[4-(3-Fluoro-2-methoxy-phenyl)-piperazin-1-yl]-propyl}-3-cyclopropylamino-4-methyl-pyrrolidine-2,5-dione (Compound No. 103)
1-(3-[4-(3-Fluoro-2-isopropoxy-phenyl)-piperazin-1-yl]-propyl}-3,4-dimethyl-pyrrolidine-2, 5-dione (Compound No. 105)
1-{3-[4-(3-Fluoro-2-isopropoxy-phenyl)-piperazin-1-yl]-propyl}-3-cyclopropylamino-4-methyl-pyrrolidine-2,5-dione (Compound No. 107)
2-(3-{4-[2-(2,2,3,3-Tetrafluoro-propoxy)-phenyl]-piperazin-1-yl}-propyl)-3a, 4,7,7a-tetrahydro-isoindole-1,3-dione (Compound No. 109)
2-{4-[4-[2-Isopropoxy-phenyl]-piperazin-1-yl]-butyl}-3a,4,7,7a-tetrahydro-isoindole-1, 3-dione (Compound No. 111)
2-{3-[4-(4-Fluoro-2-methoxy-phenyl)-piperazin-1-yl]-propyl}-3a,4,7,7a-tetrahydro-isoindole-1,3-dione (Compound No. 113)
2-(3-{4-[4-Fluoro-2-(2,2,2-trifluoro-ethoxy)-phenyl]-piperazin-1-yl}-propyl)-3a, 4,7,7a-tetrahydro-isoindole-1,3-dione (Compound No. 115)
2-{3-[4-(4-Fluoro-2-isopropoxy-phenyl)-piperazin-1-yl]-propyl}-3a,4,7,7a-tetrahydro-isoindole-1,3-dione (Compound No. 117)
2-{3-[4-(2-Isopropoxy-phenyl)-piperazin-1-yl]-propyl}-5-chloro-6-fluoro-hexahydro-isoindole-1, 3-dione (Compound No. 119)
1-{3-[4-(5-Fluoro-2-propoxy-phenyl)-piperazin-1-yl]-propyl}-3-methyl-pyrrolidine-2, 5-dione (Compound No. 121)
1-{3-[4-(5-Fluoro-2-propoxy-phenyl)-piperazin-1-yl]-propyl}-3-cyclopropylamino-methyl-pyrrolidine-2,5-dione (Compound No. 123)
1-{3-[4-(2-methoxy-5-methyl-phenyl)-piperazin-1-yl]-propyl}-3-cyclopropylamino methyl-pyrrolidine-2,5-dione (Compound No. 125)
1-(3-{4-[5-Fluoro-2-(2,2,2-trifluoro-ethoxy)-phenyl]-piperazin-1-yl}-propyl)-3-cyclopropylaminomethyl-pyrrolidine-2,5-dione (Compound No. 127)
1-(3-{4-[2-(2,2,3,3-Tetrafluoro-propoxy)-)-phenyl]-piperazin-1-yl}-propyl)-3-cyclopropylaminomethyl-pyrrolidine-2,5-dione (Compound No. 129)
1-(3-{4-[2-(2,2,3,3-tetrafluoro-propoxy)-phenyl]-piperazin-1-yl}-propyl)-3-cyclobutylamino-methyl-pyrrolidine-2,5-dione (Compound No. 131)
1-{3-[4-(3-Fluoro-2-methoxy-phenyl)-piperazin-1-yl]-propyl}-3-methyl-pyrrolidine-2, 5-dione (Compound No. 133)
5-{3-[4-(5-Fluoro-2-isopropoxy-phenyl)-piperazin-1-yl]-propyl}-5-aza-spiro[2.4]heptane-4,6-dione (Compound No. 135)
1-{3-[4-(5-Fluoro-2-isopropoxy-phenyl)-piperazin-1-yl]-propyl}-3-methylaminomethyl-pyrrolidine-2,5-dione (Compound No. 137)
1-{3-[4-(2-Cyclopropylmethoxy-phenyl)-piperazin-1-yl]-propyl}-3-cyclopropylamino-methyl-pyrrolidine-2,5-dione (Compound No. 139)
1-{3-[4-(2-Cyclopropylmethoxy-phenyl)-piperazin-1-yl]-propyl}-3-methylamino-methyl-pyrrolidine-2,5-dione (Compound No. 141)
1-{3-[4-(2-Cyclopropylmethoxy-phenyl)-propyl}-3-methyl-pyrrolidine-2,5-dione (Compound No. 143)
1-{3-[4-(2-Methoxy-phenyl)-piperazin-1-yl]-propyl}-3-methylaminomethyl-pyrrolidine-2,5-dione (Compound No. 145)
1-{3-[4-(2-Cyclopentyloxy-phenyl)-piperazin-1-yl]-propyl}-3-cyclopropylaminomethyl-pyrrolidine-2,5-dione (Compound No. 147)
1-{3-[4-(2-Cyclopentyloxy-phenyl)-piperazin-1-yl]-propyl}-3-(isopropylamino-methyl)-pyrrolidine-2, 5-dione (Compound No. 149)
5-{3-[4-(2-Cyclopentyloxy-phenyl)-piperazin-1-yl]-propyl}-5-aza-spiro[2,4]heptane-4,6-dione (Compound No. 151)
1-{3-[4-(3-Fluoro-2-isopropoxy-phenyl)-piperazin-1-yl]-propyl}-3-(isopropylamino-methyl)-pyrrolidine-2,5-dione (Compound No. 153)
1-{3-[4-(3-Fluoro-2-isopropoxy-phenyl)-piperazin-1-yl]-propyl}-3-cyclopropylamino-methyl-pyrrolidine-2,5-dione (Compound No. 155)
5-{3-[4-(3-Fluoro-2-isopropoxy-phenyl)-piperazin-1-yl]-propyl}-5-aza-spiro[2,4]heptane-4,6-dione (Compound No. 157)
1-{3-[4-(5-Fluoro-2-isopropoxy-phenyl)-piperazin-1-yl]-propyl}-3-cyclopropylamino-methyl-pyrrolidine-2,5-dione (Compound No. 159)
1-{3-[4-(5-Fluoro-2-isopropoxy-phenyl)-piperazin-1-yl]-propyl}-3-(isopropylamino-methyl)-pyrrolidine-2,5-dione (Compound No. 161)
1-{3-[4-(2-Cyclopentyloxy-5-fluoro-phenyl)-piperazin-1-yl]-propyl}-3-[(cyclopropyl-methyl-amino)-methyl]-pyrrolidine-2,5-dione (Compound No. 163)
1-{3-[4-(2-Cyclopentyloxy-5-fluoro-phenyl)-piperazin-1-yl]-propyl}-3-isopropylamino-methyl)-pyrrolidine-2,5-dione (Compound No. 165)
1-{3-[4-(2-Cyclopentyloxy-5-fluoro-phenyl)-piperazin-1-yl]-propyl}-3-cyclopropylaminomethyl-pyrrolidine-2,5-dione (Compound No. 167)
1-{3-[4-(2-Cyclopentyloxy-5-fluoro-phenyl)-piperazin-1-yl]-propyl}-3-methyl-pyrrolidine-2,5-dione (Compound No. 169)
1-{3-[4-(2-Cyclopentyloxy-5-fluoro-phenyl)-piperazin-1-yl]-propyl}-3-methyl-pyrrole-2,5-dione (Compound No. 171)
1-{3-[4-(2-Methoxy-phenyl)-piperazin-1-yl]-propyl}-3-methyl-4-prop-2-ynylamino-pyrrolidine-2,5-dione (Compound No. 173)
1-{3-[4-(4-Fluoro-2-isopropoxy-phenyl)-piperazin-1-yl]-propyl}-3-[(cyclopropyl-methyl-amino)-methyl]-pyrrolidine-2,5-dione (Compound No. 175)
1-{3-[4-(2-Methoxy-phenyl)-piperazin-1-yl]-propyl}-3-[(cyclopropyl-methyl-amino)-methyl]-pyrrolidine-2,5-dione (Compound No. 177)
1-(3-{4-(2-(2,2,2-Trifluoro-ethoxy)-phenyl]-piperazin-1-yl}-propyl)-3-[(cyclopropyl-methyl-amino)-methyl]-pyrrolidine-2,5-dione (Compound No. 179)
1-{3-[4-(2-Methoxy-phenyl)-piperazin-1-yl]-propyl}-3-(isopropylamino)-methyl)-pyrrolidine-2,5-dione (Compound No. 181)
5-(3-{4-[2-(2,2,2-Trifluoro-ethoxy)-phenyl]-piperazin-1-yl}-propyl)-5-aza-spiro[2,4]heptane-4,6-dione (Compound No. 183)
1-{3-[4-(5-Fluoro-2-isopropoxy-phenyl)-piperazin-1-yl]-propyl}-3-methyl-pyrrole-2, 5-dione (Compound No. 185)
1-{3-[4-(5-Fluoro-2-isopropoxy-phenyl)-piperazin-1-yl]-propyl}-3-methyl-pyrrolidine-2,5-dione (Compound No. 187)
1-{3-[4-(5-Fluoro-2-methoxy-phenyl)-piperazin-1-yl]-propyl}-3-methyl-pyrrole-2, 5-dione (Compound No. 189)
1-{3-[4-(5-Fluoro-2-methoxy-phenyl)-piperazin-1-yl]-propyl}-3-methyl-pyrrolidine-2,5-dione (Compound No. 191)
1-{3-[4-(2-Cyclopentyloxy-phenyl)-piperazin-1-yl]-propyl}-3-methyl-pyrrolidine-2, 5-dione (Compound No. 193)
1-{3-[4-(3-Fluoro-2-methoxy-phenyl)-piperazin-1-yl]-propyl}-3-methyl-pyrrole-2,5-dione (Compound No. 195)
1-{3-[4-(3-Fluoro-2-isopropoxy-phenyl)-piperazin-1-yl]-propyl}-3-methyl-pyrrolidine-2,5-dione (Compound No. 197)
their pharmaceutically acceptable acid addition salts, solvates, enantiomers, diastereomers, regioisomers, N-oxides, polymorphs, prodrugs and metabolites.

37. A compound, which is:
1-{3-[4-(5-Fluoro-2-propoxy-phenyl)-piperazin-1-yl]-propyl}-3-methyl-4-methylamino-pyrrolidine-2,5-dione hydrochloride salt (Compound No. 2)
2-(3-{4-[2-(2,2,3,3-Tetrafluoro-propoxy)-phenyl]-piperazin-1-yl}-propyl)-hexahydro-isoindole-1,3-dione hydrochloride salt (Compound No. 4)
1-{3-[4-(5-Fluoro-2-propoxy-phenyl)-piperazin-1-yl]-propyl}-3-cyclopropylamino-4-methyl-pyrrolidine-2,5-dione hydrochloride salt (Compound No. 6)
2-{3-[4-(2-Cyclopentyloxy-5-fluoro-phenyl)-piperazin-1-yl)-propyl}-5,6-difluoro-hexahydro-isoindole-1,3-dione hydrochloride salt (Compound No. 8)
1-{3-[4-(2-Methoxy-5-methyl-phenyl)-piperazin-1-yl]-propyl}-3,4-dimethyl-pyrrolidine-2,5-dione hydrochloride salt (Compound No. 10)
1-{3-[4-(2-Methoxy-5-methyl-phenyl)-piperazin-1-yl]-propyl}-3-methyl-4-methylamino-pyrrolidine-2,5-dione hydrochloride salt (Compound No. 12)
1-{3-[4-(2-Methoxy-5-methyl-phenyl)-piperazin-1-yl]-propyl}-3-cyclopropylamino-4-methyl-pyrrolidine-2,5-dione hydrochloride salt (Compound No. 14)
1-{3-[4-(5-Fluoro-2-propoxy-phenyl)-piperazin-1-yl]-propyl}-3-cyclobutylamino-4-methyl-pyrrolidine-2,5-dione hydrochloride salt (Compound No. 16)
1-(3-{4-[5-Fluoro-2-(2,2,2-trifluoro-ethoxy)-phenyl]-piperazin-1-yl}-propyl)-3-cyclopropylamino-4-methyl-pyrrolidine-2,5-dione hydrochloride salt (Compound No. 18)
1-(3-{4-[5-Fluoro-2-(2,2,2-trifluoro-ethoxy)-phenyl]-piperazin-1-yl}-propyl)-3-methyl-4-methylamino-pyrrolidine-2,5-dione hydrochloride salt (Compound No. 20)
1-(3-{4-[5-Fluoro-2-(2,2,2-trifluoro-ethoxy)-phenyl]-piperazin-1-yl}-propyl)-3,4-dimethyl-pyrrolidine-2,5-dione hydrochloride salt (Compound No. 22)
1-(3-{4-[2-(2,2,3,3-Tetrafluoro-propoxy)-phenyl]-piperazin-1-yl}-propyl)-3,4-dimethyl-pyrrolidine-2,5-dione hydrochloride salt (Compound No. 24)
1-(3-{4-[2-(2,2,3,3-Tetrafluoro-propoxy)-phenyl]-piperazin-1-yl}-propyl)-3-cyclopropylamino-4-methyl-pyrrolidine-2,5-dione hydrochloride salt (Compound No. 26)
1-(3-{4-[2-(2,2,3,3-Tetrafluoro-propoxy)-phenyl]-piperazin-1-yl}-propyl)-3-cyclobutylamino-4-methyl-pyrrolidine-2,5-dione hydrochloride salt (Compound No. 28)
1-{3-[4-(2-Cyclopentyloxy-phenyl)-piperazin-1-yl]-propyl}-3-cyclobutylamino-4-methyl-pyrrolidine-2,5-dione hydrochloride salt (Compound No. 30)
2-{3-[4-(2-Cyclopentyloxy-5-fluoro-phenyl)-piperazin-1-yl]-propyl}-hexahydro-isoindole-1,3-dione hydrochloride salt (Compound No. 32)
1-{3-[4-(5-Fluoro-2-isopropoxy-phenyl)-piperazin-1-yl]-propyl}-3-methyl-4-methyl amino-pyrrolidine-2,5-dione hydrochloride salt (Compound No. 34)
1-{3-[4-(5-Fluoro-2-methoxy-phenyl)-piperazin-1-yl]-propyl}-3-methyl-4-methylamino-pyrrolidine-2,5-dione hydrochloride salt (Compound No. 36)
1-{3-[4-(2-Cyclopropylmethoxy-phenyl)-piperazin-1-yl]-propyl}-3,4-dimethyl-pyrrolidine-2,5-dione hydrochloride salt (Compound No. 38)
2-{3-[4-(5-Fluoro-2-isopropoxy-phenyl)-piperazin-1-yl]-propyl}-hexahydro-isoindole-1,3-dione hydrochloride salt (Compound No. 40)
2-{3-[4-(2-Cyclopentyloxy-5-fluoro-phenyl)-piperazin-1-yl]-propyl}-3a,4,7,7a-tetrahydro-isoindole-1,3-dione hydrochloride salt (Compound No. 42)
1-{3-[4-(2-Cyclopropylmethoxy-phenyl)-piperazin-1-yl]-propyl}-3-cyclopropylamino-4-methyl-pyrrolidine-2,5-dione hydrochloride salt (Compound No. 44)
2-{3-[4-(5-Fluoro-2-isopropoxy-phenyl)-piperazin-1-yl]-propyl}-3a,4,7,7a-tetrahydro-isoindole-1,3-dione hydrochloride salt (Compound No. 46)
1-{3-[4-(5-Fluoro-2-isopropoxy-phenyl)-piperazin-1-yl]-propyl}-3-cyclobutylamino-4-methyl-pyrrolidin-2,5-dione hydrochloride salt (Compound No. 48)
1-{3-[4-(3-Fluoro-2-isopropoxy-phenyl)-piperazin-1-yl]-propyl}-3-methyl-4-methylamino-pyrrolidine-2,5-dione hydrochloride salt (Compound No. 50)
1-{3-[4-(2-Cyclopropylmethoxy-phenyl)-piperazin-1-yl]-propyl}-3-isopropylamino-4-methyl-pyrrolidine-2,5-dione hydrochloride salt (Compound No. 52)
1-{3-[4-(2-Cyclopentyloxy-5-fluoro-phenyl)-piperazin-1-yl]-propyl}-3,4-dimethyl-pyrrolidine-2,5-dione hydrochloride salt (Compound No. 54)
1-{3-[4-(2-Cyclopentyloxy-5-fluoro-phenyl)-piperazin-1-yl]-propyl}-3-cyclopropylamino-4-methyl-pyrrolidine-2,5-dione hydrochloride salt (Compound No. 56)
1-{3-[4-(5-Fluoro-2-methoxy-phenyl)-piperazin-1-yl]-propyl}-3-(cyclopropylmethyl-amino)-4-methyl-pyrrolidine-2,5-dione hydrochloride salt (Compound No. 58)
1-{3-[4-(5-Fluoro-2-isopropoxy-phenyl)-piperazin-1-yl]-propyl}-3-(cyclopropylmethyl-amino)-4-methyl-pyrrolidine-2,5-dione hydrochloride salt (Compound No. 60)
1-{3-[4-(5-Fluoro-2-trifluoromethoxy-phenyl)-piperazin-1-yl]-propyl}-3-cyclopropylamino-4-methyl-pyrrolidine-2,5-dione hydrochloride salt (Compound No. 62)
1-{3-[4-(2-Methoxy-phenyl)-piperazin-1-yl]-propyl}-3-(cyclopropylmethyl-amino)-4-methyl-pyrrolidine-2,5-dione hydrochloride salt (Compound No. 64)
1-{3-[4-(2-Isopropoxy-phenyl)-piperazin-1-yl]-propyl}-3-isopropylamino-4-methyl-pyrrolidine-2,5-dione hydrochloride salt (Compound No. 66)
1-{3-[4-(2-Isopropoxy-phenyl)-piperazin-1-yl]-propyl}-3-cyclopropylamino-4-methyl-pyrrolidine-2,5-dione hydrochloride salt (Compound No. 68)
1-{3-[4-(2-Methoxy-phenyl)-piperazin-1-yl]-propyl}-3-isopropylamino-4-methyl-pyrrolidine-2,5-dione hydrochloride salt (Compound No. 70)
1-{3-[4-(5-Fluoro-2-isopropoxy-phenyl)-piperazin-1-yl]-propyl}-3,4-dimethyl-pyrrole-2, 5-dione hydrochloride salt (Compound No. 72)
1-{3-[4-(5-Fluoro-2-isopropoxy-phenyl)-piperazin-1-yl]-propyl}-3,4-diethyl-pyrrolidine-2, 5-dione hydrochloride salt (Compound No. 74)
1-{3-[4-(5-Fluoro-2-isopropoxy-phenyl)-]-piperazin-1-yl]-propyl}-3-cyclopropylaminomethyl-pyrrolidine-2,5-dione hydrochloride salt (Compound No. 76)
1-{3-[4-(5-Fluoro-2-isopropoxy-phenyl)-piperazin-1-yl]-propyl}-3-isopropylamino-4-methyl-pyrrolidine-2,5-dione hydrochloride salt (Compound No. 78)
1-{3-[4-(5-Fluoro-2-isopropoxy-phenyl)-piperazin-1-yl]-propyl}-3-(cyclopropyl-methyl-amino)-4-methyl-pyrrolidine-2,5-dione hydrochloride salt (Compound No. 80)
1-(3-{4-[2-(2,2,2-Trifluoro-ethoxy)-phenyl]-piperazin-1-yl}-propyl)-3,4-dimethyl-pyrrole-2,5-dione hydrochloride salt (Compound No. 82)
1-(3-{4-[2-(2,2,2-Trifluoro-ethoxy)-phenyl]-piperazin-1-yl}-propyl)-3,4-dimethyl-pyrrolidine-2,5-dione hydrochloride salt (Compound No. 84)
1-{3-[4-(2-Cyclopentyloxy-phenyl)-piperazin-1-yl]-propyl}-3,4-dimethyl-pyrrole-2, 5-dione hydrochloride salt (Compound No. 86)
1-{3-[4-(2-Cyclopentyloxy-phenyl)-piperazin-1-yl]-propyl}-3,4-dimethyl-pyrrolidine-2, 5-dione hydrochloride salt (Compound No. 88)
1-{3-[4-(2-Cyclopentyloxy-phenyl)-piperazin-1-yl]-propyl}-3-isopropylamino-4-methyl-pyrrolidine-2,5-dione hydrochloride salt (Compound No. 90)
1-{3-[4-(5-Fluoro-2-methoxy-phenyl)-piperazin-1-yl]-propyl}-3-cyclopropylamino-4-methyl-pyrrolidine-2,5-dione hydrochloride salt (Compound No. 92)
1-{3-[4-(2-Cyclopentyloxy-phenyl)-piperazin-1-yl]-propyl}-3-methyl-methylamino-pyrrolidine-2, 5-dione hydrochloride salt (Compound No. 94)
1-{3-[4-(5-Fluoro-2-methoxy-phenyl)-piperazin-1-yl]-propyl}-3,4-dimethyl-pyrrolidine-2, 5-dione hydrochloride salt (Compound No. 96)
1-{3-[4-(3-Fluoro-2-methoxy-phenyl)-piperazin-1-yl]-propyl}-3,4-dimethyl-pyrrole-2, 5-dione hydrochloride salt (Compound No. 98)
1-{3-[4-(3-Fluoro-2-methoxy-phenyl)-piperazin-1-yl]-propyl}-3,4-dimethyl-pyrrolidine-2, 5-dione hydrochloride salt (Compound No. 100)
1-{3-[4-(3-Fluoro-2-methoxy-phenyl)-piperazin-1-yl]-propyl}-3-methyl-4-methylamino-pyrrolidine-2,5-dione hydrochloride salt (Compound No. 102)
1-{3-[4-(3-Fluoro-2-methoxy-phenyl)-piperazin 1-yl]-propyl}-3-cyclopropylamino-4-methyl-pyrrolidine-2,5-dione hydrochloride salt (Compound No. 104)
1-{3-[4-(3-Fluoro-2-isopropoxy-phenyl)-piperazin-1-yl]-propyl}-3,4-dimethyl-pyrrolidine-2, 5-dione hydrochloride salt (Compound No. 106)
1-{3-[4-(3-Fluoro-2-isopropoxy-phenyl)-piperazin-1-yl]-propyl}-3-cyclopropylamino-4-methyl-pyrrolidine-2,5-dione hydrochloride salt (Compound No. 108)
2-(3-{4-[2-(2,2,3,3-Tetrafluoro-propoxy)-phenyl]-piperazin-1-yl}-propyl)-3a, 4,7,7a-tetrahydro-isoindole-1,3-dione hydrochloride salt (Compound No. 110)
2-{4-[4-[2-Isopropoxy-phenyl]-piperazin-1-yl]-butyl}-3a,4,7,7a-tetrahydro-isoindole-1, 3-dione hydrochloride salt (Compound No. 112)
2-{3-[4-(4-Fluoro-2-methoxy-phenyl)-piperazin-1-yl]-propyl}-3a,4,7,7a-tetrahydro-isoindole-1,3-dione hydrochloride salt (Compound No. 114)
2-(3-{4-[4-Fluoro-2-(2,2,2-trifluoro-ethoxy)-phenyl]-piperazin-1-yl}-propyl)-3a, 4,7,7a-tetrahydro-isoindole-1,3-dione hydrochloride salt (Compound No. 116)
2-{3-[4-(4-Fluoro-2-isopropoxy-phenyl)-piperazin-1-yl]-propyl}-3a,4,7,7a-tetrahydro-isoindole-1,3-dione hydrochloride salt (Compound No. 118)
2-{3-[4-(2-Isopropoxy-phenyl)-piperazin-1-yl]-propyl}-5-chloro-6-fluoro-hexahydro-isoindole-1, 3-dione hydrochloride salt (Compound No. 120)
1-{3-[4-(5-Fluoro-2-propoxy-phenyl)-piperazin-1-yl]-propyl}-3-methyl-pyrrolidine-2, 5-dione hydrochloride salt (Compound No. 122)
1-{3-[4-(5-Fluoro-2-propoxy-phenyl)-piperazin-1-yl]-propyl}-3-cyclopropylamino-methyl-pyrrolidine-2,5-dione hydrochloride salt (Compound No. 124)
1-{3-[4-(2-methoxy-5-methyl-phenyl)-piperazin-1-yl]-propyl}-3-cyclopropylamino methyl-pyrrolidine-2,5-dione hydrochloride salt (Compound No. 126)
1-(3-{4-[5-Fluoro-2-(2,2,2-trifluoro-ethoxy)-phenyl]-piperazin-1-yl}-propyl)-3-cyclopropylaminomethyl-pyrrolidine-2,5-dione hydrochloride salt (Compound No. 128)
1-(3-{4-[2-(2,2,3,3-Tetrafluoro-propoxy)-phenyl]-piperazin-1-yl}-propyl)-3-cyclopropylaminomethyl-pyrrolidine-2,5-dione hydrochloride salt (Compound No. 130)
1-(3-{4-[2-(2,2,3,3-tetrafluoro-propoxy)-phenyl]-piperazin-1-yl}-propyl)-3-cyclobutylamino-methyl-pyrrolidine-2,5-dione hydrochloride salt (Compound No. 132)
1-{3-[4-(3-Fluoro-2-methoxy-phenyl)-piperazin-1-yl]-propyl}-3-methyl-pyrrolidine-2, 5-dione hydrochloride salt (Compound No. 134)
5-{3-[4-(5-Fluoro-2-isopropoxy-phenyl)-piperazin-1-yl]-propyl}-5-aza-spiro[2.4]heptane-4,6-dione hydrochloride salt (Compound No. 136)
1-{3-[4-(5-Fluoro-2-isopropoxy-phenyl)-piperazin-1-yl]-propyl}-3-methylaminomethyl-pyrrolidine-2,5-dione hydrochloride salt (Compound No. 138)
1-{3-[4-(2-Cyclopropylmethoxy-phenyl)-piperazin-1-yl]-propyl}-3-cyclopropylamino-methyl-pyrrolidine-2,5-dione hydrochloride salt (Compound No. 140)
1-{3-[4-(2-Cyclopropylmethoxy-phenyl)-piperazin-1-yl]-propyl}-3-methylamino-methyl-pyrrolidine-2,5-dione hydrochloride salt (Compound No. 142)
1-{3-[4-(2-Cyclopropylmethoxy-phenyl)-piperazin-1-yl]-propyl}-3-methyl-pyrrolidine-2,5-dione hydrochloride salt (Compound No. 144)
1-{3-[4-(2-Methoxy-phenyl)-piperazin-1-yl]-propyl}-3-methylaminomethyl-pyrrolidine-2,5-dione hydrochloride salt (Compound No. 146)
1-{3-[4-(2-Cyclopentyloxy-phenyl)-piperazin-1-yl]-propyl}-3-cyclopropylaminomethyl-pyrrolidine-2,5-dione hydrochloride salt (Compound No. 148)
1-{3-[4-(2-Cyclopentyloxy-phenyl)-piperazin-1-yl]-propyl}-3-(isopropylamino-methyl)-pyrrolidine-2, 5-dione hydrochloride salt (Compound No. 150)
5-{3-[4-(2-Cyclopentyloxy-phenyl)-piperazin-1-yl]-propyl}-5-aza-spiro[2,4]heptane-4,6-dione hydrochloride salt (Compound No. 152)
1-{3-[4-(3-Fluoro-2-isopropoxy-phenyl)-piperazin-1-yl]-propyl}-3-(isopropylamino-methyl)-pyrrolidine-2,5-dione hydrochloride salt (Compound No. 154)
1-{3-[4-(3-Fluoro-2-isopropoxy-phenyl)-piperazin-1-yl]-propyl}-3-cyclopropylamino-methyl-pyrrolidine-2,5-dione hydrochloride salt (Compound No. 156)
5-{3-[4-(3-Fluoro-2-isopropoxy-phenyl)-piperazin-1-yl]-propyl}-5-aza-spiro[2,4]heptane-4,6-dione hydrochloride salt (Compound No. 158)
1-{3-[4-(5-Fluoro-2-isopropoxy-phenyl)-piperazin-1-yl]-propyl}-3-cyclopropylamino-methyl-pyrrolidine-2,5-dione hydrochloride salt (Compound No. 160)
1-{3-[4-(5-Fluoro-2-isopropoxy-phenyl)-piperazin-1-yl]-propyl}-3-(isopropylamino-methyl)-pyrrolidine-2,5-dione hydrochloride salt (Compound No. 162)
1-{3-[4-(2-Cyclopentyloxy-5-fluoro-phenyl)-piperazin-1-yl]-propyl}-[(3-cyclopropyl-methyl-amino)-methyl]-pyrrolidine-2,5-dione hydrochloride salt (Compound No. 164)
1-{3-[4-(2-Cyclopentyloxy-5-fluoro-phenyl)-piperazin-1-yl]-propyl}-3-isopropylamino-methyl)-pyrrolidine-2,5-dione hydrochloride salt (Compound No. 166)
1-{3-[4-(2-Cyclopentyloxy-5-fluoro-phenyl)-piperazin-1-yl]-propyl}-3-cyclopropylaminomethyl-pyrrolidine-2,5-dione hydrochloride salt (Compound No. 168)
1-{3-[4-(2-Cyclopentyloxy-5-fluoro-phenyl)-piperazin-1-yl]-propyl}-3-methyl-pyrrolidine-2,5-dione hydrochloride salt (Compound No. 170)
1-{3-[4-(2-Cyclopentyloxy-5-fluoro-phenyl)-piperazin-1-yl]-propyl}-3-methyl-pyrrole-2,5-dione hydrochloride salt (Compound No. 172)
1-{3-[4-(2-Methoxy-phenyl)-piperazin-1-yl]-propyl}-3-methyl-4-prop-2-ynylamino-pyrrolidine-2,5-dione hydrochloride salt (Compound No. 174)
1-{3-[4-(4-Fluoro-2-isopropoxy-phenyl)-piperazin-1-yl]-propyl}-3-[(cyclopropyl-methyl-amino)-methyl]-pyrrolidine-2,5-dione hydrochloride salt (Compound No. 176)
1-{3-[4-(2-methoxy-phenyl)-piperazin-1-yl]-propyl}-3-[(cyclopropyl-methyl-amino)-methyl]-pyrrolidine-2,5-dione hydrochloride salt (Compound No. 178)
1-(3-{4-(2-(2,2,2-Trifluoro-ethoxy)-phenyl]-piperazin-1-yl}-propyl)-3-[(cyclopropyl-methyl-amino)-methyl]-pyrrolidine-2,5-dione hydrochloride salt (Compound No. 180)
1-{3-[4-(2-Methoxy-phenyl)-piperazin-1-yl]-propyl}-3-(isopropylamino)-methyl)-pyrrolidine-2,5-dione hydrochloride salt (Compound No. 182)
5-(3-{4-[2-(2,2,2-Trifluoro-ethoxy)-phenyl]-piperazin-1-yl}-propyl)-5-aza-spiro[2,4]heptane-4,6-dione hydrochloride salt (Compound No. 184)
1-{3-[4-(5-Fluoro-2-isopropoxy-phenyl)-piperazin-1-yl]-propyl}-3-methyl-pyrrole-2, 5-dione hydrochloride salt (Compound No. 186)
1-{3-[4-(5-Fluoro-2-isopropoxy-phenyl)-piperazin-1-yl]-propyl}-3-methyl-pyrrolidine-2,5-dione hydrochloride salt (Compound No. 188)
1-{3-[4-(5-Fluoro-2-methoxy-phenyl)-piperazin-1-yl]-propyl}-3-methyl-pyrrole-2, 5-dione hydrochloride salt (Compound No. 190)
1-{3-[4-(5-Fluoro-2-methoxy-phenyl)-piperazin-1-yl]-propyl}-3-methyl-pyrrolidine-2,5-dione hydrochloride salt (Compound No. 192)
1-{3-[4-(2-Cyclopentyloxy-phenyl)-piperazin-1-yl]-propyl}-3-methyl-pyrrolidine-2, 5-dione hydrochloride salt (Compound No. 194)
1-{3-[4-(3-Fluoro-2-methoxy-phenyl)-piperazin-1-yl]-propyl}-3-methyl-pyrrole-2,5-dione hydrochloride salt (Compound No. 196)
1-{3-[4-(3-Fluoro-2-isopropoxy-phenyl)-piperazin-1-yl]-propyl}-3-methyl-pyrrolidine-2,5-dione hydrochloride salt (Compound No. 198)

38. A pharmaceutical composition comprising a therapeutically effective amount of a compound of claim 1 optionally together with pharmaceutically acceptable carriers, excipients or diluents.

39. A method for treatment of a patient suffering from a disease or disorder mediated through α₁ₐ and/or α_{1d} adrenergic receptor, comprising administering to said patient a therapeutically effective amount of a compound of claim 1.

40. A method for treatment of a patient suffering from disease or disorder mediated through α₁ₐ and/or α_{1d} adrenergic receptor, comprising administering to said patient a therapeutically effective amount of a pharmaceutical composition according to claim 38.

41. The method according to claim 39 or 40 wherein a disease or disorder is benign prostatic hyperplasia.

42. A method for treatment of a patient suffering from lower urinary tract symptoms associated with or without benign prostatic hyperplasia, comprising administering to said patient a therapeutically effective amount of a compound of claim 1.

43. A method according to claim 42 wherein lower urinary tract symptoms are irritative symptoms.

44. A method according to claim 43 wherein irritative symptoms are selected from the group consisting of frequent urination, urgent urination, nocturia and unstable bladder contractions.

45. A method according to claim 42 wherein lower urinary tract symptoms are obstructive symptoms.

46. A method according to claim 45 wherein obstructive symptoms are selected from the group consisting of hesitancy, poor stream, prolong urination, and feelings of incomplete emptying.

47. A method according to claim 42 wherein the said patient is a male.

48. A method according to claim 42 wherein the said patient is a female.

49. A process for the preparation of a compound of Formula VI, 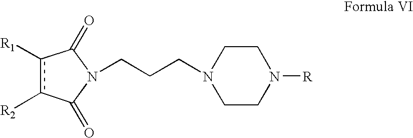
its pharmaceutically acceptable acid addition salts, solvates, enantiomers, diastereomers, regioisomers, N-oxides, polymorphs, prodrugs and metabolites wherein,
— represents no bond or a single bond;
R₁ and R₂ are selected from alkyl, cycloalkyl, or 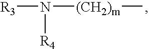
wherein m is the integer 0 or 1;
R₃ is selected from alkyl, or cycloalkyl;
R⁴ is selected from hydrogen or alkyl;
R₂ may also be hydrogen; or
R₁ and R₂ can together form a group selected from cycloalkyl or cycloalkenyl; and
R is 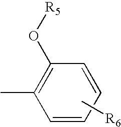
wherein R₅ is selected from alkyl or cycloalkyl, and wherein R₆ is selected from hydrogen, halogen or alkyl,
the method comprising:
reacting a compound of Formula II with acrylonitrile, to give a compound of 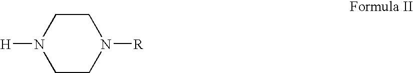
Formula III (wherein R is the same as defined earlier); 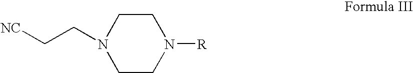
hydrogenating the compound of Formula III to give a compound of Formula IV; and 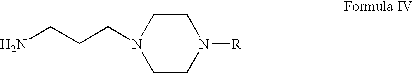
treating the compound of Formula IV with a compound of Formula V, to give compound of Formula VIII (wherein R₁ and R₂ are the same as defined earlier).

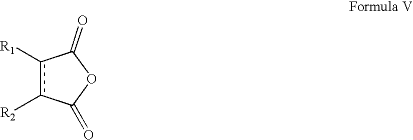

50. A process for the preparation of a compound of Formula X, 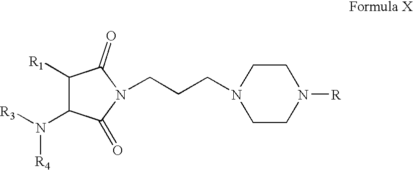
its pharmaceutically acceptable acid addition salts, solvates, enantiomers, diastereomers, regioisomers, N-oxides, polymorphs, prodrugs and metabolites wherein,
R₁ is selected from alkyl, cycloalkyl, or 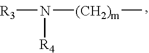
wherein m is the integer 0 or 1;
R₃ is selected from alkyl, or cycloalkyl;
R₄ is selected from hydrogen or alkyl; and
R is 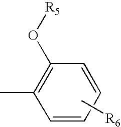
wherein R₅ is selected from alkyl or cycloalkyl, and wherein R₆ is selected from hydrogen, halogen or alkyl,
the method comprising:
reacting a compound of Formula IV with a compound of Formula VIII to give a compound of Formula VIII (wherein R and R₁ are the same as defined earlier); 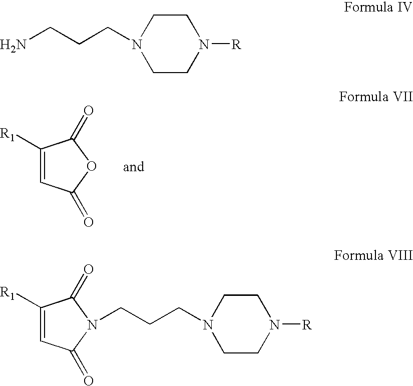
treating the compound of Formula VIII with a compound of Formula IX to give a compound of Formula X,
R₃R₄NH Formula IX

51. A process for the preparation of a compound of Formula XI, 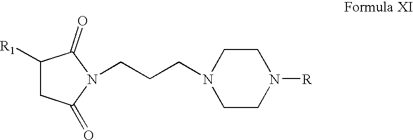
its pharmaceutically acceptable acid addition salts, solvates, enantiomers, diastereomers, regioisomers, N-oxides, polymorphs, prodrugs and metabolites wherein,
R₁ is selected from alkyl, cycloalkyl, or 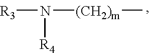
wherein m is the integer 0 or 1;
R₃ is selected from alkyl, or cycloalkyl;
R₄ is selected from hydrogen or alkyl;
R is 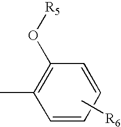
wherein R₅ is selected from alkyl or cycloalkyl, and wherein R₆ is selected from hydrogen, halogen or alkyl,
the method comprising:
reducing a compound of Formula VIII to give a compound of Formula XI.

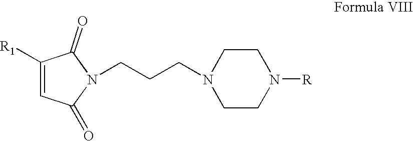

52. A process for the preparation of a compound of Formula XIII, 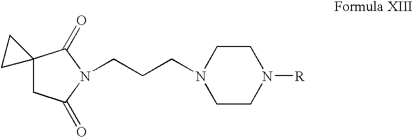
its pharmaceutically acceptable acid addition salts, solvates, enantiomers, diastereomers, regioisomers, N-oxides, polymorphs, prodrugs and metabolites wherein,
R is 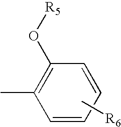
wherein R₅ is selected from alkyl or cycloalkyl, and wherein R₆ is selected from hydrogen, halogen or alkyl,
the method comprising:
reacting a compound of Formula IV with itaconic anhydride, 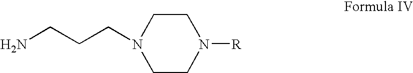
to give a compound of Formula XII (wherein R is the same as defined earlier); and 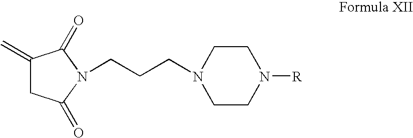
treatment of the compound of Formula XII with a methylene transfer reagent gives a compound of Formula XIII.

53. A process for the preparation of a compound of Formula XIV, 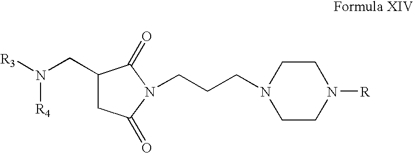
its pharmaceutically acceptable acid addition salts, solvates, enantiomers, diastereomers, regioisomers, N-oxides, polymorphs, prodrugs and metabolites wherein,
R₃ is selected from alkyl, or cycloalkyl;
R₄ is selected from hydrogen or alkyl; and
R is 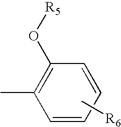
wherein R₅ is selected from alkyl or cycloalkyl, and wherein R₆ is selected from hydrogen, halogen or alkyl,
the method comprising:
treating a compound of Formula XII with a compound of Formula IX, to give a compound of Formula XIV.

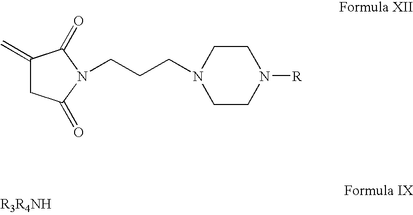

54. A process for the preparation of a compound of Formula XVII, 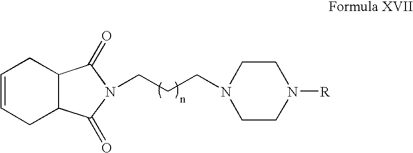
its pharmaceutically acceptable acid addition salts, solvates, enantiomers, diastereomers, regioisomers, N-oxides, polymorphs, prodrugs and metabolites wherein,
the variable n represents the integers 1 or 2; and
R is 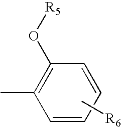
wherein R₅ is selected from alkyl or cycloalkyl, and wherein
R₆ is selected from hydrogen, halogen or alkyl,
the method comprising:
reacting 3α,4,7,7α-tetrahydro-isoindole-1,3-dione with a compound of Formula XV, to give a compound of Formula XVI (wherein X is a halogen and n is the same as defined earlier); and 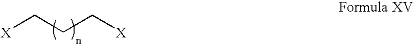
treating the compound of Formula XVI with a compound of Formula I to give a compound of Formula XVII.

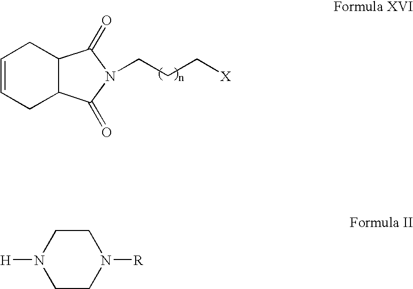

55. A process for the preparation of a compound of Formula XVIII, 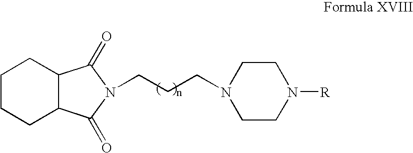
its pharmaceutically acceptable acid addition salts, solvates, enantiomers, diastereomers, regioisomers, N-oxides, polymorphs, prodrugs and metabolites wherein,
the variable n represents the integers 1 or 2; and
R is 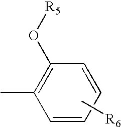
wherein R₅ is selected from alkyl or cycloalkyl, and wherein R₆ is selected from hydrogen, halogen or alkyl,
the method comprising:
hydrogenation of a compound of Formula XVII to give a compound of Formula XVIII.

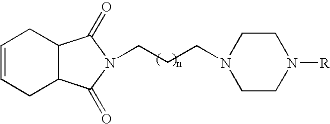

56. A process for the preparation compound of Formula XXII, 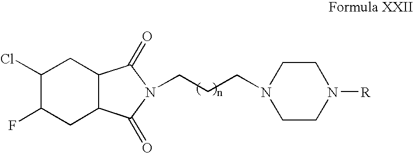
its pharmaceutically acceptable acid addition salts, solvates, enantiomers, diastereomers, regioisomers, N-oxides, polymorphs, prodrugs and metabolites wherein,
the variable n represents the integers 1 or 2; and
R is 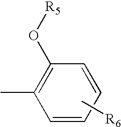
wherein R₅ is selected from alkyl or cycloalkyl, and wherein R₆ is selected from hydrogen, halogen or alkyl,
the method comprising:
reacting a compound of Formula XVI with a peroxyacid, 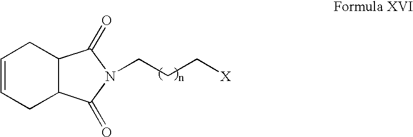
to give a compound of Formula XIX (wherein X is a halogen and n is the same as defined earlier); 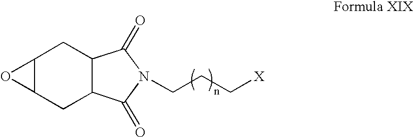
treating the compound of Formula XIX with a compound of Formula II to give a compound of Formula XX; 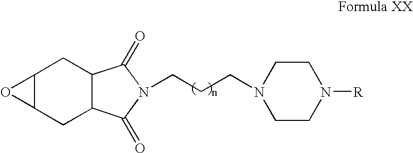
treating the compound of Formula XX with hydrochloric acid to give a compound of Formula XXI; and 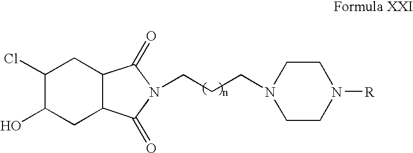
Treating the compound of Formula XXI with a fluorinating agent gives a compound of Formula XXII.

57. A process for the preparation of a compound of Formula XXIII, 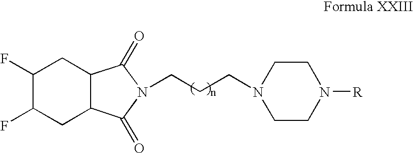
its pharmaceutically acceptable acid addition salts, solvates, enantiomers, diastereomers, regioisomers, N-oxides, polymorphs, prodrugs and metabolites wherein,
the variable n represents the integers 1 or 2; and
R is 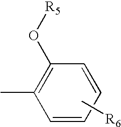
wherein R₅ is selected from alkyl or cycloalkyl, and wherein R₆ is selected from hydrogen, halogen or alkyl,
the method comprising:
reacting a compound of Formula XX with a fluorinating agent to give a compound of Formula XXIII, [chemical structure]
